(19) United States
(12) Patent Application Publication
(10) Pub. No.: US 2025/0066417 A9
(43) Pub. Date: Feb. 27, 2025

(54) NEW PEPTIDE-BASED DIAGNOSTIC AND THERAPEUTIC AGENTS

(71) Applicant: ENLITISA (SHANGHAI) PHARMACEUTICAL CO., LTD., Shanghai (CN)

(72) Inventors: Ming GU, Jiangyin, Jiangsu (CN); Maoqian SONG, Jiangyin, Jiangsu (CN); Bengt Ingemar SAMUELSSON, Solna (SE); Jan-Christer JANSON, Uppsala (SE)

(21) Appl. No.: 18/282,362

(22) PCT Filed: Mar. 17, 2022
(86) PCT No.: PCT/CN2022/081413

§ 371 Date: Sep. 15, 2023

(30) Foreign Application Priority Data
Mar. 17, 2021 (WO) PCT/CN2021/081362

(51) Int. Cl. C07K 7/06; A61K 38/00; A61K 51/08

(52) U.S. Cl. CPC C07K 7/06; A61K 51/088; A61K 38/00

(57) ABSTRACT

Provided is a modified peptide compound comprising one or more of the peptide components (a), (b) or (c): (a) a peptide component of formula I, A-Q-B I A and B represent Z or represent A1-1-B1, A1 and B1 independently represent Z or A2-Q2-B2, A2 and B2 independently represent Z or Z-Q3-Z, Q1, Q2 and Q3 represent, for example, Lys and Z represents a peptide component of the amino acid sequence: [W-Lys-X1-Ser-U-X2-Y] n-W-Lys-X1-Ser-U-X2-Y- (SEQ ID No: 3) (b) a peptide component of the amino acid sequence: [Ala-Lys-X1-Ser-U-X2-Y] p-Ala-Lys-X1-Ser-U-X2-Y-G (SEQ ID No: 4); or (c) a peptide component of the amino acid sequence: W1-Lys-X1-Ser-U1-X2-Y-G (SEQ ID No: 5) wherein: n p, W, W1, X1, U, U1, X2, Y and G have meanings given in the description, and wherein the peptide component is modified to include a labelling groups or labelling components that is/are capable of medical imaging of internal regions of the body non-invasively, and/or of diagnosing and/or therapeutically treating one or more diseases (such as cancers) in a patient.

### FIELD OF THE INVENTION

This Invention relates to new modified peptide compounds, the use of such compounds in human medicine, and to pharmaceutical compositions comprising them. In particular, the invention relates to the use of those modified peptide compounds in the treatment and/or diagnosis of e.g. cancers.

### BACKGROUND AND PRIOR ART

Medical imaging is a term for a set of techniques that are employed to provide visual representations of internal structures (body parts) and their physiology, enabling more accurate identification of abnormalities and therefore better diagnosis and treatment of diseases by medical intervention.

Medical imaging employs radiology to noninvasively produce Images of the internal aspects of the body. Imaging technologies that are employed Include magnetic resonance imaging, ultrasound, spectroscopy, X-ray radiography and functional imaging techniques that are employed in nuclear medicine.

The term ‘nuclear medicine’ refers to a medical technique that Involves the use of radioactive substances in the diagnosis and treatment of diseases.

Nuclear medicine imaging (also known as endoradiology) records radiation emitting from within the body from radiopharmaceuticals that are taken Intravenously or orally. External detectors (e.g. gamma cameras) capture Images from the radiation emitted.

Techniques employed Include scintigraphy, positron emission tomography (PET) and single-photon emission computed tomography (SPELT). The end result of nuclear medicine imaging comprises a ‘dataset’ of one or more Images, which can be represented as a time sequence.

Radionuclides can also be used therapeutically to treat conditions such as hyperthyroidism, and cancers, such a thyroid cancer, skin cancer and blood disorders. Again, radiopharmaceuticals are administered, either systemically (Intravenously or orally) or locally to the area to be treated. The local emission of ionizing radiation minimizes undesirable side effects and damage to nearby organs.

In the case of systemic delivery, it is Important that the delivery vehicle is tailored to selectively and rapidly associate with, for example, cancer cells and/or tumours to avoid such damage occurring, and there are numerous approaches that may be employed to do this, including employing peptides that selectively bind to receptors, such as neurotensin receptors. that are more common in cancer cells than elsewhere in the body. Commercial examples Include Lutathera®.

However, there remains a need for Improved medical imaging and nuclear medicine techniques.

Mussel adhesive protein (MAP), also known as Mytilus edulis foot protein (mefp), is a protein that is secreted by marine shellfish species, such as Mytilus edulis, Mytilus coruscus and Perna viridis. Eleven identified separate adhesive protein subtypes have been derived from mussels, including the collagens pre-COL-P, pre-COL-D and pre-COL-NG; the mussel feet matrix proteins PTMP (proximal thread matrix protein) and DTMP (distal thread matrix protein); and mfp proteins mfp-2 (sometimes referred to as “mefp-2”, hereinafter used Interchangeably), mfp-3/mefp-3, mfp-4/mefp-4, mfp-5/mefp-5, mfp-6/mefp-6 and, most preferably mfp-1/mefp-1 (see, for example, Zhu et a/., Advances in Marine Science, 2014, 32, 560-568 and Gao et al., Journal of Anhui Agr. Sci., 2011, 39, 19860-19862).

A significant portion of mefp-1 consists of 70 to 90 tandem repeats of the decapeptide: Ala-Lys-Pro-Ser-Tyr-Hyp-Hyp-Thr-DOPA-Lys (SEQ ID No: 1; see Waite, Int J. Adhesion and Adhesives, 1987, 7, 9-14). This decapeptide sequence may be Isolated as a low molecular weight derivative of naturally-occurring MAPS, or may be synthesized, for example as described by Yamamoto in J. Chem. Soc., Perkin Trans., 1987, 1, 613-618. See also Dalsin et a/., J. Am. Chem. Soc., 2003, 125, 4253-4258.

Analogues of the decapeptide, notably Ala-Lys-Pro-Ser-Tyr-Hyp-Hyp-Thr-Tyr-Lys (SEQ ID No: 2) have also been disclosed. See, for example, U.S. Pat. No. 5,616,311 and WO 96/39128.

To the applicant's knowledge, MAP and Its low molecular weight derivatives have never been employed in medical imaging or in nuclear medicine.

### DISCLOSURE OF THE INVENTION

According to a first aspect of the invention, there is provided a modified peptide compound comprising one or more of the peptide components (a), (b) or (c) as defined below:
(a) a peptide component of formula I,

A-Q-B I

wherein:
A and B Independently represent Z or A¹-Q¹-B¹;
Q represents a structural or formula II,

wherein:

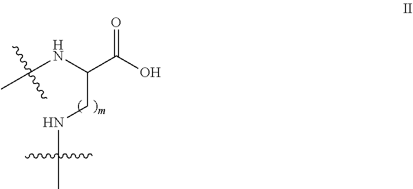

the squiggly lines represent points of attachment of Q to A and/or B; and
m represents an Integer 1 to 4;
A¹ and B¹ Independently represent Z or A²-Q²-B²;
A² and B² Independently represent Z or Z-Q³-Z;
Q¹, Q² and Q³ Independently represent structural fragments of formula III,

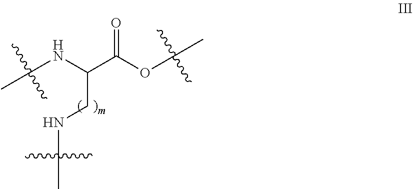

wherein:
the squiggly lines adjacent to the NH groups represent the points of attachment of Q¹, Q² and Q³ to A¹ and/or B¹, A² and/or B², and Z, respectively; and the squiggly line adjacent to the O atom represents the point of attachment of Q¹, Q² and Q³ to Q, Q¹ and Q², respectively; and m is as defined above;
on each occasion that it is employed, Z represents a peptide component of the amino acid sequence:

(SEQ ID NO: 3)
[W-Lys-X¹-Ser-U-X²-Y]ₙ-W-Lys-X¹-Ser-U-X²-Y---

wherein:
the dashed line represents the point of attachment of Z to the rest of the molecule;
n represents 0 or an Integer 1 to 4; and,
on each occasion that they are employed:
W represents a 1 or 2 amino acid sequence, in which the amino acids are selected from one or more of the group Lys, Ala, DOPA and a 3,4-dihydrocinnamic add (HCA) residue, provided that, when present, the HCA residue is located at the N-terminus of the peptide sequence Z;
X¹ represents Pro, Hyp or diHyp;
U represents Tyr or DOPA;
X² represents Ser, Pro, Hyp or diHyp; and
Y represents a 1 to 5 (e.g. a 1 to 4) amino acid sequence, in which the amino acids are selected from one or more of the group Lys, Ala, Pro, Hyp, diHyp, Thr, DOPA and Tyr; or
(b) a peptide component of the amino acid sequence:

(SEQ ID NO: 4)
[Ala-Lys-X¹-Ser-U-X²-Y]_{P}-Ala-Lys-X¹-Ser-U-X²-Y-G

wherein
p represents an Integer 1 to 4;
G may be absent (in which case Y is the C-terminal amino acid) or G may represent DOPA or dopamine (or, more properly, ‘a dopamine fragment’); and
X¹, U, X² and Y are as defined above; or
(c) a peptide component of the amino acid sequence:

(SEQ ID NO: 5)
W¹-Lys-X¹-Ser-U¹-X²-Y-G

wherein:
W¹ is absent (in which case Lys is the N-terminal amino acid), or represents a 1, 2 or 3 amino acid sequence, in which the amino adds are selected from one or more of the group Ser, Lys, Ala, DOPA and a 3,4-dihydrocinnamic acid (HCA) residue, provided that, when present, the HCA residue is located at the N-terminus of the peptide sequence;
U¹ represents Tyr, DOPA or a single bond (i.e. is absent); and
X¹, X², Y and G are as defined above,
wherein said one or more peptide components are modified to Include a labelling group or a labelling component, which labelling group or component is capable of (e.g. non-invasive) medical imaging of an (e.g. Internal) area of a human or animal body of a patient, and/or human or animal body parts, and/or is capable of diagnosing and/or therapeutically treating one or more disease, such as a cancer, in a patient,
as well as regioisomers, stereoisomers, and pharmaceutically-acceptable salts of said compounds, which compounds, regioisomers, stereoisomers and salts are referred to together hereinafter as ‘the compounds of the invention’.

The peptide components (a), (b) and/or (c) may be modified to Include a labelling group/component (which may also be termed as an ‘effector’ group or component) that may assist in the non-invasive imaging of parts of a human or animal body and/or imaging of biological processes in a patient with the assistance of known medical imaging and/or nuclear medicine techniques, such as those described herein.

Labelling groups/components of compounds of the invention may thus comprise a molecule that is capable of medically imaging parts of the body including vasculature networks in Internal organs, including cancer cells and/or tumours, by virtue of one of more physical properties that it possesses, such as the ability to exhibit bioluminescence.

Such molecules may Include things like UV-exdtable fluorophores, fluoresceins, rhodamines, naphthoxanthene dyes, phenanthridines, BODIPY dyes, cyanines, phthalocyanines, naphthalocyanines, xanthenes, acridines, oxazines, polyenes, oxonols, benzimidazoles, azamethines, styryls, thiazoles, anthraquinones, naphthalimides, aza[18]annulenes, porphins, squaraines, 8-hydroxyquinoline derivatives, polymethins, perylenes, upconversion dyes, diketopyrrolopyrroles, porphyrins (such as hematoporphyrin derivatives, benzoporphyrin derivatives), 5-aminolevulinic acid and texaphyrins, a molecule of the chlorophyll family (such as chlorin), purpurins, bacteriochlorins and metalloporphyrins.

Preferred labelling components of this type Include fluorescein and derivatives thereof, such as carboxyfluorescein (e.g. 5- or 6-carboxyfluorescein), carboxyfluorescein diacetate succinimidyl ester, carboxyfluorescein succinimidyl ester, naphthofluorescein, carboxynaphthofluorescein (e.g. 5- or 6-carboxynaphthofluorescein), 2,7-dichlorofluorescein, carboxy-2,7-dichlorofluorescein (e.g. 5- or 6-carboxy-2,7-dichlorofluoresceine and 5- or 6-carboxy-2′,7′-dichlorofluorescein), 5-chloromethylfluorescein, fluorescein isothiocyanate (e.g. 5- or 6-fluorescein isothiocyanate), fluorescein-5-EX and fluorescein-dibase.

When such labelling components of this type are coupled to the peptide component as defined above, a linker moiety may be employed, such as an amino acid (e.g. a linear amino acid) such as an aminocaprioic add, e.g. 6-aminocaprioic acid (hereinafier‘ACP’).

Such labelling components may thus be used in medical imaging in numerous ways, which are well known to those skilled in the art, and/or may therefore provide compounds of the invention with the ability to diagnose the existence, size and nature of medical conditions, including cancers and/or tumours. Furthermore, some of the above substances may also be photosensitizers or sensitizers or amplifiers of other combined conventional cancer treatment methods and may therefore provide compounds of the Invention with the ability to therapeutically treat cancers/tumours.

The labelling group or component of a compound of the invention may in the alternative comprise one or more tracer components that are capable of radionuclide imaging. For example, one of more of the essential, ¹H, ¹²C, ¹⁴N and/or ¹⁶O atoms in a peptide components may be replaced by one or more Isotopes of those atoms, such as ³H, ¹¹C, ¹³C, ¹⁴C., ¹³N and¹⁵O, for example as described hereinafter, which Isotopes which may thus comprise the labelling group or component of a compound of the invention.

Alternatively, or in addition, one or more of the free functional groups in a peptide component may be reacted with, or may be conjugated to, other radioactive Isotope (radionuclide) atoms that are known to be useful in the diagnosis, imaging and/or treatment of a diseases, such as cancer, by known techniques.

Radioactive Isotope atoms that may be employed in the context of the present invention may thus be selected from the group: ¹⁸F, ²²Na, ²⁴Na, ³²P, ³³p, ⁴²K, ⁴⁷Ca, ⁴⁷Sc, ⁵¹Cr, ⁵⁷Co, ⁵⁸Co, ⁵⁹Fe, ⁶⁰Co, ⁶⁴CU, ⁶⁷CU, ⁶⁷Ga, ⁶⁸Ga, ⁷⁵Se, ⁷⁷As, ^{80m}Br, ^{81m}Kr, ⁸²Rb, ⁸⁹Sr, ⁸⁹Zr, ⁹⁰Y, ⁹⁰Sr, ⁹⁹Mo, ^{99m}TC, ¹⁰³Pd, ^{103m}Rh, ¹⁰⁵Rh, ¹⁰⁹Pd, ¹⁰⁹Pt, ¹¹¹Ag, ¹¹¹In, ¹¹⁹Sb, ¹²¹Sn, ¹²⁷Te, ¹²³I, ¹²⁵I, ¹²⁹I, ¹³¹I, ¹³³Xe, ¹⁴²Pr, ¹⁴³Pr, ¹⁴⁹Pm, ¹⁵¹Pm, ¹⁵²Dy, ¹⁵³Sm, ¹⁵⁹Gd, ¹⁶¹Tb, ¹⁶¹Ho, ¹⁶⁵Dy, ¹⁶⁶Ho, ¹⁶⁶Dy, ¹⁶⁹Er, ¹⁶⁹Yb, ¹⁷⁵Yb, ¹⁷²Tm, ¹⁷⁷Lu, ^{177m}Sn, ¹⁸⁶Re, ¹⁸⁸Re, ¹⁸⁹Re, ¹⁸⁸Rd, ^{189m}Os, ¹⁹²Ir, ¹⁹⁴Ir, ¹⁹⁸Au, ¹⁹⁹Au, ²⁰¹TI, ²¹¹At, ²¹¹Pb, ²¹²Pb, ²¹¹Bi, ²¹²Bi, ²¹³Bi, ²¹⁵Po, ²¹⁷At, ²¹⁹Rn, ²²¹Fr, ²²³Ra, ²²⁵Ac, ²²⁷Th and ²⁵⁵Fm.

Other radioactive Isotopes that may be employed in the context of the present invention Include ¹²⁴I.

As described hereinafter, such radionuclides may be bound, bonded or reacted directly to peptide components of compounds of the invention, and may thus comprise the labelling component in their own right.

Conversely, peptide components may be reacted with, or may be conjugated to, another molecule that is capable of reacting with, or forming a complexing with, such a radioactive Isotope. Radionuclides may in this way be presented in the form of a complex, which complex comprises the labelling component of the compound of the invention. Molecules that are capable of forming complexes with (particularly heavy metallic) radionuclides are well known in the art and Include things like diethylene triamine pentaacetic acid (DTPA) or 1, 4, 7, 10-tetraazacyclododecane-1, 4, 7, 10-tetraacetic acid (DOTA), 1,4,7-triazacyciononanetriacetic add (NOTA), ethylenedlamine-N,N′-tetraacetic acid (EDTA), 1,4,8,11-tetraazacydododecane-1,4,8,11-tetraacetic acid (TETA), 1,4,7,10 tetraazacyclotridecane-1,4,7,10-tetraacetic acid (TRITA), trans- 1,2-diaminocyclohexane-N,N,N′,N′-tetraacetic acid (CDTA), 6-hydrazinonicotinic acid (HYNIC), 1,4,7-triazacyclononane-N-glutaric acid-N′,N′-diacetic add (NODAGA), 1,4,7-triazacyclononane-1-succinic acid-4,7-diacetic acid (NODASA), bisaminobisthiols and desferal or desferrioxamine-type chelators, all of which may be coupled to peptide components of compounds of the invention by known techniques, e.g. using well-known linker groups such as those described herein.

Further molecules that are capable of forming complexes with (particularly heavy metallic) radionuclides Include diacetyl-bis(N(4)-methylthiosemicarbazonate (ATSM), pyruvaldehyde bis(N(4)-methylthiosemicarbazone (PTSM) and 2,2′-(1,4,8,11-tetraazabicyclo[6.6. 2]hexadecane-4,11-diyl)diacetic acid (CB-TE2A).

Compounds of the invention may comprise one or more labelling groups or labelling components as appropriate, which may be the same or may be different in their structure and/or purpose.

Peptide components (c) of compounds of the invention that may be mentioned Include those in which:
W¹ represents W as hereinbefore defined;
U¹ represents U as hereinbefore defined;
Y represents a 1 to 5 (e.g. a 1 to 4) amino acid sequence, in which the amino acids are selected from one or more of the group Lys, Ala, Pro, Hyp, Thr, DOPA and Tyr.

Preferred compounds of the invention include those in which:
X¹ represents Hyp or, more preferably, Pro;
X² represents Ser, Pro or, more preferably, Hyp;
W and/or W¹ represents HCA, HCA-Ala-, preferably Ala or Lys-Ala or, more preferably DOPA or DOPA-Ala-; and/or
Y represents a 5, preferably a 3 or, more preferably, a 4 amino acid sequence, in which the amino acids are selected from one or more of the group Lys, Ala, Hyp, Thr, DOPA and Tyr.

More preferably, compounds of the invention include those in which Y represents a 4 amino acid sequence selected from the group -Pro-V¹-Y²-Lys- or, more preferably, -Hyp-Y¹-Y²-Lys- and -Thr-Y¹-Y²-Lys-, wherein Y¹ and Y² are each independently selected from the group Pro or, more preferably, Ala, Hyp, Thr, DOPA and Tyr.

Wherein Y represents a 4 amino acid sequence, preferred compounds of the invention include those in which the amino acid sequence defined by Y is selected from the group:
-Pro-Thr-DOPA-Lys-;
-Pro-Thr-Tyr-Lys-;
-Thr-Tyr-Pro-Lys-;
-Thr-DOPA-Pro-Lys-; and, more preferably,
-Hyp-Thr-Tyr-Lys-;
-Hyp-Thr-DOPA-Lys-;
-Hyp-Thr-Ala-Lys-;
-Thr-Tyr-Hyp-Lys-;
-Thr-DOPA-Hyp-Lys-; and
-Thr-Ala-Hyp-Lys-.

Wherein Y represents a 4 amino add sequence, other preferred compounds of the invention include those in which the amino acid sequence defined by Y is selected from the group:
-Thr-Tyr-DOPA-Lys-.

When Y represents a 2 amino acid sequence, preferred compounds of the invention Include those in which the amino acid sequence defined by Y is selected from the group -Hyp-Thr-, -Thr-Tyr-, -Pro-Thr- and -Thr-DOPA-.

Other preferred compounds of the invention that may be mentioned include those in which the amino acid sequence defined by Y is selected from -Thr-Tyr-Lys-, -Tyr-Pro-Lys-, -DOPA-Pro-Lys-, -Hyp-Thr-Tyr-, -Hyp-Thr-Tyr-Hyp-Lys- and, more preferably, the groups -Thr-Tyr-Hyp-Lys-DOPA- and -Hyp-Thr-DOPA-.

When compounds of the invention comprise peptide components of formula I (as defined under (a) above), those that may be mentioned are those wherein m represents 1, 3 or, more preferably 4, such that one or more of Q, Q¹, Q² and Q³ represent Lys or, more properly, ‘a Lys fragment’, in accordance with what are defined above as ‘the structural fragments of formulae II and III’ (as appropriate).

On each occasion that they are employed, Q, Q¹, Q² and Q³ may each be attached to zero, one or two Z groups.

In this respect, preferred compounds of the invention Include those in which, in the peptide component of formula I:
one of A or B represents Z and the other represents A¹-Q¹-B¹; or, more preferably,
A and B both represent Z, or both represent A¹-Q¹-B¹,
in which, in each case, Q¹ preferably represents a Lys fragment and Z is as hereinbefore defined.

Further preferred compounds of the invention also Include those in which:
one of A¹ and B¹ represents Z and the other represents A²-Q²-B²; or, more preferably,
A¹ and B¹ both represent Z, or both represent A²-Q²-B²,
in which, in each case, Q² preferably represents a Lys fragment, and Z is as hereinbefore defined.

Further preferred compounds of the invention also Include those in which: one of A² and B² represents Z and the other represents Z-Q³-Z; or, more preferably, A² and B² both represent Z, or both represent Z-Q³-Z, in which, in each case, Q³ preferably represents a Lys fragment, and Z is as hereinbefore defined.

More preferred compounds of the invention Include those in which A² and B² both represent Z.

Peptide components of compounds of the invention that may be mentioned Include those in which n is 0, 1 or 4, or, more preferably, n is 0.

When compounds of the invention comprises peptide components as defined under (b) or (c) above, the terms ‘dopamine’ and ‘dopamine fragment’ that may be defined by the substituent G refer to a structural fragment of formula IV,

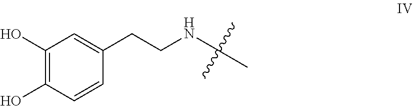 [SMILES: *NCCc1ccc(O)c(O)c1]

wherein the squiggly line represents the point of attachment to Y. PGP-26,CI

Preferred values of p in peptide components as defined under (b) above are, in ascending order of preference 2, 3, 1 and 4.

Particular compounds of the invention comprising peptide components as defined under (b) above that may be mentioned are those where G is absent and, in this respect, preferred peptide components include those of the amino acid sequence:

(SEQ ID No: 6)
Ala-Lys-Pro-Ser-Tyr-Ser-Hyp-Thr-Tyr-Lys-
Ala-Lys-Pro-Ser-Tyr-Ser-Hyp-Thr-Tyr-Lys;
(SEQ ID No: 7)
Ala-Lys-Pro-Ser-Tyr-Hyp-Hyp-Thr-Tyr-Lys-
Ala-Lys-Pro-Ser-Tyr-Hyp-Hyp-Thr-Tyr-Lys-
Ala-Lys-Pro-Ser-Tyr-Hyp-Hyp-Thr-Tyr-Lys-
Ala-Lys-Pro-Ser-Tyr-Hyp-Hyp-Thr-Tyr-Lys-
Ala-Lys-Pro-Ser-Tyr-Hyp-Hyp-Thr-Tyr-Lys;
(SEQ ID No: 8)
Ala-Lys-Pro-Ser-DOPA-Hyp-Hyp-Thr-DOPA-
Lys-Ala-Lys-Pro-Ser-DOPA-Hyp-Hyp-Thr-
DOPA-Lys;
(SEQ ID No: 9)
Ala-Lys-Pro-Ser-DOPA-Hyp-Hyp-Thr-DOPA-
Lys-Ala-Lys-Pro-Ser-DOPA-Hyp-Hyp-Thr-
DOPA-Lys-Ala-Lys-Pro-Ser-DOPA-Hyp-Hyp-
Thr-DOPA-Lys-Ala-Lys-Pro-Ser-DOPA-Hyp-
Hyp-Thr-DOPA-Lys-Ala-Lys-Pro-Ser-DOPA-
Hyp-Hyp-Thr-DOPA-Lys;
(SEQ ID No: 10)
Ala-Lys-Pro-Ser-Tyr-Hyp-Hyp-Thr-DOPA-
Lys-Ala-Lys-Pro-Ser-Tyr-Hyp-Hyp-Thr-
DOPA-Lys;
(SEQ ID No: 11)
Ala-Lys-Pro-Ser-Tyr-Hyp-Hyp-Thr-DOPA-
Lys-Ala-Lys-Pro-Ser-Tyr-Hyp-Hyp-Thr-
DOPA-Lys-Ala-Lys-Pro-Ser-Tyr-Hyp-Hyp-
Thr-DOPA-Lys;
(SEQ ID No: 12)
Ala-Lys-Pro-Ser-Tyr-Hyp-Hyp-Thr-DOPA-
Lys-Ala-Lys-Pro-Ser-Tyr-Hyp-Hyp-Thr-
DOPA-Lys-Ala-Lys-Pro-Ser-Tyr-Hyp-Hyp-
Thr-DOPA-Lys-Ala-Lys-Pro-Ser-Tyr-Hyp-
Hyp-Thr-DOPA-Lys;
and
(SEQ ID No: 13)
Ala-Lys-Pro-Ser-Tyr-Hyp-Hyp-Thr-DOPA-
Lys-Ala-Lys-Pro-Ser-Tyr-Hyp-Hyp-Thr-
DOPA-Lys-Ala-Lys-Pro-Ser-Tyr-Hyp-Hyp-
Thr-DOPA-Lys-Ala-Lys-Pro-Ser-Tyr-Hyp-
Hyp-Thr-DOPA-Lys-Ala-Lys-Pro-Ser-Tyr-
Hyp-Hyp-Thr-DOPA-Lys.

Compounds of the invention that may be mentioned Include those in which:
X¹ represents Pro;
U and/or U¹ represents Tyr; and/or
W and/or W¹ represents Ala, and, in this respect, compounds of the invention comprising:
peptide components of formula I as defined under (a) above that may be mentioned Include those wherein Z is selected from the group:

(SEQ ID No: 2)
Ala-Lys-Pro-Ser-Tyr-Hyp-Hyp-Thr-Tyr-Lys---;
(SEQ ID No: 1)
Ala-Lys-Pro-Ser-Tyr-Hyp-Hyp-Thr-DOPA-Lys---;
(SEQ ID No: 14)
Ala-Lys-Pro-Ser-Tyr-Hyp-Thr-Tyr-Hyp-Lys---;
(SEQ ID No: 15)
Ala-Lys-Pro-Ser-Tyr-Hyp-Thr-DOPA-Hyp-Lys---;
(SEQ ID No: 16)
Ala-Lys-Pro-Ser-Tyr-Hyp-Thr-Tyr-Hyp-Lys-DOPA---;
(SEQ ID No: 17)
Ala-Lys-Pro-Ser-Tyr-Ser-Hyp-Thr-Tyr-Lys-Ala-Lys-
Pro-Ser-Tyr-Ser-Hyp-Thr-Tyr-Lys--;
and
(SEQ ID No: 18)
Ala-Lys-Pro-Ser-Tyr-Hyp-Hyp-Thr-Tyr-Lys-Ala-
Lys-Pro-Ser-Tyr-Hyp-Hyp-Thr-Tyr-Lys-Ala-Lys-
Pro-Ser-Tyr-Hyp-Hyp-Thr-Tyr-Lys-Ala-Lys-Pro-
Ser-Tyr-Hyp-Hyp-Thr-Tyr-Lys-Ala-Lys-Pro-Ser-
Tyr-Hyp-Hyp-Thr-Tyr-Lys---;

and peptide components as defined under (c) above that may be mentioned Include those of

(SEQ ID No: 19)
Ala-Lys-Pro-Ser-Tyr-Hyp-Thr-Tyr-Hyp-Lys-DOPA;
(SEQ ID No: 20)
Ala-Lys-Pro-Ser-Tyr-Hyp-Thr-Tyr-Hyp-Lys-Dopamine;
(SEQ ID No: 21)
Ala-Lys-Pro-Ser-Tyr-Hyp-Hyp-Thr-Tyr-Lys-Dopamine;
(SEQ ID No: 22)
Ala-Lys-Pro-Ser-Tyr-Pro-Pro-Thr-DOPA-Lys;
(SEQ ID No: 23)
Ala-Lys-Pro-Ser-Tyr-Pro-Thr-Tyr-Pro-Lys;
(SEQ ID No: 24)
Ala-Lys-Pro-Ser-Tyr-Pro-Thr-DOPA-Pro-Lys;
(SEQ ID No: 25)
Ala-Lys-Pro-Ser-Tyr-Pro-Hyp-Thr-Tyr-Lys;
(SEQ ID No: 26)
Ala-Lys-Pro-Ser-Tyr-Pro-Hyp-Thr-DOPA-Lys;
(SEQ ID No: 27)
Ala-Lys-Pro-Ser-Tyr-Hyp-Pro-Thr-Tyr-Lys;
(SEQ ID No: 28)
Ala-Lys-Pro-Ser-Tyr-Hyp-Pro-Thr-DOPA-Lys;
(SEQ ID No: 29)
Ala-Lys-Pro-Ser-Tyr-Hyp-Thr-Tyr-Hyp-Lys;
(SEQ ID No: 30)
Ala-Lys-Pro-Ser-Tyr-Hyp-Thr-DOPA-Hyp-Lys;
(SEQ ID No: 31)
Ala-Lys-Pro-Ser-Tyr-Hyp-Hyp-Thr-Tyr-Lys-DOPA;
(SEQ ID No: 32)
Ala-Lys-Pro-Ser-Tyr-Hyp-Hyp-Thr-DOPA-Lys-DOPA;
(SEQ ID No: 33)
Ala-Lys-Pro-Ser-Tyr-Hyp-Hyp-Thr-DOPA-Lys-Dopamine;
(SEQ ID No: 34)
Ala-Lys-Pro-Ser-Tyr-Hyp-Thr-DOPA-Hyp-Lys-DOPA;
and
(SEQ ID No: 35)
Ala-Lys-Pro-Ser-Tyr-Hyp-Thr-DOPA-Hyp-Lys-Dopamine.

Compounds of the invention that may be mentioned Include those in which:
U and/or U¹ represents Tyr;
X² represents Hyp; and/or
W and/or W¹ represents Lys-Ala-, and, in this respect, compounds of the invention comprising:
peptide components of formula I as defined under (a) above that may be mentioned Include those wherein Z is selected from the group:

(SEQ ID No: 36)
Lys-Ala-Lys-Pro-Ser-Tyr-Hyp-Hyp-Thr-Tyr---;
(SEQ ID No: 37)
Lys-Ala-Lys-Hyp-Ser-Tyr-Hyp-Hyp-Thr-DOPA---;
and
(SEQ ID No: 38)
Lys-Ala-Lys-Pro-Ser-Tyr-Hyp-Hyp-Thr-DOPA---;

and
peptide components as defined under (c) above that may be mentioned Include those of the amino acid sequence:

(SEQ ID No: 39)
Lys-Ala-Lys-Pro-Ser-Tyr-Hyp-Hyp-Thr-DOPA;
(SEQ ID No: 40)
Lys-Ala-Lys-Pro-Ser-Tyr-Hyp-Hyp-Thr-Dopamine;
(SEQ ID No: 41)
Lys-Ala-Lys-Hyp-Ser-Tyr-Hyp-Hyp-Thr-DOPA;
(SEQ ID No: 42)
Lys-Ala-Lys-Hyp-Ser-Tyr-Hyp-Hyp-Thr-Tyr;
and
(SEQ ID NO: 43)
Lys-Ala-Lys-Pro-Ser-Tyr-Hyp-Hyp-Thr-Tyr.

Further compounds of the invention that may be mentioned Include those in which:
X¹ represents Pro;
U and/or U¹ represents Tyr;
X² represents Hyp; and/or
W and/or W¹ represents HCA, HCA-Ala- or, more preferably, DOPA or DOPA-Ala-, and, in this respect, compounds of the invention comprising:
peptide components of formula I as defined under (a) above that may be mentioned Include those wherein Z is selected from the group:

(SEQ ID No: 44)
HCA-Ala-Lys-Pro-Ser-Tyr-Hyp-Thr-Tyr-Hyp-Lys---;
(SEQ ID No: 45)
HCA-Ala-Lys-Pro-Ser-Tyr-Hyp-Hyp-Thr-Tyr-Lys ---;
(SEQ ID No: 46)
HCA-Lys-Pro-Ser-Tyr-Hyp-Hyp-Thr-Ala-Lys---;
(SEQ ID No: 47)
HCA-Lys-Pro-Ser-Tyr-Hyp-Thr-Ala-Hyp-Lys---;
(SEQ ID No: 48)
DOPA-Lys-Pro-Ser-Tyr-Hyp-Thr-Ala-Hyp-Lys---;
(SEQ ID No: 49)
DOPA-Lys-Pro-Ser-Tyr-Hyp-Hyp-Thr-Ala-Lys---;
(SEQ ID No: 50)
DOPA-Ala-Lys-Pro-Ser-Tyr-Hyp-Thr-Tyr-Hyp-Lys---;
and
(SEQ ID No: 51)
DOPA-Ala-Lys-Pro-Ser-Tyr-Hyp-Hyp-Thr-Tyr-Lys---;

and
peptide components as defined under (c) above that may be mentioned Include those of the amino acid sequence:

(SEQ ID No: 52)
DOPA-Lys-Pro-Ser-Tyr-Hyp-Thr-Ala-Hyp-Lys;
(SEQ ID No: 53)
DOPA-Lys-Pro-Ser-Tyr-Hyp-Hyp-Thr-Ala-Lys;
(SEQ ID No: 54)
DOPA-Ala-Lys-Pro-Ser-Tyr-Hyp-Thr-Tyr-Hyp-Lys;
(SEQ ID No: 55)
DOPA-Ala-Lys-Pro-Ser-Tyr-Hyp-Hyp-Thr-Tyr-Lys;
(SEQ ID No: 56)
DOPA-Ala-Lys-Pro-Ser-Tyr-Hyp-Thr-DOPA-Hyp-Lys;
(SEQ ID No: 57)
DOPA-Ala-Lys-Pro-Ser-Tyr-Hyp-Hyp-Thr-DOPA-Lys;
(SEQ ID No: 58)
HCA-Lys-Pro-Ser-Tyr-Hyp-Hyp-Thr-Ala-Lys;
(SEQ ID No: 59)
HCA-Ala-Lys-Pro-Ser-Tyr-Hyp-Thr-Tyr-Hyp-Lys;
(SEQ ID No: 60)
HCA-Ala-Lys-Pro-Ser-Tyr-Hyp-Thr-DOPA-Hyp-Lys;
(SEQ ID No: 61)
HCA-Ala-Lys-Pro-Ser-Tyr-Hyp-Hyp-Thr-Tyr-Lys;
(SEQ ID No: 62)
HCA-Ala-Lys-Pro-Ser-Tyr-Hyp-Hyp-Thr-DOPA-Lys;
and
(SEQ ID NO: 63)
HCA-Lys-Pro-Ser-Tyr-Hyp-Thr-Ala-Hyp-Lys.

Other compounds of the invention that may be mentioned Include those in which:
U and/or U¹ represents DOPA; and/or
W and/or W¹ represents Ala or Lys-Ala-, and, in this respect, compounds of the invention comprising peptide components of formula I as defined under (a) above that may be mentioned include those wherein Z is selected from the group:

(SEQ ID No: 64)
Ala-Lys-Pro-Ser-DOPA-Hyp-Thr-DOPA-Hyp-Lys---;
(SEQ ID No: 65)
Ala-Lys-Pro-Ser-DOPA-Hyp-Hyp-Thr-DOPA-Lys---;
(SEQ ID No: 66)
Lys-Ala-Lys-Pro-Ser-DOPA-Hyp-Hyp-Thr-DOPA---;
(SEQ ID No: 67)
Lys-Ala-Lys-Hyp-Ser-DOPA-Hyp-Hyp-Thr-DOPA---;
(SEQ ID No: 68)
Ala-Lys-Pro-Ser-DOPA-Hyp-Hyp-Thr-DOPA-Lys-
Ala-lys-Pro-Ser-DOPA-Hyp-Hyp-Thr-DOPA-Lys---;
and
(SEQ ID No: 69)
Ala-Lys-Pro-Ser-DOPA-Hyp-Hyp-Thr-DOPA-
Lys-Ala-Lys-Pro-Ser-DOPA-Hyp-Hyp-Thr-
DOPA-Lys-Ala-Lys-Pro-Ser-DOPA-Hyp-Hyp-
Thr-DOPA-Lys-Ala-Lys-Pro-Ser-DOPA-Hyp-
Hyp-Thr-DOPA-Lys-Ala-Lys-Pro-Ser-DOPA-
Hyp-Hyp-Thr-DOPA-Lys---;

and
peptide components as defined under (c) above that may be mentioned Include those of the amino acid sequence:

(SEQ ID No: 70)
Lys-Ala-Lys-Pro-Ser-DOPA-Hyp-Hyp-Thr-Dopamine;
(SEQ ID No: 71)
Lys-Ala-Lys-Hyp-Ser-DOPA-Hyp-Hyp-Thr-DOPA;
(SEQ ID No: 72)
Lys-Ala-Lys-Pro-Ser-DOPA-Hyp-Hyp-Thr-DOPA;
(SEQ ID No: 73)
Ala-Lys-Pro-Ser-DOPA-Pro-Pro-Thr-Tyr-Lys;
(SEQ ID No: 74)
Ala-Lys-Pro-Ser-DOPA-Pro-Pro-Thr-DOPA-Lys;
(SEQ ID No: 75)
Ala-Lys-Pro-Ser-DOPA-Pro-Thr-Tyr-Pro-Lys;
(SEQ ID No: 76)
Ala-Lys-Pro-Ser-DOPA-Pro-Thr-DOPA-Pro-Lys;
(SEQ ID No: 77)
Ala-Lys-Pro-Ser-DOPA-Pro-Hyp-Thr-Tyr-Lys;
(SEQ ID No: 78)
Ala-Lys-Pro-Ser-DOPA-Pro-Hyp-Thr-DOPA-Lys;
(SEQ ID No: 79)
Ala-Lys-Pro-Ser-DOPA-Hyp-Pro-Thr-Tyr-Lys;
(SEQ ID No: 80)
Ala-Lys-Pro-Ser-DOPA-Hyp-Pro-Thr-DOPA-Lys;
(SEQ ID No: 81)
Ala-Lys-Pro-Ser-DOPA-Hyp-Thr-Tyr-Hyp-Lys;
(SEQ ID No: 82)
Ala-Lys-Pro-Ser-DOPA-Hyp-Thr-DOPA-Hyp-Lys;
(SEQ ID No: 83)
Lys-Ala-Lys-Pro-Ser-DOPA-Hyp-Hyp-Thr-Tyr;
(SEQ ID No: 84)
Lys-Ala-Lys-Hyp-Ser-DOPA-Hyp-Hyp-Thr-Tyr;
(SEQ ID No: 85)
Ala-Lys-Pro-Ser-DOPA-Hyp-Hyp-Thr-Tyr-Lys-DOPA;
(SEQ ID No: 86)
Ala-Lys-Pro-Ser-DOPA-Hyp-Hyp-Thr-DOPA-Lys-DOPA;
(SEQ ID No: 87)
Ala-Lys-Pro-Ser-DOPA-Hyp-Hyp-Thr-Tyr-Lys-Dopamine;
(SEQ ID No: 88)
Ala-Lys-Pro-Ser-DOPA-Hyp-Hyp-Thr-DOPA-Lys-Dopamine;
(SEQ ID No: 89)
Ala-Lys-Pro-Ser-DOPA-Hyp-Thr-Tyr-Hyp-Lys-DOPA;
(SEQ ID No: 90)
Ala-Lys-Pro-Ser-DOPA-Hyp-Thr-DOPA-Hyp-Lys-DOPA;
(SEQ ID No: 91)
Ala-Lys-Pro-Ser-DOPA-Hyp-Thr-Tyr-Hyp-Lys-Dopamine;
and
(SEQ ID NO: 92)
Ala-Lys-Pro-Ser-DOPA-Hyp-Thr-DOPA-Hyp-Lys-Dopamine.

Further compounds of the invention that may be mentioned Include those in which:
X¹ represents Pro;
U and/or U¹ represents DOPA;
X² represents Hyp; and/or
W and/or W¹ represents HCA, HCA-Ala- or, more preferably, DOPA or DOPA-Ala-, and, in this respect, particular compounds of the Invention comprising peptide components of formula I as defined under (a) above that may be mentioned Include those wherein Z is selected from the group:

(SEQ ID No: 93)
HCA-Ala-Lys-Pro-Ser-DOPA-Hyp-Thr-DOPA-Hyp-Lys---;
(SEQ ID No: 94)
HCA-Ala-Lys-Pro-Ser-DOPA-Hyp-Hyp-Thr-DOPA-Lys---;
(SEQ ID No: 95)
HCA-Lys-Pro-Ser-DOPA-Hyp-Thr-Ala-Hyp-Lys---;
(SEQ ID No: 96)
HCA-Lys-Pro-Ser-DOPA-Hyp-Hyp-Thr-Ala-Lys---;
(SEQ ID No: 97)
DOPA-Lys-Pro-Ser-DOPA-Hyp-Thr-Ala-Hyp-Lys---;
(SEQ ID No: 98)
DOPA-Lys-Pro-Ser-DOPA-Hyp-Hyp-Thr-Ala-Lys---;
(SEQ ID No: 99)
DOPA-Ala-Lys-Pro-Ser-DOPA-Hyp-Thr-DOPA-Hyp-Lys---;
(SEQ ID No: 100)
DOPA-Ala-Lys-Pro-Ser-DOPA-Hyp-Hyp-Thr-Tyr-Lys---;
and
(SEQ ID No: 101)
DOPA-Ala-Lys-Pro-Ser-DOPA-Hyp-Hyp-Thr-DOPA-Lys---;

peptide components as defined under (c) above that may be mentioned Include those of the amino acid sequence:

(SEQ ID No: 102)
DOPA-Lys-Pro-Ser-DOPA-Hyp-Thr-Ala-Hyp-Lys;
(SEQ ID No: 103)
DOPA-Lys-Pro-Ser-DOPA-Hyp-Hyp-Thr-Ala-Lys;
(SEQ ID No: 104)
DOPA-Ala-Lys-Pro-Ser-DOPA-Hyp-Thr-DOPA-Hyp-Lys;
(SEQ ID No: 105)
DOPA-Ala-Lys-Pro-Ser-DOPA-Hyp-Hyp-Thr-Tyr-Lys;
(SEQ ID No: 106)
DOPA-Ala-Lys-Pro-Ser-DOPA-Hyp-Hyp-Thr-DOPA-Lys.
(SEQ ID No: 107)
HCA-Ala-Lys-Pro-Ser-DOPA-Hyp-Thr-DOPA-Hyp-Lys;
(SEQ ID No: 108)
HCA-Ala-Lys-Pro-Ser-DOPA-Hyp-Hyp-Thr-DOPA-Lys;
(SEQ ID No: 109)
HCA-Lys-Pro-Ser-DOPA-Hyp-Thr-Ala-Hyp-Lys;
(SEQ ID No: 110)
HCA-Lys-Pro-Ser-DOPA-Hyp-Hyp-Thr-Ala-Lys;
(SEQ ID No: 111)
DOPA-Ala-Lys-Pro-Ser-DOPA-Hyp-Thr-Tyr-Hyp-Lys;
(SEQ ID No: 112)
HCA-Ala-Lys-Pro-Ser-DOPA-Hyp-Thr-Tyr-Hyp-Lys; and
(SEQ ID NO: 113)
HCA-Ala-Lys-Pro-Ser-DOPA-Hyp-Hyp-Thr-Tyr-Lys.

When compounds of the invention comprise a peptide component of formula I as defined under (a) above, those that may be mentioned Include those in which, in the peptide component of formula I:
A and B both represent Z;
one, or preferably both, Z groups represent:

(SEQ ID No: 44)
HCA-Ala-Lys-Pro-Ser-Tyr-Hyp-Thr-Tyr-Hyp-Lys---,
(SEQ ID No: 47)
HCA-Lys-Pro-Ser-Tyr-Hyp-Thr-Ala-Hyp-Lys---,
(SEQ ID No: 50)
DOPA-Ala-Lys-Pro-Ser-Tyr-Hyp-Thr-Tyr-Hyp-Lys---,
(SEQ ID No: 64)
Ala-Lys-Pro-Ser-DOPA-Hyp-Thr-DOPA-Hyp-Lys---,
(SEQ ID No: 66)
Lys-Ala-Lys-Pro-Ser-DOPA-Hyp-Hyp-Thr-DOPA---
(SEQ ID No: 93)
HCA-Ala-Lys-Pro-Ser-DOPA-Hyp-Thr-DOPA-Hyp-Lys---,
(SEQ ID No: 98)
DOPA-Lys-Pro-Ser-DOPA-Hyp-Hyp-Thr-Ala-Lys---,
(SEQ ID No: 99)
DOPA-Ala-Lys-Pro-Ser-DOPA-Hyp-Thr-DOPA-Hyp-Lys---,
or,
more preferably, one, or preferably both,
Z groups represent:
(SEQ ID No: 14)
Ala-Lys-Pro-Ser-Tyr-Hyp-Thr-Tyr-Hyp-Lys---,
or
(SEQ ID No: 15)
Ala-Lys-Pro-Ser-Tyr-Hyp-Thr-DOPA-Hyp-Lys---;

or, even more preferably, one, or preferably both, Z groups represent:

(SEQ ID No: 2)
Ala-Lys-Pro-Ser-Tyr-Hyp-Hyp-Thr-Tyr-Lys---,
or
(SEQ ID No: 1)
Ala-Lys-Pro-Ser-Tyr-Hyp-Hyp-Thr-DOPA-Lys---,

and
Q represents a Lys fragment.

Further compounds of the invention that comprise a peptide component of formula I as defined under (a) above that may be mentioned include those in which, in the peptide component of formula I:
A and B both represent A¹-Q¹-B¹;
A¹ and B¹ both represent Z;
one, or preferably both, Z groups represent:

(SEQ ID No: 14)
Ala-Lys-Pro-Ser-Tyr-Hyp-Thr-Tyr-Hyp-Lys---,
(SEQ ID No: 15)
Ala-Lys-Pro-Ser-Tyr-Hyp-Thr-DOPA-Hyp-Lys---,
(SEQ ID No: 38)
Lys-Ala-Lys-Pro-Ser-Tyr-Hyp-Hyp-Thr-DOPA---,
(SEQ ID No: 44)
HCA-Ala-Lys-Pro-Ser-Tyr-Hyp-Thr-Tyr-Hyp-Lys---,
(SEQ ID No: 45)
HCA-Ala-Lys-Pro-Ser-Tyr-Hyp-Hyp-Thr-Tyr-Lys---,
(SEQ ID No: 50)
DOPA-Ala-Lys-Pro-Ser-Tyr-Hyp-Thr-Tyr-Hyp-Lys---,
(SEQ ID No: 64)
Ala-Lys-Pro-Ser-DOPA-Hyp-Thr-DOPA-Hyp-Lys---,
(SEQ ID No: 65)
Ala-Lys-Pro-Ser-DOPA-Hyp-Hyp-Thr-DOPA-Lys---,
(SEQ ID No: 37)
Lys-Ala-Lys-Hyp-Ser-Tyr-Hyp-Hyp-Thr-DOPA---,
(SEQ ID No: 95)
HCA-Lys-Pro-Ser-DOPA-Hyp-Thr-Ala-Hyp-Lys---,
(SEQ ID No: 97)
DOPA-Lys-Pro-Ser-DOPA-Hyp-Thr-Ala-Hyp-Lys---,
(SEQ ID No: 99)
DOPA-Ala-Lys-Pro-Ser-DOPA-Hyp-Thr-DOPA-Hyp-Lys---,
or,
more preferably, one, or preferably
both, Z groups represent:
(SEQ ID No: 2)
Ala-Lys-Pro-Ser-Tyr-Hyp-Hyp-Thr-Tyr-Lys---
or
(SEQ ID No: 1)
Ala-Lys-Pro-Ser-Tyr-Hyp-Hyp-Thr-DOPA-Lys---;

and
Q¹ represents a Lys fragment.

Further compounds of the invention that comprise a peptide component of formula I as defined under (a) above that may be mentioned include those in which, in the peptide component of formula I:
A and B both represent A¹-Q¹-B¹;
A¹ and B¹ both represent A²-Q²-B²;
A² and B² both represent Z;
one, or preferably both, Z groups represent:

(SEQ ID No: 14)
Ala-Lys-Pro-Ser-Tyr-Hyp-Thr-Tyr-Hyp-Lys---,
(SEQ ID No: 15)
Ala-Lys-Pro-Ser-Tyr-Hyp-Thr-DOPA-Hyp-Lys---,
(SEQ ID No: 44)
HCA-Ala-Lys-Pro-Ser-Tyr-Hyp-Thr-Tyr-Hyp-Lys---,
(SEQ ID No: 50)
DOPA-Ala-Lys-Pro-Ser-Tyr-Hyp-Thr-Tyr-Hyp-Lys---,
(SEQ ID No: 65)
Ala-Lys-Pro-Ser-DOPA-Hyp-Hyp-Thr-DOPA-Lys---,
(SEQ ID No: 99)
DOPA-Ala-Lys-Pro-Ser-DOPA-Hyp-Thr-DOPA-Hyp-Lys---,

or, more preferably, one, or preferably both, Z groups represent:

(SEQ ID No: 51)
DOPA-Ala-Lys-Pro-Ser-Tyr-Hyp-Hyp-Thr-Tyr-Lys---,

or, even more preferably, one, or preferably both, Z groups represent:

(SEQ ID No: 2)
Ala-Lys-Pro-Ser-Tyr-Hyp-Hyp-Thr-Tyr-Lys---,
or
(SEQ ID No: 1)
Ala-Lys-Pro-Ser-Tyr-Hyp-Hyp-Thr-DOPA-Lys---,

and
Q¹ and Q² both represent Lys fragments.

Further compounds of the invention that comprise a peptide component of formula I as defined under (a) above that may be mentioned include those in which, in the peptide component of formula I:
A and B both represent A¹-Q¹-B¹;
A¹ and B¹ both represent A²-Q²-B²;
A² and B² both represent Z-Q³-Z;
one, or preferably both, Z groups represent:

(SEQ ID No: 2)
Ala-Lys-Pro-Ser-Tyr-Hyp-Hyp-Thr-Tyr-Lys---,
or
(SEQ ID No: 1)
Ala-Lys-Pro-Ser-Tyr-Hyp-Hyp-Thr-DOPA-Lys---;

and
Q¹, Q² and Q³ all represent Lys fragments.

Peptide components as defined under (c) above that may be Included in compounds of the invention that may be mentioned Include those of the amino acid sequence:

(SEQ ID NO: 114)
K-W²-Lys-X¹-Ser-U¹-X²-Y¹-I-J

wherein K represents an optional N-terminal HCA group;
W² may be absent (in which case Lys is the N-terminal amino add) or W² may represent a 1 or 2 amino add sequence, in which the amino adds are selected from one or more of the group Ser, Lys, Ala and DOPA;
Y¹ represents a single bond or a 1 to 3 (e.g. a 1 or 2) amino add sequence, in which the amino acids are selected from one or more of the group Lys, Ala, Pro, Hyp, diHyp, Thr, DOPA and Tyr;
I represents Pro, Hyp, diHyp, Thr, DOPA or Tyr;
J represents Lys or Is absent (In which case I represents the C-terminal amino acid); and
X¹, U¹ and X² are as hereinbefore defined.

When compounds of the invention comprise a peptide component of SEQ ID No: 114, those that may be mentioned Include those in which:
W² represents Ala or Ser, or is absent (in which case, Lys is the N-terminal amino acid);
X² represents Pro, Hyp or diHyp;
when K is not present, W² represents Ala or is absent and 3 represents Lys, then I represents Pro, Hyp, diHyp or Thr (i.e. I does not represent DOPA or Tyr).

Preferred compounds of the invention comprising a peptide component of SEQ ID No: 114 Include those in which:
U¹ represents DOPA or, more preferably Tyr;
X¹ represents Hyp or, more preferably, Pro;
X² represents diHyp or, more preferably, Hyp;
Y¹ represents a 3, a 1 or, preferably, a 2 amino acid sequence, in which the amino acids are selected from the group Pro, Hyp, Thr, DOPA and Tyr.

Peptide components of SEQ ID No: 114 that may be mentioned Include those in which W² represents Ser.

However, more preferred peptide components of SEQ ID No: 114 Include those in which W² is absent or, more preferably, W² represents Ala.

Preferred peptide components of SEQ ID No: 114 also Include those in which 3 represents Lys.

More preferably, peptide components of SEQ ID No: 114 also Include those in which I represents DOPA or Tyr, more preferably Pro or, especially, Hyp.

Preferred peptide components of SEQ ID No: 114 also Include those in which, when 3 represents Lys, I represents DOPA or Tyr, more preferably Pro or, especially, Hyp.

Preferred peptide components of SEQ ID No: 114 also Include those in which 3 is absent.

Preferred peptide components of SEQ ID No: 114 also include those in which, when 3 is absent, I represents DOPA or Tyr, more preferably Pro or, especially Hyp.

Further preferred peptide components of SEQ ID No: 114 Include those in which the amino acids in the sequence defined by Y¹ are selected from Lys, Pro, preferably DOPA, more preferably Hyp, Thr and Tyr.

Especially preferred peptide components of SEQ ID No: 114 Include those in which, in the sequence defined by Y¹:
the amino acid DOPA, preferably Thr or Lys or, more preferably, Tyr is linked to I;
the amino add Pro, or more preferably Hyp or Thr is linked to X².

Preferred values of Y¹ in peptide components of SEQ ID No: 114 above Include, when it is a 3-membered amino acid sequence, -Hyp-Thr-Tyr- or, more preferably -Hyp-Thr-DOPA-, -Thr-DOPA-Lys or -Thr-Tyr-Lys-, and, when it is a 2-membered amino acid sequence, -Thr-Tyr- or, more preferably, -Thr-DOPA-, -Pro-Thr- or, more preferably, -Hyp-Thr-.

Particular compounds of the invention comprising peptide components of SEQ ID No: 114 that may be mentioned Include those in which K is absent.

In this respect, peptide components of SEQ ID No: 114 Include those comprising the amino acid sequence:

(SEQ ID No: 115)
Lys-Pro-Ser-Tyr-Hyp-Hyp-Thr-DOPA-Hyp-Lys;
(SEQ ID No: 116)
Lys-Pro-Ser-Tyr-Hyp-Hyp-Thr-Tyr-Hyp-Lys;
(SEQ ID No: 117)
Lys-Pro-Ser-Tyr-Hyp-Thr-DOPA-Hyp-Lys;
(SEQ ID No: 118)
Lys-Pro-Ser-Tyr-Hyp-Thr-Tyr-Hyp-Lys;
(SEQ ID No: 119)
Ser-Lys-Pro-Ser-Tyr-Hyp-Hyp-Thr-DOPA-Lys;
(SEQ ID No: 120)
Ser-Lys-Pro-Ser-Tyr-Hyp-Hyp-Thr-Tyr-Lys;
(SEQ ID No: 121)
Ser-Lys-Pro-Ser-Tyr-Hyp-Thr-DOPA-Hyp-Lys;
(SEQ ID NO: 122)
Ser-Lys-Pro-Ser-Tyr-Hyp-Thr-Tyr-Hyp-Lys.

More preferred compounds of the invention comprising peptide components of SEQ ID No:
114 Include those comprising the amino acid sequence:

(SEQ ID No: 123)
Ala-Lys-Pro-Ser-Tyr-Hyp-Hyp-Thr-DOPA-Hyp-Lys;
(SEQ ID No: 124)
Ala-Lys-Pro-Ser-Tyr-Hyp-Hyp-Thr-Tyr-Hyp-Lys;

more preferably those
comprising the amino add sequence:

(SEQ ID No: 125)
Ala-Lys-Pro-Ser-Tyr-Hyp-Thr-DOPA-Hyp-Lys;

and
particularly those comprising the amino acid sequence:

(SEQ ID NO: 126)
Ala-Lys-Pro-Ser-Tyr-Hyp-Thr-Tyr-Hyp-Lys

Further compounds of the invention comprising peptide components of SEQ ID No: 114 that may be mentioned include those in which 3 is absent, such as those comprising the amino acid sequence:

(SEQ ID No: 127)
Lys-Pro-Ser-Tyr-Hyp-Hyp-Thr-DOPA-Hyp;
(SEQ ID No: 137)
Ala-Lys-Pro-Ser-Tyr-Hyp-Thr-Tyr-Hyp;
(SEQ ID No: 128)
Lys-Pro-Ser-Tyr-Hyp-Hyp-Thr-Tyr-Hyp;
(SEQ ID No: 129)
Lys-Pro-Ser-Tyr-Hyp-Thr-DOPA-Hyp;
(SEQ ID No: 130)
Lys-Pro-Ser-Tyr-Hyp-Thr-Tyr-Hyp;
(SEQ ID No: 131)
Ser-Lys-Pro-Ser-Tyr-Hyp-Hyp-Thr-Tyr;
(SEQ ID No: 132)
Ser-Lys-Pro-Ser-Tyr-Hyp-Thr-DOPA-Hyp;
(SEQ ID No: 133)
Ser-Lys-Pro-Ser-Tyr-Hyp-Thr-Tyr-Hyp;
(SEQ ID No: 134)
Ala-Lys-Pro-Ser-Tyr-Hyp-Hyp-Thr-DOPA-Hyp;
(SEQ ID No: 135)
Ala-Lys-Pro-Ser-Tyr-Hyp-Hyp-Thr-Tyr-Hyp;
(SEQ ID No: 136)
Ala-Lys-Pro-Ser-Tyr-Hyp-Thr-DOPA-Hyp;

and
particularly, those comprising the amino acid sequence:

(SEQ ID NO: 138)
Ser-Lys-Pro-Ser-Tyr-Hyp-Hyp-Thr-DOPA

Further compounds of the invention comprising peptide components of SEQ ID No: 114 include those in which K is an N-terminal HCA group, include those comprising the amino acid sequence:
HCA-Ala-Lys-Pro-Ser-Tyr-Hyp-Hyp-Thr-Tyr-Lys (SEQ ID No: 139); and, more preferably, that defined by the amino acid sequence:

(SEQ ID NO: 140)
HCA-Ala-Lys-Pro-Ser-Tyr-Hyp-Thr-Tyr-Hyp-Lys

Further preferred compounds of the invention comprising peptide components of SEQ ID No: 114 that may be mentioned include those in which W² is Ala and 3 is Lys, such as those comprising the amino add sequence:

(SEQ ID No: 144)
Ala-Lys-Hyp-Ser-DOPA-Hyp-Thr-DOPA-Hyp-Lys;
(SEQ ID No: 141)
Ala-Lys-Pro-Ser-DOPA-Hyp-Thr-DOPA-Hyp-Lys;
(SEQ ID No: 142)
Ala-Lys-Pro-Ser-Pro-Thr-Tyr-Pro-Lys;
(SEQ ID No: 143)
Ala-Lys-Hyp-Ser-Tyr-Hyp-Thr-Tyr-Hyp-Lys;

and
particularly, those defined by the amino acid sequence:

(SEQ ID NO: 145)
Ala-Lys-Hyp-Ser-Tyr-Hyp-Thr-DOPA-Hyp-Lys

Further preferred compounds of the invention comprising peptide components of SEQ ID No: 114 that may be mentioned include those in which 3 is absent, such as those comprising the amino acid sequence:

(SEQ ID No: 146)
HCA-Ala-Lys-Pro-Ser-Tyr-Hyp-Hyp-Thr-Tyr;
(SEQ ID No: 147)
HCA-Ala-Lys-Pro-Ser-Tyr-Hyp-Thr-Tyr-Hyp;
(SEQ ID No: 148)
Ala-Lys-Pro-Ser-DOPA-Hyp-Thr-DOPA-Hyp;
(SEQ ID No: 149)
Ala-Lys-Pro-Ser-Pro-Thr-Tyr-Pro;
(SEQ ID No: 150)
Ala-Lys-Hyp-Ser-Tyr-Hyp-Thr-Tyr-Hyp;
(SEQ ID No: 151)
Ala-Lys-Hyp-Ser-DOPA-Hyp-Thr-DOPA-Hyp;
(SEQ ID No: 152)
Ala-Lys-Hyp-Ser-Tyr-Hyp-Thr-DOPA-Hyp;
(SEQ ID No: 153)
Ala-Lys-Pro-Ser-DOPA-Hyp-Thr-Tyr-Lys-Hyp;
(SEQ ID No: 154)
Ala-Lys-Hyp-Ser-DOPA-Hyp-Thr-Tyr-Lys-Hyp;
(SEQ ID No: 155)
Ala-Lys-Pro-Ser-Tyr-Hyp-Thr-Tyr-Lys-Hyp;
(SEQ ID No: 156)
Ala-Lys-Hyp-Ser-Tyr-Hyp-Thr-Tyr-Lys-Hyp;
(SEQ ID No: 157)
Ala-Lys-Pro-Ser-Tyr-Hyp-Thr-DOPA-Lys-Hyp;
(SEQ ID No: 158)
Ala-Lys-Hyp-Ser-Tyr-Hyp-Thr-DOPA-Lys-Hyp;
(SEQ ID No: 159)
Ala-Lys-Pro-Ser-DOPA-Hyp-Thr-DOPA-Lys-Hyp;
and
(SEQ ID No: 160)
Ala-Lys-Hyp-Ser-DOPA-Hyp-Thr-DOPA-Lys-Hyp.

Other compounds of the Invention comprising peptide components of SEQ ID No: 114 that may be mentioned include those in which K and W² are both absent and Y¹ represents a single bond.

More preferred compounds of the Invention comprising peptide components of SEQ ID No: 114, In which K and W² are both absent and Y¹ represents a single bond, Include, in particular, those in which 3 represents Lys. Such peptide components are necessarily heptapeptide components of the amino acid sequence:

(SEQ ID NO: 161)
Lys-X¹-Ser-U¹-X²-I-Lys

wherein X¹, U¹, X² and I are as hereinbefore defined.

Preferred compounds of the Invention comprising peptide components of SEQ ID No: 161 Include those in which:
X¹ represents Hyp or, more preferably, Pro;
U¹ represents DOPA or, more preferably, Tyr;
X² represents Pro or, more preferably, Hyp.
I represents Hyp or, more preferably, DOPA or Tyr.

In this respect, preferred compounds of the Invention comprising peptide components of SEQ ID No: 161 Include those comprising the amino acid sequence:

(SEQ ID No: 162)
Lys-Pro-Ser-Tyr-Hyp-DOPA-Lys;
and
(SEQ ID NO: 163)
Lys-Pro-Ser-Tyr-Hyp-Tyr-Lys.

Particularly-preferred peptide sequences Include those comprising the amino acid sequence:

(SEQ ID No: 1)
Ala-Lys-Pro-Ser-Tyr-Hyp-Hyp-Thr-DOPA-Lys;
(SEQ ID No: 2)
Ala-Lys-Pro-Ser-Tyr-Hyp-Hyp-Thr-Tyr-Lys;
(SEQ ID No: 29)
Ala-Lys-Pro-Ser-Tyr-Hyp-Thr-Tyr-Hyp-Lys;
(SEQ ID No: 30)
Ala-Lys-Pro-Ser-Tyr-Hyp-Thr-DOPA-Hyp-Lys;
(SEQ ID No: 155)
Ala-Lys-Pro-Ser-Tyr-Hyp-Thr-Tyr-Lys-Hyp;
(SEQ ID No: 162)
Lys-Pro-Ser-Tyr-Hyp-DOPA-Lys;
and
(SEQ ID No: 163)
Lys-Pro-Ser-Tyr-Hyp-Tyr-Lys.

Peptide components as defined under (c) above that may be mentioned also Include those of the amino acid sequence:

(SEQ ID NO. 164)
Ala-Lys-Pro-Ser-Tyr-Hyp-Thr-Tyr-DOPA-Lys.

Particularly-preferred peptide sequences Include those comprising the amino acid sequence;

(SEQ ID No: 29)
Ala-Lys-Pro-Ser-Tyr-Hyp-Thr-Tyr-Hyp-Lys;
(SEQ ID No: 30)
Ala-Lys-Pro-Ser-Tyr-Hyp-Thr-DOPA-Hyp-Lys;
(SEQ ID No: 115)
Lys-Pro-Ser-Tyr-Hyp-Hyp-Thr-DOPA-Hyp-Lys;
(SEQ ID No: 116)
Lys-Pro-Ser-Tyr-Hyp-Hyp-Thr-Tyr-Hyp-Lys;
(SEQ ID No: 117)
Lys-Pro-Ser-Tyr-Hyp-Thr-DOPA-Hyp-Lys;
(SEQ ID No: 118)
Lys-Pro-Ser-Tyr-Hyp-Thr-Tyr-Hyp-Lys;
(SEQ ID No: 119)
Ser-Lys-Pro-Ser-Tyr-Hyp-Hyp-Thr-DOPA-Lys;
(SEQ ID No: 120)
Ser-Lys-Pro-Ser-Tyr-Hyp-Hyp-Thr-Tyr-Lys;
(SEQ ID No: 121)
Ser-Lys-Pro-Ser-Tyr-Hyp-Thr-DOPA-Hyp-Lys;
(SEQ ID No: 122)
Ser-Lys-Pro-Ser-Tyr-Hyp-Thr-Tyr-Hyp-Lys;
(SEQ ID No: 123)
Ala-Lys-Pro-Ser-Tyr-Hyp-Hyp-Thr-DOPA-Hyp-Lys;
(SEQ ID No: 124)
Ala-Lys-Pro-Ser-Tyr-Hyp-Hyp-Thr-Tyr-Hyp-Lys;
(SEQ ID NO. 164)
Ala-Lys-Pro-Ser-Tyr-Hyp-Thr-Tyr-DOPA-Lys.

Further particularly-preferred peptide sequences Include those comprising the amino acid sequence:

(SEQ ID No: 29)
Ala-Lys-Pro-Ser-Tyr-Hyp-Thr-Tyr-Hyp-Lys;
(SEQ ID No: 124)
Ala-Lys-Pro-Ser-Tyr-Hyp-Hyp-Thr-Tyr-Hyp-Lys;
(SEQ ID NO. 164)
Ala-Lys-Pro-Ser-Tyr-Hyp-Thr-Tyr-DOPA-Lys.

As used herein, Pro represents proline, Ala represents alanine, Ser represents serine, Tyr represents tyrosine, Hyp represents hydroxyproline (Including 3-hydroxyproline (3Hyp) and 4-hydroxyproline (4Hyp)), diHyp represents dihydroxyproline (including 3,4-dihydroxyproline (3,4diHyp), 3,5-dihydroxyproline (3,5diHyp) and 4,5-dihydroxyproline (4,5diHyp)), Thr represents threonine, Lys represents lysine, Ala represents alanine and DOPA represents 3,4-dihydroxyphenylalanine. 3,4-Dihydrocinnamic acid (HCA) residues are essentially DOPA residues but without the —NH₂ group in the 2- or α-carbon position relative to the carboxylic acid that is attached to the N-terminal amino acid (whether Lys or Ala).

Compounds of the invention, whether in the form of salts or otherwise, include regioisomers within amino acids of the peptides (for example diHyp, Hyp and Tyr moieties), as well as mixtures of such regioisomers. For example, included within the definition of Tyr are, not only tyrosine (4-hydroxyphenylalanine), but also 2- and 3-hydroxyphenylalanine. Included within the definition of Hyp are 4-hydroxyproline (4Hyp), 3-hydroxyproline (3Hyp) and 5-hydroxyproline (5Hyp). It is more preferred that Hyp residues are 4-hydroxyproline. Similarly, included within the definition of diHyp are 3,4-dihydroxyproline (3,4diHyp), 3,5-dihydroxyproline (3,5diHyp) and 4,5-dihydroxyproline (4,5diHyp). It is more preferred that diHyp residues are 3,4-dihydroxyproline (3,4diHyp).

Also, in addition to the standard central carbon atom of the amino acids in the compounds of the invention (which are normally but not exclusively in the L-configuration), certain amino acids in the sequence comprise further chiral carbon atoms. All such stereoisomers and mixtures (including racemic mixtures) thereof are included within the scope of the invention. In respect, included within the definition of Hyp are trans-4-hydroxy-L-proline, cis-4-hydroxy-L-proline, trans-3-hydroxy-L-proline, cis-3-hydroxy-L-proline, trans-5-hydroxy-L-proline and cis-5-hydroxy-L-proline, however we prefer that the Hyp that is employed in compounds of the invention is 4-hydroxy-L-proline. Similarly, corresponding definitions may be applied to diHyp, in which the two hydroxy groups can also be cis or trans relative to each other. In any event, individual enantiomers of peptide components as hereinbefore defined that may form part of a compound of the invention are included within the scope of the invention.

Compounds of the invention may be in the form of salts. Salts that may be mentioned Include pharmaceutically-acceptable salts, such as pharmaceutically-acceptable acid addition salts and base addition salts. Such salts may be formed by conventional means, for example by reaction of a compound of the invention with one or more equivalents of an appropriate acid or base, optionally in a solvent, or in a medium in which the salt is Insoluble, followed by removal of said solvent, or said medium, using standard techniques (e.g. In vacuo, by freeze-drying or by filtration). Salts may also be prepared by exchanging a counter-ion of the compound of the invention in the form of a salt with another counter-Ion, for example using a suitable Ion exchange resin.

Preferred salts Include, for example, acetate, hydrochloride, bisulfate, maleate, mesylate, tosylate, alkaline earth metal salts, such as calcium and magnesium, or alkali metal salts, such as sodium and potassium salts. Most preferably, compounds of the Invention may be in the form of acetate salts.

Compounds of the Invention may be prepared by way of conventional techniques. For example, compounds of the Invention (and particularly peptide components thereof may be prepared by way of standard amino acid coupling techniques, using standard coupling reagents and solvents, for example as described hereinafter. Compounds of the Invention may be synthesised from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed Include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

Compounds of the Invention that comprise radionuclides that are directly bonded to one or more peptides components may be prepared as described hereinafter. For example, radioactive Iodine atoms (such as ¹²³I, ¹²⁵I, ¹²⁹I or ¹³¹I) may be reacted with a peptide component of a compound of the Invention by way of direct Iodination of e.g. a tyrosine (or DOPA) residue in such a peptide components. Such a reaction may be carried out using chloramine-T, iodobeads or iodogen). Iodobeads and the iodogen may be used to minimize damage to the peptide component.

Chemical entities that are either capable of forming complexes with radionuclides as part of a labelling component (as described hereinbefore), or otherwise possess properties in their own right that are useful in the diagnosis and/or Imaging of cancers, may be coupled to free functional groups in peptide components of compounds of the Invention, either directly or using a linker moiety, for example as described hereinbefore or hereinafter.

Peptide components of compounds of the Invention may be conjugated or linked to such molecules (optionally via a linker) as part of a process to form a compound of the Invention, by electrostatically or covalently linking the relevant components to each other.

The term ‘electrostatic cross-linking’ will be understood by the skilled person to Include the association of disordered molecules Into an ordered state by virtue of Its nature or by electrostatic interactions (also referred to as ‘self-assembly’, which is a primary mechanism of gelation observed in amphiphilic peptide molecules (Hauser et al., Biomed. Mat. 2015, 11, 014103).

It is preferred however that compounds of the invention feature at least one covalent bond (e.g. an amide bond) formed by a reaction between either:
a carboxylic acid (i.e. —CO₂H) moiety present (e.g. at the C-terminus) of a peptide component as hereinbefore defined, and an amine (i.e. —NH₂) group that is present in a labelling component and/or in a linker moiety; and/or
an amine (i.e. —NH₂) moiety present (e.g. at the N-terminus) of a peptide component as hereinbefore defined, and a carboxylic acid (i.e. —CO₂H) group that is present in a labelling component and/or in a linker moiety.

Appropriate linker moieties that may be employed Include ACP.

Compounds of the invention may be Isolated from their reaction mixtures and, if necessary, purified using conventional techniques as known to those skilled in the art. Thus, processes for preparation of compounds of the invention as described herein may Include, as a final step, isolation and optionally purification of the compound of the invention.

It will be appreciated by those skilled in the art that, in the processes described above and hereinafter, the functional groups of intermediate compounds may need to be protected by protecting groups. The protection and deprotection of functional groups may take place before or after a reaction.

Protecting groups may be applied and removed in accordance with techniques that are well-known to those skilled in the art and as described hereinafter. For example, protected compounds/intermediates described herein may be converted chemically to unprotected compounds using standard deprotection techniques. The type of chemistry Involved will dictate the need, and type, of protecting groups as well as the sequence for accomplishing the synthesis. The use of protecting groups is fully described in ‘Protective Groups in Organic Synthesis’, 5th edition, T. W. Greene & P. G. M. Wutz, Wiley-Intersdenee (2014), the contents of which are Incorporated herein by reference.

Compounds of the invention are thus useful as human and animal medicine. They are therefore indicated as pharmaceuticals (and/or in veterinary science), although they may also be used as part of a medical device.

In particular, compounds of the invention are capable of targeting specific organs of the body, through the properties of the peptide component per se and/or properties that from the peptide components conjunction with the any one of the aforementioned labelling components.

Compounds of the invention may therefore be capable of associating with and/or binding to various cancers, malignant tumours, cancer cells and/or receptors thereof, in the body, through one or other of the properties of the peptide component, and/or those of any one of the aforementioned labelling components, including molecules that are capable of imaging cancer cells and/or tumours, by virtue of their physical properties, radionuclides, and/or chemical entities that are capable of forming complexes with radionuclides. Compounds of the invention may further comprise a targeting moiety that leads to internalization Into a cell of the compound of the invention or part thereof.

Compounds of the invention may further possess pharmacological activity in their own right and, in this respect, they may possess anticancer properties that may allow for the treatment of a cancer per se, that is treatment of a cancer by Interfering with the cancer or by treatment any of the symptoms of the cancer, such as pain and/or inflammation. Such anticancer properties may also Include the prevention of the onset of such a disease.

Particular cancers that may be mentioned include those that may benefit from targeted radionuclide therapy, such as thyroid cancer, cervical cancer, prostate cancer, breast cancer, brain tumours, esophageal cancers, lung cancer pancreatic cancers, skin cancers (such as basal cell carcinomas), blood cancers (including tumour regression in leukemia and refractory lymphoma), neuroendocrine tumours, other carcinomas (including squamous cell carcinoma and peritoneal carcinomas), metastases (including bone metastases), melanomas and solid tumours.

Particular radionuclides that may be mentioned that are useful in the therapeutic treatment of cancers are listed below.

¹¹⁷Lu and ⁹⁰Y may be used in peptide receptor radionuclide therapy and may be employed in therapy on small (e.g. endocrine, such as gastroenteropancreatic neuroendocrine) tumours, Including pancreatic ductal adenocardnoma; ²¹³BI may be employed for targeted alpha particle therapy in metastatic cancer; ¹⁶⁶Ho may be employed in the diagnosis and treatment of liver tumours; ¹⁹²Ir may be used as an internal radiotherapy source for cancer treatment; ⁶⁰Co may be employed in external beam radiotherapy (as a source of gamma radiation, regulating the direction and dose of radiation); ¹⁰³Pd may be employed in radiation therapy early stage prostate cancer and uveal melanoma; ²²³Ra may be employed in the treatment of metastatic bone cancer; ¹⁸⁸Re may be employed in pain relief in bone cancer; ¹⁵³Sm may be employed the treatment of bone metastases and in particular in relieving the pain of secondary cancers lodged in the bone, as well as in the treatment of prostate and breast cancer; and ⁸⁹Sr may be employed the treatment of bone metastases as well as in reducing the pain of prostate and bone cancer.

In particular, ¹³¹I is strong gamma ray emitter, but used for beta radiation therapy, and may thus be used to treat thyroid cancer, including thyroid carcinoma, and other malignant diseases. More particularly, ¹²⁵I may be used in cancer brachytherapy, such as in the prostate and the brain.

According to a further aspect of the invention, there is provided a method of treatment of cancer, which method comprises the administration of a compound of the invention or a salt thereof to a patient in need of such treatment.

In terms of radioactive Isotopes that may be employed in the imaging and/or diagnosis of cancers and/or tumours, ^{99m}Tc may be employed in various forms of medical imaging including in the imaging of tumours.

In addition, ²⁰¹TI may be used in non-specific tumour imaging, determining the location of low-grade lymphomas, thyroid tumour imaging; ¹¹C may be employed in brain tumour imaging; ¹⁸F may be employed in imaging of tumours (including prostate tumours), the myocardia and bones; and ⁶⁷Ga and ⁶⁸Ga may both be employed in tumour imaging, such imaging of neuroendocrine tumours and prostate cancers.

In particular, ¹³¹I may be used in imaging of the thyroid metastases and neuroectodermal tumours.

According to a further aspect of the invention, there is provided a method of imaging or diagnosing a cancer, which method comprises the administration of a compound of the invention or a salt thereof to a patient in need of such imaging and/or diagnosis, wherein the imaging is carried out using an appropriate nuclear medicine detection means.

Appropriate nuclear medicine detection means Include scintigraphy, SPECT and PET, as hereinbefore described.

Radioisotopes may also be employed in imaging of the body in non-oncology applications.

For example,Tc may be employed in various forms of medical imaging, including imaging of the skeleton and heart muscle in particular, but also the brain, thyroid, lungs (perfusion and ventilation), liver, spleen, kidney (structure and filtration rate), gall bladder, bone marrow, salivary and lacrimal glands, heart blood pool, infection and numerous specialised medical studies. ⁹⁹Mo is often used as a progenitor of

In addition, tritium (³H) may be used in the diagnosis of total body water; ¹¹C may be employed in parathyroid imaging; ¹⁴C may be employed in the detection of bacterial growth; ¹³N and ¹⁵O may both be employed in the imaging of myocardial blood flow; ¹⁵O may also be employed in imaging of cerebral blood flow; ²⁰¹Th may be used in the diagnosis of coronary artery disease other heart conditions such as heart muscle death; ⁶⁷Ga and ⁶⁸Ga may both be employed in the imaging of infections and inflammation; ⁸²Rb may be employed in positron emission tomography (PET) to identify myocardial ischemia (⁹²Sr is Its progenitor); ⁵¹Cr may be employed in the radiolabelling of red blood cells and in the quantification of gastrointestinal protein loss; ⁵⁷Co may be employed as a marker to estimate organ size and for in-vitro diagnostic kits. ⁵⁷Co and ⁵⁸Co may both be employed in gastrointestinal absorption investigations; ⁴⁷Ca may be employed in bone metabolism investigations; ⁶⁴Cu may be used to study genetic diseases affecting copper metabolism, such as Wilson's and Menke's diseases; ^{81m}Kr may be used in lung perfusion and/or ventilation imaging; ⁵⁹Fe may be used in studies of Iron metabolism in the spleen; ¹¹¹In may be employed in specialist diagnostic studies, such as brain studies, infection and colon transit studies; ⁴²K may be used for the determination of exchangeable potassium in coronary blood flow; ⁸²Ru may be employed in myocardial imaging; ⁷⁵Se may be employed in imaging of adrenal glands and in the investigation of bile salt absorption; ²²Na and ²⁴Na may both be used for studies of electrolytes within the body; and ¹³³Xe may be employed in the investigation of lung ventilation and cerebral blood flow.

In particular, ¹²³I may be employed in the diagnosis of thyroid function defects, renal imaging, neuroectodermal tumour imaging and imaging of Parkinson's Disease; ¹²⁵I may be used diagnostically to evaluate the filtration rate of kidneys and to diagnose deep vein thrombosis in the leg, it may also be employed in the diagnosis of abnormal liver function, renal (kidney) blood flow and urinary tract obstruction, as well Its use in radioimmunoassays to show the presence of hormones in tiny quantities; and ¹³¹I may be used to treat thyrotoxicosis and non-toxic goiter.

According to a further aspect of the invention, there is provided a method of medical imaging a body part, which method comprises the administration of a compound of the invention or a salt thereof comprising a radionuclide that Is relevant to the imaging of such a body part to a patient in need of such imaging, wherein the imaging is carried out using an appropriate nuclear medicine detection means, as hereinbefore defined.

The following radioisotopes may be used to treat the following non-oncological disorders ¹⁶⁵Dy may be used as an aggregated hydroxide for synovectomy treatment of arthritis; ¹⁶⁹Er may be used in the relief of arthritic pain in synovial joints; ³²P may be used in the treatment of polycythemia vera (excess red blood cells) and related disorders; and ¹⁸⁸Re may be employed to beta irradiate coronary arteries from an angioplasty balloon.

Compounds of the invention comprising one or more radionuclides may also be used to treat benign tumours (such as warts), and the early/initial stages of the development of cancers, including hyperplasia and pre-cancerous lesions, and may also be used to subject invasive organisms, such as viruses and bacteria, and infections caused thereby (including chlamydia, mycoplasma and the like), to radiation with a view to their eradication.

For those compounds of the invention that do not comprise one or more radionuclides, there is further provided a method of medical imaging (e.g. of internal areas of the body, a body part, and/or any associated physiology), which method comprises the administration of a compound of the invention or a salt thereof, which compound or salt thereof (preferably) does not comprise a radionuclide but comprises one or more labelling groups or components that is capable of medically imaging such parts of the body including vasculature networks in internal organs, including cancer cells and/or tumours, by virtue of one of more physical properties that it possesses, such as the ability to exhibit bioluminescence, and wherein the imaging is carried out using an appropriate medical imaging technique.

Appropriate medical imaging techniques Include radiography (projection radiography and fluoroscopy), tomography (including X-ray tomography and PET), function near-IR spectroscopy and magnetic particle imaging.

‘Patients’ Include reptilian, avian and, preferably, mammalian (particularly human) patients.

In accordance with the invention, compounds of the invention may administered locally, for example as part of brachytherapy or systemically, for example orally, Intravenously or intraarterially (including by intravascular and other perivascular devices/dosage forms (e.g. stents)), intramuscularly, cutaneously, subcutaneously, transmucosally (e.g. sublingually or buccally), intramucosally, rectally, intravaginally, intradermally, transdermally, nasally, pulmonarily (e.g. tracheally or bronchially), topically, or by any other parenteral route, in the form of a pharmaceutical preparation comprising the compound(s) in pharmaceutically acceptable dosage form(s).

Administration to the lower gastrointestinal tract may also be achieved by parenteral, and particularly by peroral, delivery, by means of standard delayed- or extended-release coating techniques known to those skilled in the art. In particular, distinct parts of the upper or lower intestine may be targeted. For example, colonic administration can also be achieved by way of colon-targeted drug delivery means that are initially administered perorally or parenterally.

Compounds of the invention may in particular be administered by direct parenteral administration, either systemically or locally to one or more organs of a patient.

Internal organs that may be mentioned Include the stomach, the intestines, the pancreas, the liver, the spleen, the cervix, the prostate, the bladder, the vascular system, the breast, the ovaries, the brain, the heart, the kidneys and the lungs.

In particular, when compounds of the invention/salts thereof are administered directly and parenterally, they may be administered intravenously, intraarterially, intravascularly, perivascularly, Intramuscularly, cutaneously, and/or subcutaneously, for example by way of direct injection, or by way of any other parenteral route, in the form of a compound of the invention or salt thereof in the form of a pharmaceutically-acceptable dosage form.

Pharmaceutically-acceptable formulations for use in injection (whether local (e.g. intradermally, intramucosally or subcutaneously) or systemic) may thus comprise compounds of the invention in admixture with a pharmaceutically-acceptable adjuvant, diluent or carrier, which may be selected with due regard to the Intended route of direct parenteral administration and standard pharmaceutical practice. Such pharmaceutically-acceptable carriers may be chemically Inert to the active compounds and may have no detrimental side effects or toxicity under the conditions of use. Such pharmaceutically-acceptable carriers may also Impart an Immediate, or a modified, release of the compound of the invention.

Suitable pharmaceutical formulations may be commercially available or otherwise prepared according to techniques that are described in the literature, for example, Remington the Science and Practice of Pharmacy, 22^{nd} edition, Pharmaceutical Press (2012) and Martindale—The Complete Drug Reference, 38th Edition, Pharmaceutical Press (2014) and the documents referred to therein, the relevant disclosures in all of which documents are hereby Incorporated by reference. Otherwise, the preparation of suitable formulations including compounds of the invention may be achieved non-inventively by the skilled person using routine techniques.

Formulations for injection (whether for systemic, intradermal, intramucosal, subcutaneous and/or Intramuscular administration, or otherwise) may thus be in the form of an aqueous formulation such as an a suspension and/or, more preferably a solution (e.g. an (optionally) buffered aqueous formulation (e.g. solution), such as a physiological saline-containing formulation (e.g. solution), a phosphate-containing formulation (e.g. solution), an acetate-containing formulation (e.g. solution) or a borate-containing formulation (e.g. solution), or a freeze-dried powder that may be reconstituted with a vehicle, such as an aqueous vehicle prior to use (e.g. injection)).

Formulations for injection may Include other suitable excipients known to those skilled in the art, such as solvents (e.g. water), co-solvents, solubilizing agents (e.g. cyclodextrins), wetting agents, suspending agents, emulsifying agents, thickening agents, chelating agents, antioxidants, reducing agents, antimicrobial preservatives, bulking agents and/or protectants.

Formulations for injection are preferably buffered by standard techniques to physiologically-acceptable pH values (e.g. pHs of between about 4.5 and about 9.5, e.g. about 6 and about 9, such as between about 6.5 and about 8.5) using buffers and/or pH modifiers as described herein, and/or may further comprise tonicity-modifying agents (such as sodium chloride).

Administration by injection is also useful for administering the compounds of the invention, in the form of a solution of suspension Into e.g. the dermis (e.g. intradermal or subcutaneous injection), the mucosa (e.g. intramucosal injection), a joint cavity or the eyes.

Compounds of the Invention may further and/or In the alternative be combined with appropriate excipients to prepare:
gel formulations (for which suitable gel matrix materials Include cellulose derivatives, carbomer and alginates, gummi tragacanthae, gelatin, pectin, carrageenan, gellan gum, starch, Xanthan gum, cationic guar gum, agar, noncellulosic polysaccharides, saccharides such as glucose, glycerin, propanediol, vinyl polymers, acrylic resins, polyvinyl alcohol, carboxyvinyl polymer and, particularly, hyaluronic acid);
lotions (for which suitable matrix materials Include cellulose derivatives, glycerin, noncellulosic polysaccharides, polyethylene glycols of different molecular weights and propanediol);
pastes or ointments (for which suitable paste matrix materials Include glycerin, paraffin, polyethylene glycols of different molecular weights, etc.);
creams or foams (for which suitable excipients (e.g. foaming agents) Include hydroxypropyl methyl cellulose, gelatin, polyethylene glycols of different molecular weights, sodium dodecyl sulfate, sodium fatty alcohol polyoxyethylene ether sulfonate, corn gluten powder and acrylamide);
powder aerosols (for which suitable excipients Include mannitol, glycine, dextrin, dextrose, sucrose, lactose, sorbitol and polysorbates, e.g. a dry powder Inhalant);
liquid, for example, water (aerosol) sprays for oral use or for Inhalation (for which suitable excipients Include viscosity modifiers, such as hyaluronic acid, sugars, such as glucose and lactose, emulsifiers, buffering agents, alcohols, water, preservatives, sweeteners, flavours, etc.); and/or
injectable solutions or suspensions (which may be aqueous or otherwise and for which suitable excipients Include solvents and co-solvents, solubilizing agents, wetting agents, suspending agents, emulsifying agents, thickening agents, chelating agents, antioxidants, reducing agents, antimicrobial preservatives, buffers and/or pH modifiers, bulking agents, protectants and tonicity-modifying agents), particular Injectable solutions or suspensions that may be mentioned Include dermal fillers (i.e. Injectable fillers or soft-tissue fillers), particularly when the compound of the Invention Is combined with hyaluronic acid.

According to a further aspect of the Invention there Is provided a process for the preparation of a pharmaceutical composition/formulation, as defined herein, which process comprises bringing Into association a compound of the Invention, as hereinbefore defined, with one or more pharmaceutically-acceptable excipient, as hereinbefore defined.

According to a further aspect of the Invention there Is provided a (e.g. pharmaceutical) composition comprising a compound of the Invention and one or more pharmaceutically-acceptable excipient, such as an adjuvant, diluent or carrier.

Administration of the compounds of the Invention may be continuous or Intermittent. The mode of administration may also be determined by the timing and frequency of administration, but is also dependent, in the case of therapeutic uses, on the severity of the condition.

Depending on the disorder, and the patient, to be treated, as well as the route of administration, compounds of the invention may be administered at varying therapeutically effective doses to a patient in need thereof.

Similarly, the amount of the compound of the invention in a formulation will depend (in the case of therapeutic treatment) on the severity of the condition, and on the patient, to be treated, but may be determined by the skilled person.

In any event, the medical practitioner, or other skilled person, will be able to determine routinely the actual dosage, which will be most suitable for an individual patient, depending on the route of administration. The dosages mentioned herein are exemplary of the average case; there can, of course, be individual Instances where higher or lower dosage ranges are merited, and such are within the scope of this invention.

Doses may be administered between once and four (e.g. three) times daily.

Appropriate concentrations of compounds of the invention in an aqueous solution product may be about 0.01 (e.g. about 0.1) to about 15.0 mg/mL, in all cases calculated as the free (non-salt) compound.

In any event, the dose administered to a mammal, particularly a human, in the context of the present invention should be sufficient to effect a therapeutic (or diagnostic) response in the mammal over a reasonable timeframe (as described hereinbefore). One skilled in the art will recognize that the selection of the exact dose and composition and the most appropriate delivery regimen will also be Influenced by inter aa the pharmacological properties of the formulation, and the physical condition and mental acuity of the recipient, as well as the age, condition, body weight, sex and response of the patient to be treated, as well as genetic differences between patients.

Wherever the word ‘about’ is employed herein, for example in the context of amounts, such as concentrations and/or doses of the compounds of the Invention, molecular weights or pHs, it will be appreciated that such variables are approximate and as such may vary by ±10%, for example ±5% and preferably ±2% (e.g. ±1%) from the numbers specified herein. In this respect, the term ‘about 10%’ means e.g. ±10% about the number 10, i.e. between 9% and 11%.

The compounds uses and methods described herein may also have the advantage that, in imaging of body parts, and in the diagnosis and/or the treatment of the conditions mentioned hereinbefore, they may be more convenient for the physician and/or patient than, be more efficacious than, be less toxic than, have a broader range of activity than, be more potent than, produce fewer side effects than, or that it/they may have other useful pharmacological properties over, similar compounds or methods (treatments) known in the prior art, whether for the same such use or otherwise.

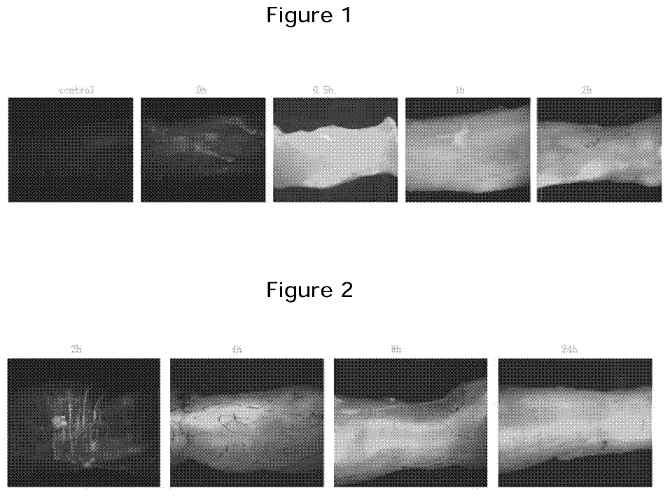

The invention is illustrated, but in no way limited, by the following examples with reference to the figures, in which FIGS. 1 and 2 show, in in vitro and ex vivo experiments, respectively, fluorescence in rat rectum samples at different time points following the administration of a fluorescein-labelled peptide according to the invention.

### EXAMPLES

### Example 1

Fluorescein-Labelled
(incorporating SEQ ID No: 30)
[Ala-Lys-Pro-Ser-Tyr-Hyp-Thr-DOPA-Hyp-Lys)₂-
Lys]₂-Lys

(a) [(ACP-Ala-Lys-Pro-Ser-Tyr-Hyp-Thr-DOPA-Hyp-Lys)₂-Lys]₂-Lys

Fmoc-Lys(fmoc)-Wang resin (2.5 g, GL Blochem, Shanghai, China) was loaded Into a glass reaction column.

Methylene chloride (DCM, 50 mL; Shandong Jinling Chemical Industry Co. Ltd., Shandong, China) was added to the column and allowed to soak the resin for about half an hour. The DCM was then removed by vacuum filtration.

The resin was washed 3 times with N,N-dimethylformamide (DMF, 50 mL; Shandong Shitaifeng Fertilizer Industry Co Ltd, Shandong, China).

A 20% piperidine solution in DMF (50 mL; Shandong Shitaifeng Fertilizer Industry Co. Ltd, Shandong, China) was added as deprotection solution and reacted for 20 minutes. The solution was then removed by vacuum filtration and the resin in column was washed with DMF six times.

Fmoc-Lys(fmoc)-OH (1.33 g; 36810, GL Blochem, Shanghai, China) and 2-(1H-benzotriazole-1-yl)-1,1,3,3-tetramethylaminium tetrafluoroborate (TBTU, 0.72 g; GL Blochem) were added to the resin. DMF (50 mL) was added to the reaction column, followed by N,N-diisopropylethylamine (DIPEA, 0.58 g; Suzhou Highfine Biotech Co. Ltd, Jiangsu, China). A Kaiser Test was carried out with few of the resin after 30 minutes reaction, a yellow colour of the solution and colourless gel indicating the reaction was complete. The solvent was removed by vacuum filtration.

The above coupling steps were repeated to couple the remaining amino acids, the amounts of the amino adds and the condensation agents (TBTU, DIPEA) were doubled (by mols) compared to the fust coupling step described above: Fmoc-Lys(Boc)-OH, Fmoc-4-Hyp(tBu)-OH, Fmoc-Dopa(Acetonide)-OH, Fmoc-Thr(tBu)-OH, Fmoc-4-Hyp(tBu)-OH, Fmoc-Tyr(tBu)-OH, Fmoc-Ser(tBu)-OH, Fmoc-Pro-OH, Fmoc-Lys(Boc)-OH, Fmoc-Ala-OH and Fmoc-ACP-OH.

After Fmoc-ACP-OH was coupled on the resin, a deprotection step was carried out to remove the Fmoc protection on ACP. The resin was washed 3 times with DMF (100 mL each time). A 20% piperldine solution in DMF (100 mL) was added as a deprotection solution and reacted for 20 minutes and then removed by vacuum. Then the resin was washed with DMF for six times.

(b)[5-FITC-ACP-Ala-Lys-Pro-Ser-Tyr-Hyp-Thr-DOPA-Hyp-Lys)2-Lys]2-Lys

Fluorescein isothiocyanate Isomer I (5-FITC, 1.75 g; F809535, Macklin Biochemical Co. Ltd., Shanghai, China) was added to the resin. DMF (150 mL) was added to the reaction column, followed by N,N-diisopropylethylamine (DIPEA, 1.17 g; Suzhou Highfine Biotech Co. Ltd, Jiangsu, China). A Kaiser Test was carried out with few of the resin after 16 hours reaction, a yellow color of the solution indicating the reaction was complete. The solvent was removed by vacuum filtration.

Then, the resin was washed three times each with the following solvents, DMF (150 mL each time), DCM (150 mL each time) and methanol (150 mL each time; Xilong Scientific Co., Ltd., Guangdong, China). The resin was dried under vacuum for about 2 hours.

100.0 mL (i.e. 10 mL per gram of the dried resin) of lysate, which comprised of 95% trifluoroacetic acid (TFA), 2.5% water and 2.5% trilsopropylsilane (Tls), were added to Immerse the resin-bounded peptide-containing compound. After cleavage for about 2 hours, the solid support was removed by filtration and the filtrate was collected under reduced pressure. The filtrate was precipitated with 1000 mL (i.e. 10 mL per ml of the filtrate) of diethyl ether (Xilong Scientific Co., Ltd., Guangdong, China) and the sediment was collected by filtration. The sediment was dried by vacuum for about 2 hours, yielding 7.21 g of crude title compound.

The crude product was firstly analyzed as a 1 mg/mL sample in pure water and detected using a Shimadzu LCMS-8050 system. The analysis column was an Agilent ZORBAX Eclipse SB-C18 (4.6×250 mm, 5 μm column; detection: UV at 220 nm; solvent A: 0.1% TFA in MeCN, solvent B: 0.1% TFA in water, with a linear gradient from 5%˜90% solvent A concentration in 50 minutes; flow rate 1.0 ml/min; sample volume: 10 NL).

The target peak was eluted at 20.253 minutes and had the expected molecular weight, with a purity of 37.643%.

MS: m/z 7131.2

7.2 g of crude product was then dissolved in 50 mL of pure water and purified using LC3000 semi-preparation equipment. The preparation column model was a Dubhe-C18 model (Hanbon Sci. & Tech. Co., Ltd., Jiangsu, China) (50*250 mm, 100A column; detection: LN at 220 nm). The appropriate gradient for elution was calculated from LCMS detection step (Solvent A: 0.1% TFA in MeCN, solvent B: 0.1% TFA in water, with a linear gradient from 15%˜35% solvent A concentration in 30 minutes; flow rate 60.0 ml/mm;). Fractions were collected and analyzed using a Shimadzu LC-20 HPLC system (column as above, except with a linear gradient from 30%-55% solvent A concentration in 25 minutes).

Fractions with a purity of 98% were then mixed together for an anion exchange step. This was achieved using a LC3000 semi-preparation equipment (preparation column model: Dubhe-C18 model (as above). The fractions were diluted one time with pure water and loaded to the column directly, after that the column was washed with 0.37% of ammonium acetate in pure water for about 20 minutes followed by pure water for another 20 minutes at the flow rate of 60 ml/min, then eluted with the following gradient (Solvent A: 0.1% HAc in MeCN, solvent B: 0.1% HAc in water, with a linear gradient from 15%˜35% solvent A concentration in 30 minutes; flow rate 60.0 ml/min). Fractions were collected and analyzed using Shlmadzu LC-20 HPLC system (column and conditions as above). Fractions with a purity of 98% were mixed and freeze-dried to give 1.21 g of the purified title compound.

### Example 2

In Vivo and Ex Vivo Adhesive Study

The title compound of Example 1 above was dissolved in distilled water to make a 0.05 mg/mL solution. The fluorescence absorption of the solution was scanned at 520 nm using a Hitachi F-7000 fluorescence spectrophotometer with an Interval of 20 nm. The optimal excitation wavelength was 450 nm and the maximum absorption wavelength was 521 nm.

The fluorescence intensity did not change after one hour at room temperature, which indicated that the test compound was stable.

In an in vivo study, four rats were anesthetized by intraperitoneal injection of 5% chloral hydrate at a dose of 7 ml/kg. 1 mL of the solution of test compound was Injected Into the rectum using a 2 mL syringe. Swallowtail clamps were used to seal the anus to keep the liquid in rectum for about 2 hours. Rats were sacrificed at 2 hours, 4 hours, 8 hours and 24 hours after injection of test compound.

In a separate ex vivo study, rats were anesthetized and their abdominal cavities opened. 8 cm of the rectum was removed and cut Into 2 pieces, with fat and contents stripped. 1 mL of normal saline was used to flush the rectal cavities, which were then placed Into a constant humidity plate. 1 mL of the solution of test compound was Injected Into the rectum and ligated at both ends. Samples were taken at 0 hours, 0.5 hours, 1 hour and 2 hours for fluorescence imaging. A MZX81 fluorescence stereomicroscope was used for imaging (Guangzhou Micro-Shot Technology Co., Ltd).

The fluorescence Images are show in FIG. 1 (in vivo study) and FIG. 2 (ex vivo study). Both sets of results show that the adhesiveness of test compound (and particularly the peptide component thereof) to mucous was very stable. In the in vivo samples in particular, the fluorescence intensity did not decrease significantly even with constant fecal excretion.

This results indicate that compound of the invention can be used as tracer in metabolism studies.

### Example 3

³H-Labelled
(incorporating SEQ ID No: 30)
[(Ala-Lys-Pro-Ser-Tyr-Hyp-Thr-DOPA-Hyp-Lys)₂-
Lys]₂-Lys

The subtitle compound was prepared essentially as described in Example 1(a) above, except that Fmoc-Ala-OH was the last amino acid to be added.

The target peak was eluted at 11.589 minutes and had the expected molecular weight (MS: m/z 5127.2).

(c)³H-Labelled [(Ala-Lys-Pro-Ser-Tyr-Hyp-Thr-DOPA-Hyp-Lys)₂-Lys]₂-Lys

5 bottles (1 mCi/mL, 2 mL/bottle) of [³H] succinimide acrylate were combined in a 15 mL centrifuge tube, dried with nitrogen. 50 μL of DMSO was added followed by 1 mL of 50 mM pH 8.0 boric acid borax buffer-50 mM NaCl. Dissolution occurred in a vortex.

[(Ala-Lys-Pro-Ser-Tyr-Hyp-Thr-DOPA-Hyp-Lys)2-Lys]2-Lys (see step (a) above; 20.4 mg) 20 was added to the mixture with shaking for 1 hour at 25° C. and 200 rpm. 0.50 mL (1 mmol/L) of hydroxylamine was then added with shaking for 3 minutes.

The reaction solution was then transferred Into a centrifugal ultrafiltration tube (30000 NMWL), and centrifuged at 4000×g for 10 minutes. The supernatant was collected Into a 20 mL glass bottle, eluted with acetic acid solution at pH 5.0 6 times, until the radioactivity was less than 1% of the initial reading.

### Example 4

Pharmacokinetic Study Tritium-Labelled Peptide

1 mL of a pH 5.0 acetic acid solution was added to the product of Example 3 above with even mixing. 0.952 mL of the resultant solution was added to a 40 mL glass bottle.

About 200 μL of 0.5% Evans Blue aqueous solution was added followed by even mixing. 20.0088 g of [(Ala-Lys-Pro-Ser-Tyr-Hyp-Thr-DOPA-Hyp-Lys)2-Lys]2-Lys (see Example 3(a) above) was added to the resultant mixture and mixed evenly in the 40 mL glass bottle to obtain a gel comprising the compound of the invention. 30 Sprague-Dawley rats, half male and half female (Beijing Vital River Laboratory Animal Technology Co., Ltd.) were quarantined and acclimatised in polycarbonate cages for 5 to 7 days. The environmental conditions were controlled at room temperature of between 20° C. and 26° C., with a relative humidity of between 40% and 70%, with 12 hours of alternating lightness and darkness.

To obtain groups that were comparable by body weight of the same sex, all rats will be randomly assigned to respective treatment groups. The body weights required for randomization were obtained on Day 1 (before dosing). After randomization, rats were assigned to one of five groups (three animals per sex in each group).

After weighing, animals were anesthetized by Intramuscular Injection of Zoletil-50. The animals were placed on their backs. Stools at the end of their rectums were gently squeezed out, and the skin around anus was disinfected with 75% alcohol.

A closed catheter (No. 8, double lumen with guide wire) was Inserted Into the anus of the rats to a depth of about 3 cm, and then a gastric lavage needle was slowly Inserted to a depth of at least 3.5 cm. After lifting the perlanal skin, fixing the catheter and the gavage needle, about 1 mL of water was Injected Into the bladder cavity of the syringe guide catheter to make it expand. Then the gel comprising the title compound was given quickly through the needle.

After administration, the catheter was ligated and fixed by wrapping tape around the root of the rats' tails. The catheter remained in the rectum for about 4 hours before removal.

Blood, rectal contents and rectal mucosa samples were collected. All centrifuge tubes coated with EDTA-K2 were stored either in a refrigerator (at 2 to 8° C.) or In a cooler filled with Ice, and were protected from light prior to use. The collected blood was transferred Into the centrifuge tubes and stored in an Ice box protected from light after being mixed manually by reversing the tubes at least 5 times.

Then, the tubes were centrifuged at 1800 g for 10 minutes at between 2 and 8° C., 2 hours after blood collection. After centrifugation, the collected plasma samples were transferred Into newly-labeled centrifuge tubes and aliquoted Into two sets, stored ay below −70° C.

Plasma, rectal contents and rectal mucosa samples were analyzed using a liquid scintillation counter, and the radioactive concentration was calculated according to the actual sampling volume. Half-lives were also calculated using WinNonlin software. MS Excel was used for data statistical analysis, including mean, standard deviation (SD), and coefficient of variation (CV), etc.

The detailed pharmacokinetics parameters were showed in Table 1 below.

TABLE 1

| | Total radioactivity (ng Eq./g) | | | | |
|---|---|---|---|---|---|
| Matrix | 1 h | 6 h | 24 h | 72 h | 168 h (Cₘₐₓ %) |
| Rectal | 35322 ± 515 | 21981 ± 153 | 56.4 ± 54. | 44.3 ± 27. | 16.5 ± 6.88(0.05% |
| Rectal | 53593 ± 472 | 11163 ± 702 | 339 ± 95. | 147 ± 84. | 37.6 ± 10.2(0.07% |
| Plasma | 47.8 ± 12.2 | 54.1 ± 14.4 | 18.6 ± 6.2 | 15.2 ± 8.4 | 13.3 ± 3.41(24.58 |

Total radioactivity unit (ng Eq./g)=DPM/g÷S.A. ×1000, S.A.=144,300 DPM/μg

After rectal administration of 1.5 mg/100 μCi/kg of the test compound, there was no abnormality during the whole experiment, and there was no obvious adverse reaction after administration. This suggested that the experimental dose of tritiated test compound is well tolerated in rats.

After rectal administration of 1.5 mg/100 μCi/kg of the test compound to male and female rats, the concentrations of total radioactivity in rectal mucosa, rectal contents and plasma were similar between males and females, indicating that there were no significant gender differences.

The radioactivity concentration of the rectal mucosa was the highest at one hour after rectal administration, with an average concentration of 53593 ng Eq./g, 1121 times higher than that of plasma (47.8 ng Eq/g) at the same time. The radioactivity concentration of rectal contents (35322 ng Eq./g) was lower than that of rectal mucosa, which was related to the dilution of sample concentration by the flushing operation.

The average radioactivity concentration of the rectal mucosa decreased to 11163 ng Eq./g, about 20.83% of that of ¹H, which was still significantly higher than that of plasma (54.1 ng Eq./g) at the same time point. At 24 hours and at 72 hours after administration, there was still a high level of radioactivity in the rectal mucosa, with average concentrations of 339 ng Eq./g and 147 ng Eq./g, which were about 6.01 and 3.32 times of that in the rectal contents at the same time point, respectively.

A small amount of radioactivity could be detected in the rectal mucosa and rectal contents at 168 hours after the last collection. The radioactivity concentration in rectal mucosa (37.6 ng Eq./g) was 2.28 times higher than that in rectal contents (16.5 ng Eq./g). The half-life of total radioactivity elimination of the rectal mucosa, rectal contents and plasma were 45.8 hours, 79.3 hours and 316 hours, respectively.

In summary, after a single rectal administration of 1.5 mg/100 p Cl/kg of test compound in male and female SD rats, the total radioactivity in plasma was much lower than that in the local area of Interest. The total radioactivity in rectal mucosa and Its contents was the highest 1 hour after administration, and then the radioactivity was rapidly eliminated. The average concentration at 24 hours was about 0.63% and 0.16% of C-mx respectively, and then elimination was slow. At 168 hours after the last collection, the radioactivity in rectal mucosa and rectum decreased. A small amount of radioactivity could still be detected in the contents, Indicating the long-term retention of radioactivity in the local administration.

### Example 5

¹²⁵I-Labelled [(Ala-Lys-Pro-Ser-Tyr-Hyp-Thr-DOPA-Hyp-Lys)₂-Lys]₂-Lys (incorporating SEQ ID No: 30)

The aim of this study was to determine the stability of compound of the Invention in vivo after being polymerized on the surface of bone particles. Bone particles were first stained with Coomassie brilliant Blue for easy spotting in the tissue after Injection and in vivo Incubation.

2 g of bone beads were stained with colloidal Commassie blue staining solution for 1 hour at room temperature. The beads were washed with 25% methanol in water for 30 minutes and then with 5×PBS until the supernatant was clear.

500 μL of 10.2 mg/mL of the compound of Example 1(a) above at pH 4.8 was mixed with 300 μL of 100 mM acetic acid-acetate buffer at pH 5.0 and 5 μL of radiolabelled Iodine, which was then left at room temperature for 5 minutes. Two Iodobeads® coated with Iodogen® (1,3,4,6-tetrachloro-3α,6α-diphenylglycouril, Pierce) were added and left at room temperature in a fume hood for 15 minutes.

The resultant mixture was then transferred directly to a PD10 column (GE Healthcare) pre-equilibrated with 100 mM acetic acid-acetate buffer. The buffer was added dropwise until radioactivity started to appear at the end of column.

Five drops per tube were collected in individual tubes until the radioactivity was down to almost baseline levels. The most highly radioactive aliquots were pooled labelled in a gamma counter.

800 μL of the ¹²⁵I-labeled peptide from the most highly radioactive aliquots were mixed with 4 mL of the same peptide that was not labelled for coating experiments at a concentration of approximately 10 mg/mL.

24 mL of acetic acid-acetate buffer and 2 g of pre-stained bone beads were added Into the above mixture. 1 M Tris (pH 7.9) was added dropwise under constant agitation until the pH reached 7.5. Then resultant was then kept at 4° C. overnight to coat the beads.

The beads were then spun down to remove the supernatant, washed with 2×40 mL of cold PBS. Radioactivity of the beads was measured using a gamma counter.

The beads were re-suspended in cold PBS and then divided Into 65 Eppendorf tubes to be individually taken up Into injection syringes.

This is followed by injection Into animals as a tracer for detection.

### Example 6

Chloramine-T Method for Radiolabeling Using ¹²⁵I

Chloramine-T (p-toluene sulfonochloramine) is an effective method of labelling a variety of proteins and peptides. This oxidative method Involves exposure of the substrate to Chloramine-T in the presence of NaI, ¹²⁵I.. or ¹³¹I-, for a short time period and produces high specific activity proteins or peptides labeled with carrier-free radioiodine, resulting in substitution of ¹²⁵I or ¹³¹I into benzene rings of tyrosine (or DOPA) residues.

5 μg of the peptide of Example 1 above is dissolved in 10 μL of pure water. 30 μL of 0.5M phosphate buffer at pH 7.4 is then added with thorough mixing. An amount (such as about 74 MBq/10 μL) of Na¹²⁵I solution is then added with thorough mixing.

100 μg of Chloramine-T In 10 μL of pure water Is then added quickly with mixing. The 35 mixture is allowed to react at room temperature for about 1 to 3 minutes, and is then quenched by adding 0.2 mL of a solution of 200 μg of Na₂S₂O₅ in water.

Finally, the mixture is desalted on a Sephadex G50 column, to yield the title compound.

### Example 7

(SEQ ID NO: 29)
Ala-Lys-Pro-Ser-Tyr-Hyp-Thr-Tyr-Hyp-Lys

Fmoc-Lys(Boc)-Wang resin (9.15 g, 41301, GL Biochem, Shanghai, China) was loaded Into a glass reaction column.

Methylene chloride (DCM, 200 mL; Shandong Jinling Chemical Industry Co. Ltd., Shandong, China) was added to the column and allowed to soak the resin for about half an hour. The DCM was then removed by vacuum filtration.

The resin was washed 3 times with N,N-dimethylformamide (DMF, 200 mL; Shandong Shitaifeng Fertilizer Industry Co Ltd, Shandong, China).

A 20% piperidine solution in DMF (200 mL; Shandong Shitaifeng Fertilizer Industry Co Ltd, Shandong, China) was added as deprotection solution and reacted for 20 minutes. The solution was then removed by vacuum filtration and the resin in column was washed with DMF six times.

Fmoc-4-Hyp(tBu)-OH (3.68 g; 21303, GL Blochem, Shanghai, China) and 2-(1H-benzotriazole-1-yl)-1,1,3,3-tetramethylaminium tetrafluoroborate (TBTU, 2.89 g; 00705, GL Biochem, Shanghai, China) were added to the resin. DMF (150 mL) was added to the reaction column, followed by N,N-diisopropylethylamine (DIPEA, 2.33 g; Suzhou Highfine Biotech Co. Ltd, Jiangsu, China). A Kaiser Test was carried out with few of the resin after 30 minutes reaction, a yellow color of the solution and colorless gel indicating the reaction was complete. The solvent was removed by vacuum filtration.

The above coupling steps were repeated to couple the remaining amino acids in the same amounts (by mols): Fmoc-Tyr(tBu)-OH, Fmoc-Thr(tBu)-OH, Fmoc-4-Hyp(tBu)-OH, Fmoc-Tyr(tBu)-OH, Fmoc-Ser(tBu)-OH, Fmoc-Pro-OH, Fmoc-Lys(Boc)-OH and Fmoc-Ala-OH.

In a separate procedure, after Fmoc-Ala-OH was coupled on the resin, a deprotection step was carried out to remove the Fmoc protection on DOPA. The resin was washed 3 times with DMF (200 mL each time). A 20% piperidine solution in DMF (200 mL) was added as a deprotection solution and reacted for 20 minutes. Then, the resin was washed three times each with the following solvents, DMF (200 mL each time), DCM (200 mL each time) and methanol (200 mL each time; Xilong Scientific Co., Ltd., Guangdong, China). The resin was dried under vacuum for about 2 hours.

130.0 mL (i.e. 10 mL per gram of the dried resin) of lysate, which comprised of 95% trifluoroacetic acid (TFA), 2.5% water and 2.5% triisopropylsilane (Tls), were added to Immerse the resin-bounded peptide-containing compound. After cleavage for about 2 hours, the solid support was removed by filtration and the filtrate was collected under reduced pressure. The filtrate was precipitated with 1300 mL (i.e. 10 mL per ml of the filtrate) of diethyl ether (Xilong Scientific Co., Ltd., Guangdong, China) and the sediment was collected by filtration. The sediment was dried by vacuum for about 2 hours, yielding 4.13 g of crude title compound.

The crude product was firstly analyzed as a 1 mg/mL sample in pure water and detected using a Shimadzu LCMS-8050 system (Shimadzu Corporation, Kyoto, Japan). The analysis column was an Agilent ZORBAX Eclipse SB-C18 (4.6×250 mm, 5 μm) column; detection: UV at 220 nm; solvent A: 0.1% TFA in MeCN, solvent B: 0.1% TFA in water, with a linear gradient from 5%-90% solvent A concentration in 50 minutes; flow rate 1.0 ml/min; sample volume: 10 μL.

The target peak was eluted at 9.719 minutes and had the expected molecular weight, with a purity of 79.363%.

MS: m/z 1183.4

4.1 g of crude product was then dissolved in 50 mL of pure water and purified using Hanbon NP7010C semi-preparation equipment (Hanbon Sci. &Tech. Co., Ltd., Jiangsu, China). The preparation column model was a Dubhe-C18 model column (50*250 mm, 100A) (Hanbon Sci. & Tech. Co., Ltd., Jiangsu, China); detection: UV at 220 nm. The appropriate gradient for elution was calculated from LCMS detection step (Solvent A: 0.1% TFA in MeCN, solvent B: 0.1% TFA in water, with a linear gradient from 5%-20% solvent A concentration in 30 minutes; flow rate 60.0 mL/min;). Fractions were collected and analyzed using a Shimadzu LC-20 HPLC system (column as above, except with a linear gradient from 5%-30% solvent A concentration in 25 minutes) (Shimadzu Corporation, Kyoto, Japan).

Fractions with a purity of 98% were then mixed together for an anion exchange step. This was achieved using Hanbon NP7010C semi-preparation equipment, preparation column model: Dubhe-C18 model (as above). The fractions were diluted one time with pure water and loaded to the column directly, after that the column was washed with 3.2% of ammonium acetate in pure water for about 20 minutes followed by pure water for another 20 minutes at the flow rate of 60 mL/min, then eluted with the following gradient (Solvent A: 0.1% HAc in McCN, solvent B: 0.1% HAc in water, with a linear gradient from 5%-20% solvent A concentration in 30 minutes; flow rate 60.0 mL/min). Fractions were collected and analyzed using Shimadzu LC-20 HPLC system (column and conditions as above). Fractions with a purity of 98% were mixed and freeze-dried to give 2.38 g of the purified title compound.

### Example 8

Synthesis of Further Peptides I

The following peptides were synthesised using essentially the same procedure as that described in Example 7 above, with the exception that the appropriate amino acids were used in the relevant peptide coupling sequences:

(SEQ ID No: 30)
Ala-Lys-Pro-Ser-Tyr-Hyp-Thr-DOPA-Hyp-Lys,
(SEQ ID No: 115)
Lys-Pro-Ser-Tyr-Hyp-Hyp-Thr-DOPA-Hyp-Lys,
(SEQ ID No: 116)
Lys-Pro-Ser-Tyr-Hyp-Hyp-Thr-Tyr-Hyp-Lys,
(SEQ ID No: 117)
Lys-Pro-Ser-Tyr-Hyp-Thr-DOPA-Hyp-Lys,
(SEQ ID No: 118)
Lys-Pro-Ser-Tyr-Hyp-Thr-Tyr-Hyp-Lys,
(SEQ ID No: 119)
Ser-Lys-Pro-Ser-Tyr-Hyp-Hyp-Thr-DOPA-Lys,
(SEQ ID No: 120)
Ser-Lys-Pro-Ser-Tyr-Hyp-Hyp-Thr-Tyr-Lys,
(SEQ ID No: 121)
Ser-Lys-Pro-Ser-Tyr-Hyp-Thr-DOPA-Hyp-Lys,
(SEQ ID No: 122)
Ser-Lys-Pro-Ser-Tyr-Hyp-Thr-Tyr-Hyp-Lys,
(SEQ ID No: 123)
Ala-Lys-Pro-Ser-Tyr-Hyp-Hyp-Thr-DOPA-Hyp-Lys,
(SEQ ID NO: 124)
Ala-Lys-Pro-Ser-Tyr-Hyp-Hyp-Thr-Tyr-Hyp-Lys.

The crude yields and purity, retention time, MS values and final yields from these peptide syntheses are as shown in Table 1 below.

TABLE 1

| SEQ ID | Crude | Crude | Retention | | Final |
|---|---|---|---|---|---|
| No. | amount | purity | time | MS | amount |
| 30 | 4.09 g | 75.674% | 9.562 | 1199.3 | 2.46 g |
| 115 | 4.31 g | 76.035% | 9.873 | 1230.5 | 2.55 g |
| 116 | 4.27 g | 73.958% | 9.965 | 1214.2 | 2.47 g |
| 117 | 3.92 g | 78.932% | 9.214 | 1128.3 | 2.12 g |
| 118 | 3.89 g | 79.035% | 9.365 | 1112.3 | 2.09 g |
| 119 | 4.56 g | 76.933% | 9.847 | 1215.3 | 2.55 g |
| 120 | 4.49 g | 75.338% | 9.741 | 1199.3 | 2.52 g |
| 121 | 4.61 g | 77.018% | 9.798 | 1215.3 | 2.49 g |
| 122 | 4.52 g | 76.229% | 9.693 | 1199.3 | 2.47 g |
| 123 | 5.15 g | 73.943% | 9.383 | 1312.4 | 2.88 g |
| 124 | 5.07 g | 72.868% | 9.378 | 1296.4 | 2.79 g |

### Example 9

Chloramine-T Method for Radiolabeling Synthetic Peptides by Radioactive Iodine-¹²⁵I-Ala-Lys-Pro-Ser-Tyr-Hyp-Thr-Tyr-Hyp-Lys (incorporating SEQ ID No: 29)

Chloramine-T (p-toluene sulfonochloramine) is an effective method of labeling a variety of proteins and peptides. This oxidative method involves exposure of the substrate to Chloramine-T in the presence of NaI (in which different radioactive iodines are used, such as ¹²³I⁻, ¹²⁴I⁻, ¹²⁵I⁻, ¹²⁹I⁻ or ¹³¹I⁻) for a short time and produces high specific activity proteins or peptides labeled with carrier-free radioactive iodines, but can be harsh. The substitution of ¹²³I⁻, ¹²⁴I⁻, ¹²⁵I⁻, ¹²⁹I⁻ or ¹³¹I⁻ Into tyrosine residues in an oxido-reducing reaction is applicable to peptides and proteins naturally containing, or chemically modified to Introduce either tyrosine (or DOPA), arginine or histidine. Almost all the synthetic peptides have tyrosine or DOPA residues on them, so it is feasible to radiolabel synthetic peptide by ¹²³I⁻, ¹²⁴I⁻, ¹²⁵I⁻, ¹²⁹I⁻ or 131I⁻ using a Chloramine-T method.

5 μg of synthetic peptide (such as synthetic peptides SEQ ID No: 29) is firstly dissolved in 10 μL of pure water. 30 μL 0.5M phosphate buffer, pH7.4 is then added and mixed well. An amount (such as 74MBq/10 uL) of Na¹²⁵I solution is then added and mixed well. 100 μg of Chloramine-T in 10 μL of pure water is then added and mixed well quickly. The mixture is let to react under room temperature for about 1 to 3 minutes. The reaction is then stopped by adding 0.2 mL of Na₂S₂O₅ pure water solution, which contains Na₂S₂O₅ 200 μg. Finally, the mixture is desalted by Sephadex G50 column, and ¹²⁵I⁻ labelled synthetic peptide is prepared.

### Example 10

Synthesis of Further Peptides II

The following peptides are synthesised using essentially the same procedure as that described in Example 9 above, with the except that the appropriate peptide component is Incorporated with the appropriate radioactive iodine atoms:

(incorporating SEQ ID No: 30)
¹²⁵I-Ala-Lys-Pro-Ser-Tyr-Hyp-Thr-DOPA-Hyp-Lys,
(incorporating SEQ ID No: 115)
¹²⁵I-Lys-Pro-Ser-Tyr-Hyp-Hyp-Thr-DOPA-Hyp-Lys,
(incorporating SEQ ID No: 116)
¹²⁵I-Lys-Pro-Ser-Tyr-Hyp-Hyp-Thr-Tyr-Hyp-Lys,
(incorporating SEQ ID No: 117)
¹²⁵I-Lys-Pro-Ser-Tyr-Hyp-Thr-DOPA-Hyp-Lys,
(incorporating SEQ ID No: 118)
¹²⁵I-Lys-Pro-Ser-Tyr-Hyp-Thr-Tyr-Hyp-Lys,
(incorporating SEQ ID No: 119)
¹²⁵I-Ser-Lys-Pro-Ser-Tyr-Hyp-Hyp-Thr-DOPA-Lys,
(incorporating SEQ ID No: 120)
¹²⁵I-Ser-Lys-Pro-Ser-Tyr-Hyp-Hyp-Thr-Tyr-Lys,
(incorporating SEQ ID No: 121)
¹²⁵I-Ser-Lys-Pro-Ser-Tyr-Hyp-Thr-DOPA-Hyp-Lys,
(incorporating SEQ ID No: 122)
¹²⁵I-Ser-Lys-Pro-Ser-Tyr-Hyp-Thr-Tyr-Hyp-Lys,
(incorporating SEQ ID No: 123)
¹²⁵I-Ala-Lys-Pro-Ser-Tyr-Hyp-Hyp-Thr-DOPA-Hyp-Lys,
(incorporating SEQ ID No: 124)
¹²⁵I-Ala-Lys-Pro-Ser-Tyr-Hyp-Hyp-Thr-Tyr-Hyp-Lys,
(incorporating SEQ ID No: 30)
¹³¹I-Ala-Lys-Pro-Ser-Tyr-Hyp-Thr-DOPA-Hyp-Lys,
(incorporating SEQ ID No: 115)
¹³¹I-Lys-Pro-Ser-Tyr-Hyp-Hyp-Thr-DOPA-Hyp-Lys,
(incorporating SEQ ID No: 116)
¹³¹I-Lys-Pro-Ser-Tyr-Hyp-Hyp-Thr-Tyr-Hyp-Lys,
(incorporating SEQ ID No: 117)
¹³¹I-Lys-Pro-Ser-Tyr-Hyp-Thr-DOPA-Hyp-Lys,
(incorporating SEQ ID No: 118)
¹³¹I-Lys-Pro-Ser-Tyr-Hyp-Thr-Tyr-Hyp-Lys,
(incorporating SEQ ID No: 119)
¹³¹I-Ser-Lys-Pro-Ser-Tyr-Hyp-Hyp-Thr-DOPA-Lys,
(incorporating SEQ ID No: 120)
¹³¹I-Ser-Lys-Pro-Ser-Tyr-Hyp-Hyp-Thr-Tyr-Lys,
(incorporating SEQ ID No: 121)
¹³¹I-Ser-Lys-Pro-Ser-Tyr-Hyp-Thr-DOPA-Hyp-Lys,
(incorporating SEQ ID No: 122)
¹³¹I-Ser-Lys-Pro-Ser-Tyr-Hyp-Thr-Tyr-Hyp-Lys,
(incorporating SEQ ID No: 123)
¹³¹I-Ala-Lys-Pro-Ser-Tyr-Hyp-Hyp-Thr-DOPA-Hyp-Lys,
(incorporating SEQ ID No: 124)
¹³¹I-Ala-Lys-Pro-Ser-Tyr-Hyp-Hyp-Thr-Tyr-Hyp-Lys.

Example 11

(incorporating SEQ ID No: 29)
DOTA-Ala-Lys-Pro-Ser-Tyr-Hyp-Thr-Tyr-Hyp-Lys

The title compound was prepared using essentially the same process as described in Example 7 above, with the exception that after Fmoc-Ala-OH was coupled, one more compound DOTA-tris(tBu)ester (bifunctional chelating agents) was coupled in the same process as described in Example 7 above.

MS: m/z 1569.7

Repeating essentially the same procedure gave a further batch of crude title compound (yield 5.22 g). Analysis showed a target peak that was eluted at 8.012 minutes with the expected molecular weight (MS: m/z 1569.7). The purity was 73.812%.

5.2 g of the crude product was then purified as described in Example 7 above to give 3.0 g of pure title compound after freeze-drying.

### Example 12

Synthesis of Further Peptides III

The following peptides are synthesised using essentially the same procedure as that described in Example 7 above, with the exception that the appropriate amino acids are used in the relevant peptide coupling sequences:

(incorporating SEQ ID No: 30)
DOTA-Ala-Lys-Pro-Ser-Tyr-Hyp-Thr-DOPA-Hyp-Lys,
(incorporating SEQ ID No: 115)
DOTA-Lys-Pro-Ser-Tyr-Hyp-Hyp-Thr-DOPA-Hyp-Lys,
(incorporating SEQ ID No: 116)
DOTA-Lys-Pro-Ser-Tyr-Hyp-Hyp-Thr-Tyr-Hyp-Lys,
(incorporating SEQ ID No: 117)
DOTA-Lys-Pro-Ser-Tyr-Hyp-Thr-DOPA-Hyp-Lys,
(incorporating SEQ ID No: 118)
DOTA-Lys-Pro-Ser-Tyr-Hyp-Thr-Tyr-Hyp-Lys,
(incorporating SEQ ID No: 119)
DOTA-Ser-Lys-Pro-Ser-Tyr-Hyp-Hyp-Thr-DOPA-Lys,
(incorporating SEQ ID No: 120)
DOTA-Ser-Lys-Pro-Ser-Tyr-Hyp-Hyp-Thr-Tyr-Lys,
(incorporating SEQ ID No: 121)
DOTA-Ser-Lys-Pro-Ser-Tyr-Hyp-Thr-DOPA-Hyp-Lys,
(incorporating SEQ ID No: 122)
DOTA-Ser-Lys-Pro-Ser-Tyr-Hyp-Thr-Tyr-Hyp-Lys,
(incorporating SEQ ID No: 123)
DOTA-Ala-Lys-Pro-Ser-Tyr-Hyp-Hyp-Thr-DOPA-Hyp-Lys,
(incorporating SEQ ID No: 124)
DOTA-Ala-Lys-Pro-Ser-Tyr-Hyp-Hyp-Thr-Tyr-Hyp-Lys.

### Example 13

Synthesis of Further Peptides IV

The following peptides are synthesised using essentially the same procedure as that described in Example 11 above, with the exception that the appropriate amino acids are incorporated with the appropriate bifunctional chelating agents:

(incorporating SEQ ID No: 29)
NOTA-Ala-Lys-Pro-Ser-Tyr-Hyp-Thr-Tyr-Hyp-Lys,
(incorporating SEQ ID No: 29)
TETA-Ala-Lys-Pro-Ser-Tyr-Hyp-Thr-Tyr-Hyp-Lys,
(incorporating SEQ ID No: 29)
ATSM-Ala-Lys-Pro-Ser-Tyr-Hyp-Thr-Tyr-Hyp-Lys,
(incorporating SEQ ID No: 29)
PTSM-Ala-Lys-Pro-Ser-Tyr-Hyp-Thr-Tyr-Hyp-Lys,
(incorporating SEQ ID No: 29)
CB-TE2A-Ala-Lys-Pro-Ser-Tyr-Hyp-Thr-Tyr-Hyp-Lys,
(incorporating SEQ ID No: 164)
NOTA-Ala-Lys-Pro-Ser-Tyr-Hyp-Thr-Tyr-DOPA-Lys,
(incorporating SEQ ID No: 164)
TETA-Ala-Lys-Pro-Ser-Tyr-Hyp-Thr-Tyr-DOPA-Lys,
(incorporating SEQ ID No: 164)
ATSM-Ala-Lys-Pro-Ser-Tyr-Hyp-Thr-Tyr-DOPA-Lys,
(incorporating SEQ ID No: 164)
PTSM-Ala-Lys-Pro-Ser-Tyr-Hyp-Thr-Tyr-DOPA-Lys,
(incorporating SEQ ID No: 164)
CB-TE2A-Ala-Lys-Pro-Ser-Tyr-Hyp-Thr-Tyr-DOPA-Lys,
(incorporating SEQ ID No: 124)
NOTA-Ala-Lys-Pro-Ser-Tyr-Hyp-Hyp-Thr-Tyr-Hyp-Lys,
(incorporating SEQ ID No: 124)
TETA-Ala-Lys-Pro-Ser-Tyr-Hyp-Hyp-Thr-Tyr-Hyp-Lys,
(incorporating SEQ ID No: 124)
ATSM-Ala-Lys-Pro-Ser-Tyr-Hyp-Hyp-Thr-Tyr-Hyp-Lys,
(incorporating SEQ ID No: 124)
PTSM-Ala-Lys-Pro-Ser-Tyr-Hyp-Hyp-Thr-Tyr-Hyp-Lys,
(incorporating SEQ ID No: 124)
CB-TE2A-Ala-Lys-Pro-Ser-Tyr-Hyp-Hyp-Thr-Tyr-Hyp-
Lys.

Example 14

(incorporating SEQ ID No: 29)
⁶⁸Ga-DOTA-Ala-lys-Pro-Ser-Tyr-Hyp-Thr-Tyr-Hyp-Lys

The ⁶⁸Ga Generator is obtained from HTA Co. Ltd. (Beijing, China) with the activity of 1480 MBq (40 mCi). For full automation a PC-controlled radiopharmaceutical synthesis device PGP-74,DNA based on modular concept (HTA Co. Ltd., Beijing, China) is used for all steps in the synthesis. The ⁶⁸Ga Generator is eluted with 0.1M Ha.

A fraction of the generator eluate is added to one-tenth of the eluate volume of a solution containing 20 μg of DOTA-Ala-lys-Pro-Ser-Tyr-Hyp-Thr-Tyr-Hyp-Lys peptide in 1.25 mol/L sodium acetate solution, (pH 3.5). The solution is heated at 95° C. for 7 min (3 min preheating) and transferred to a preconditioned C-18 reversed phase cartridge (SEP PAK Mini Waters) for purification. The cartridge is eluted with ethanol over sterile filter (Millex-GV, Millipore) Into the final sterile vial. Subsequently, cartridge and sterile filter are washed with saline to dilute final ethanol content. The whole process from elution of the generator to the final product is performed within 12 min and the whole process is performed with fully automated PC-controlled radiopharmaceutical synthesis device based on a modular concept.

### Example 15

Synthesis of Further Peptides V

The following peptides are synthesised using essentially the same procedure as that described in Example 14 above, with the exception that the appropriate peptides are Incorporated with the relevant ⁶⁸Ga label:

(incorporating SEQ ID No: 30)
⁶⁸Ga-DOTA-Ala-Lys-Pro-Ser-Tyr-Hyp-Thr-DOPA-Hyp-Lys,
(incorporating SEQ ID No: 115)
⁶⁸Ga-DOTA-Lys-Pro-Ser-Tyr-Hyp-Hyp-Thr-DOPA-Hyp-Lys,
(incorporating SEQ ID No: 116)
⁶⁸Ga-DOTA-Lys-Pro-Ser-Tyr-Hyp-Hyp-Thr-Tyr-Hyp-Lys,
(incorporating SEQ ID No: 117)
⁶⁸Ga-DOTA-Lys-Pro-Ser-Tyr-Hyp-Thr-DOPA-Hyp-Lys,
(incorporating SEQ ID No: 118)
⁶⁸Ga-DOTA-Lys-Pro-Ser-Tyr-Hyp-Thr-Tyr-Hyp-Lys,
(incorporating SEQ ID No: 119)
⁶⁸Ga-DOTA-Ser-Lys-Pro-Ser-Tyr-Hyp-Hyp-Thr-DOPA-Lys,
(incorporating SEQ ID No: 120)
⁶⁸Ga-DOTA-Ser-Lys-Pro-Ser-Tyr-Hyp-Hyp-Thr-Tyr-Lys,
(incorporating SEQ ID No: 121)
⁶⁸Ga-DOTA-Ser-Lys-Pro-Ser-Tyr-Hyp-Thr-DOPA-Hyp-Lys,
(incorporating SEQ ID No: 122)
⁶⁸Ga-DOTA-Ser-Lys-Pro-Ser-Tyr-Hyp-Thr-Tyr-Hyp-Lys,
(incorporating SEQ ID No: 123)
⁶⁸Ga-DOTA-Ala-Lys-Pro-Ser-Tyr-Hyp-Hyp-Thr-DOPA-Hyp-Lys,
(incorporating SEQ ID No: 124)
⁶⁸Ga-DOTA-Ala-Lys-Pro-Ser-Tyr-Hyp-Hyp-Thr-Tyr-Hyp-Lys,
(incorporating SEQ ID No: 29)
⁶⁸Ga-NOTA-Ala-Lys-Pro-Ser-Tyr-Hyp-Thr-Tyr-Hyp-Lys,
(incorporating SEQ ID No: 29)
⁶⁸Ga-TETA-Ala-Lys-Pro-Ser-Tyr-Hyp-Thr-Tyr-Hyp-Lys,
(incorporating SEQ ID No: 29)
⁶⁸Ga-ATSM-Ala-Lys-Pro-Ser-Tyr-Hyp-Thr-Tyr-Hyp-Lys,
(incorporating SEQ ID No: 29)
⁶⁸Ga-PTSM-Ala-Lys-Pro-Ser-Tyr-Hyp-Thr-Tyr-Hyp-Lys,
(incorporating SEQ ID No: 29)
⁶⁸Ga-CB-TE2A-Ala-Lys-Pro-Ser-Tyr-Hyp-Thr-Tyr-Hyp-Lys,
(incorporating SEQ ID No: 164)
⁶⁸Ga-NOTA-Ala-Lys-Pro-Ser-Tyr-Hyp-Thr-Tyr-DOPA-Lys,
(incorporating SEQ ID No: 164)
⁶⁸Ga-TETA-Ala-Lys-Pro-Ser-Tyr-Hyp-Thr-Tyr-DOPA-Lys,
(incorporating SEQ ID No: 164)
⁶⁸Ga-ATSM-Ala-Lys-Pro-Ser-Tyr-Hyp-Thr-Tyr-DOPA-Lys,
(incorporating SEQ ID No: 164)
⁶⁸Ga-PTSM-Ala-Lys-Pro-Ser-Tyr-Hyp-Thr-Tyr-DOPA-Lys,
(incorporating SEQ ID No: 164)
⁶⁸Ga-CB-TE2A-Ala-Lys-Pro-Ser-Tyr-Hyp-Thr-Tyr-DOPA-Lys,
(incorporating SEQ ID No: 124)
⁶⁸Ga-NOTA-Ala-Lys-Pro-Ser-Tyr-Hyp-Hyp-Thr-Tyr-Hyp-Lys,
(incorporating SEQ ID No: 124)
⁶⁸Ga-TETA-Ala-Lys-Pro-Ser-Tyr-Hyp-Hyp-Thr-Tyr-Hyp-Lys,
(incorporating SEQ ID No: 124)
⁶⁸Ga-ATSM-Ala-Lys-Pro-Ser-Tyr-Hyp-Hyp-Thr-Tyr-Hyp-Lys,
(incorporating SEQ ID No: 124)
⁶⁸Ga-PTSM-Ala-Lys-Pro-Ser-Tyr-Hyp-Hyp-Thr-Tyr-Hyp-Lys,
(incorporating SEQ ID No: 124)
⁶⁸Ga-CB-TE2A-Ala-Lys-Pro-Ser-Tyr-Hyp-Hyp-Thr-Tyr-Hyp-Lys.

### Example 16

Synthesis of Further Peptides VI

Other radioactive atoms including ⁶⁴Cu, ⁹⁰Y, ¹¹¹In, ¹⁷Lu, ^{99m}Tc, ¹²⁵I, ¹⁸F, etc., are also used in the synthesis of different radioactive atom-labeled peptides. The method used is essentially the same procedure as that described in Example 14 above, with the except that the appropriate peptide sequence Is Incorporated with the appropriate radioactive atoms:

(incorporating SEQ ID No: 29)
⁶⁴Cu-DOTA-Ala-lys-Pro-Ser-Tyr-Hyp-Thr-Tyr-Hyp-Lys,
(incorporating SEQ ID No: 29)
⁹⁰Y-DOTA-Ala-lys-Pro-Ser-Tyr-Hyp-Thr-Tyr-Hyp-Lys,
(incorporating SEQ ID No: 29)
¹¹¹In-DOTA-Ala-lys-Pro-Ser-Tyr-Hyp-Thr-Tyr-Hyp-Lys,
(incorporating SEQ ID No: 29)
¹⁷⁷Lu-DOTA-Ala-lys-Pro-Ser-Tyr-Hyp-Thr-Tyr-Hyp-Lys,
(incorporating SEQ ID No: 29)
^{99m}Tc-DOTA-Ala-lys-Pro-Ser-Tyr-Hyp-Thr-Tyr-Hyp-Lys,
(incorporating SEQ ID No: 29)
¹²⁵I-DOTA-Ala-lys-Pro-Ser-Tyr-Hyp-Thr-Tyr-Hyp-Lys,
(incorporating SEQ ID No: 29)
¹⁸F-DOTA-Ala-lys-Pro-Ser-Tyr-Hyp-Thr-Tyr-Hyp-Lys,
(incorporating SEQ ID No: 30)
⁶⁴Cu-DOTA-Ala-lys-Pro-Ser-Tyr-Hyp-Thr-DOPA-Hyp-
Lys,
(incorporating SEQ ID No: 30)
⁹⁰Y-DOTA-Ala-lys-Pro-Ser-Tyr-Hyp-Thr-DOPA-Hyp-Lys,
(incorporating SEQ ID No: 30)
111In-DOTA-Ala-lys-Pro-Ser-Tyr-Hyp-Thr-DOPA-Hyp-
Lys,
(incorporating SEQ ID No: 30)
¹⁷⁷Lu-DOTA-Ala-lys-Pro-Ser-Tyr-Hyp-Thr-DOPA-Hyp-
Lys,
(incorporating SEQ ID No: 30)
^{99m}Tc-DOTA-Ala-lys-Pro-Ser-Tyr-Hyp-Thr-DOPA-Hyp-
Lys,
(incorporating SEQ ID No: 30)
¹²5I-DOTA-Ala-lys-Pro-Ser-Tyr-Hyp-Thr-DOPA-Hyp-
Lys,
(incorporating SEQ ID No: 30)
¹⁸F-DOTA-Ala-lys-Pro-Ser-Tyr-Hyp-Thr-DOPA-Hyp-Lys.

1. A modified peptide compound comprising one or more of the peptide components (a),
(b) or (c) as defined below:
(a) a peptide component of formula I,
A-Q-B I
wherein:
A and B Independently represent Z or A¹-Q¹-B¹;
Q represents a structural fragment of formula II, 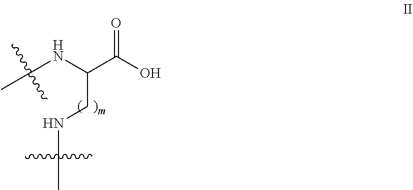
wherein:
the squiggly lines represent points of attachment of Q to A and/or B; and
m represents an Integer 1 to 4;
A¹ and B¹ Independently represent Z or A²-Q²-B²;
A² and B² Independently represent Z or Z-Q³-Z;
Q¹, Q² and Q³ Independently represent structural fragments of formula III, 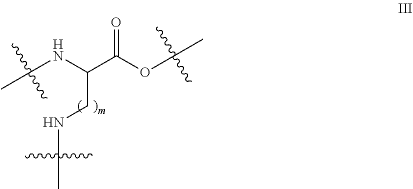
wherein:
the squiggly lines adjacent to the NH groups represent the points of attachment of Q¹, Q² and Q³ to A¹ and/or B¹, A² and/or B², and Z, respectively; and the squiggly line adjacent to the 0 atom represents the point of attachment of Q¹, Q² and Q³ to Q, Q¹ and Q², respectively; and m is as defined above;
on each occasion that it is employed, Z represents a peptide component of the amino acid sequence:
(SEQ ID NO: 3)
[W-Lys-X¹-Ser-U-X²-Y]ₙ-W-Lys-X¹-Ser-U-X²-Y---
wherein:
the dashed line represents the point of attachment of Z to the rest of the molecule;
n represents 0 or an Integer 1 to 4; and,
on each occasion that they are employed:
W represents a 1 or 2 amino acid sequence, in which the amino acids are selected from
one or more of the group Lys, Ala, DOPA and a 3,4-dihydrocinnamic add (HCA) residue, provided that, when present, the HCA residue is located at the N-terminus of the peptide sequence Z;
X¹ represents Pro, Hyp or diHyp;
U represents Tyr or DOPA;
X² represents Ser, Pro, Hyp or diHyp; and
Y represents a 1 to 5 amino acid sequence, in which the amino acids are selected from one or more of the group Lys, Ala, Pro, Hyp, diHyp, Thr, DOPA and Tyr; or
(b) a peptide component of the amino acid sequence:
(SEQ ID NO: 4)
[Ala-Lys-X¹-Ser-U-X²-Y]ₚ-Ala-Lys-X¹-Ser-U-X²-Y-G
wherein
p represents an Integer 1 to 4;
G may be absent or represents DOPA or dopamine; and
X¹, U, X² and Y are as defined above; or
(c) a peptide component of the amino acid sequence:
(SEQ ID NO: 5)
W¹-Lys-X¹-Ser-U¹-X²-Y-G
wherein:
W¹ is absent or represents a 1, 2 or 3 amino acid sequence, in which the amino acids are selected from one or more of the group Ser, Lys, Ala, DOPA and a 3,4-dihydrocinnamic acid (HCA) residue, provided that, when present, the HCA residue is located at the N-terminus of the peptide sequence;
U¹ represents Tyr, DOPA or a single bond; and
X¹, X², Y and G are as defined above,
wherein said one or more peptide components are modified to include a labelling group or component, which labelling group or component is capable of medically imaging areas of the human or animal body, capable of medically imaging human or animal body parts, and/or capable of diagnosing and/or therapeutically treating one or more cancers, in a patient, as well as regioisomers, stereoisomers, and pharmaceutically-acceptable salts of said compounds.

2. A compound as claimed in claim 1, wherein the labelling group comprises a molecule that is capable of non-invasively imaging parts of the body including vasculature networks in internal organs, cancer cells and/or tumours, by virtue of bioluminescence.

3. A compound as claimed in claim 2, wherein the labelling group is a fluorescein.

4. A compound as claimed in claim 1, wherein the labelling group is, or comprises, a radionuclide.

5. A compound as claimed in claim 5, wherein the radionuclide is selected from ³H, ¹¹C, ¹³C, ¹⁴C, ¹³N, ¹⁵O, ¹⁸F, ²²Na, ²⁴Na, ³²P, ³³P, ⁴²K, ⁴⁷Ca, ⁴⁷Sc, ⁵¹Cr, ⁵⁷Co, ⁵⁸Co, ⁵⁹Fe, ⁶⁰Co, ⁶⁴Cu, ⁶⁷Cu, ⁶⁷Ga, ⁶⁸Ga, ⁷⁵Se, ⁷⁷As, ^{80m}gr, ^{81m}Kr, ⁸²Rb, ⁸⁹Sr, ⁸⁹Zr, ⁹⁰Y, ⁹⁰Sr, ⁹⁹Mo, ^{99m}Tc, ¹⁰³Pd, ^{103m}Rh, ¹⁰⁵Rh, ¹⁰⁹Pd, ¹⁰⁹Pt, ¹¹¹Ag, ¹¹¹In, ¹¹⁹Sb, ¹²¹Sn, ¹²⁷Te, ¹²³I, ¹²⁵I, ¹²⁹I, ¹³¹I, ¹³³Xe, ¹⁴²pr, ¹⁴³Pr, ¹⁴⁹Pm, ¹⁵¹Pm, ¹⁵²Dy, ¹⁵³Sm, ¹⁵⁹Gd, ¹⁶¹Tb, ¹⁶¹Ho, ¹⁶⁵Dy, ¹⁶⁶Ho, ¹⁶⁶Dy, ¹⁶⁹Er, ¹⁶⁹Yb, ¹⁷⁵Yb, ¹⁷²Tm, ¹⁷⁷Lu, ^{177m}Sn, ¹⁸⁶Re, ¹⁸⁸Re, ¹⁸⁹Re, ¹⁸⁸Rd, ^{189m}Os, ¹⁹²Ir, ¹⁹⁴Ir, ¹⁹⁸Au, ¹⁹⁹Au, ²⁰¹Ti, ²¹¹At, ²¹¹Pb, ²¹²Pb, ²¹¹Bi, ²¹²Bi, ²¹³Bi, ²¹⁵Po, ²¹⁷At, ²¹⁹Rn, ²²¹Fr, ²²³Ra, ²²⁵Ac, ²²⁷Th, ²⁵⁵Fm and ¹²⁴I.

6. A compound as claimed in claim 4 or claim 5, wherein the radionuclide is attached directly to the peptide component.

7. A compound as claimed in claim 4 or claim 5, wherein the labelling component comprises a molecule that is capable of forming a complex with the radionuclide.

8. A compound as claimed in any one of claims 2 to 5 or 7, wherein the labelling group or the other molecule is attached to the peptide component via a linker moiety.

9. A compound as claimed in any one of the preceding claims, wherein, in the peptide component, W and/or W¹ represents HCA, HCA-Ala-, Ala, Lys-Ala, DOPA or DOPA-Ala-.

10. A compound as claimed in any one of the preceding claims, wherein, in the peptide component, X¹ represents Hyp or Pro.

11. A compound as claimed in any one of the preceding claims, wherein, in the peptide component, X² represents Ser, Pro or Hyp.

12. A compound as claimed in any one of the preceding claims, wherein, in the peptide component, Y represents a 3, a 4 or a 5 amino acid sequence, in which the amino acids are selected from one or more of the group Lys, Ala, Hyp, Thr, DOPA and Tyr.

13. A compound as claimed in any one of the preceding claims, wherein, in the case of peptide component (c), Y represents -Hyp-Thr-, -Thr-Tyr-, -Pro-Thr-, -Thr-DOPA-, -Thr-Tyr-Lys-, -Tyr-Pro-Lys-, -DOPA-Pro-Lys-, -Hyp-Thr-Tyr-, -Hyp-Thr-DOPA-, -Pro-Thr-DOPA-Lys-, -Pro-Thr-Tyr-Lys-, -Thr-Tyr-Pro-Lys-, -Thr-DOPA-Pro-Lys-, -Hyp-Thr-Tyr-Lys-, -Hyp-Thr-DOPA-Lys-, -Hyp-Thr-Ala-Lys-, -Thr-Tyr-Hyp-Lys-, -Thr-DOPA-Hyp-Lys-, -Thr-Ala-Hyp-Lys-, -Hyp-Thr-Tyr-Hyp-Lys-, -Thr-Tyr-Hyp-Lys-DOPA-, or -Thr-Tyr-DOPA-Lys-.

14. A compound as claimed in any one of the preceding claims, wherein the peptide component (c), is of the amino acid sequence:
(SEQ ID NO: 114)
K-W²-Lys-X¹-Ser-U¹-X²-Y¹-I-J
wherein K represents an optional N-terminal HCA group;
W² may be absent or represent a 1 or 2 amino acid sequence, in which the amino adds are selected from one or more of the group Ser, Lys, Ala and DOPA;
Y¹ represents a single bond or a 1 to 3 amino acid sequence, in which the amino acids are selected from one or more of the group Lys, Ala, Pro, Hyp, diHyp, Thr, DOPA and Tyr;
I represents Pro, Hyp, diHyp, Thr, DOPA or Tyr;
3 represents Lys or is absent; and
X¹, U¹ and X² are as defined in the relevant preceding claims.

15. A compound as claimed in claim 14, wherein W² represents Ala or Ser or is absent.

16. A compound as claimed in claim 15, wherein W² represents Ala.

17. A compound as claimed in any one of claims 14 to 16, wherein X² represents Pro, Hyp or diHyp.

18. A compound as claimed in claim 11 or claim 17, wherein X² represents Hyp.

19. A compound as claimed in any one of claims 14 to 18, wherein, when K is not present, W² represents Ala or is absent and 3 represents Lys, then I represents Pro, Hyp, diHyp or Thr.

20. A compound as claimed in any one of claims 14 to 19, wherein U¹ represents DOPA or Tyr.

21. A compound as claimed in any one of claims 14 to 20, wherein 3 represents Lys.

22. A compound as claimed in any one of claims 14 to 20, wherein 3 is absent.

23. A compound as claimed in any one of claims 14 to 22, wherein I represents DOPA, Tyr, Pro or Hyp.

24. A compound as claimed in any one of claims 14 to 23, wherein Y¹ represents a 1, a 2 or a 3 amino add sequence, in which the amino acids are selected from the group Pro, Hyp, Thr, DOPA and Tyr.

25. A compound as claimed in claim 24, in which, in the sequence defined by Y¹:
the amino add DOPA, Thr, Lys or Tyr is linked to I; and/or
the amino acid Pro, Hyp or Thr is linked to X².

26. A compound as claimed in claim 24 or claim 26, wherein Y¹ represents -Hyp-Thr-Tyr-, -Hyp-Thr-DOPA-, -Thr-DOPA-Lys-, -Thr-Tyr-Lys-, -Thr-Tyr-, -Thr-DOPA-, -Pro-Thr- or -Hyp-Thr-.

27. A compound as claimed in any one of claims 14 to 26, wherein K is absent.

28. A compound as claimed in claim 27, wherein W² is absent, Y¹ represents a single bond and 3 represents Lys.

29. A compound as claimed in claim 28, wherein:
X¹ represents Pro;
U¹ and I independently represent DOPA or Tyr; and/or
X² represents Hyp.

30. A compound as claimed in any one of the preceding claims, wherein the peptide component is a peptide component as defined by (c), and is selected from the group:
(SEQ ID No: 19)
Ala-Lys-Pro-Ser-Tyr-Hyp-Thr-Tyr-Hyp-Lys-DOPA;
(SEQ ID No: 20)
Ala-Lys-Pro-Ser-Tyr-Hyp-Thr-Tyr-Hyp-Lys-Dopamine;
(SEQ ID No: 21)
Ala-Lys-Pro-Ser-Tyr-Hyp-Hyp-Thr-Tyr-Lys-Dopamine;
(SEQ ID No: 22)
Ala-Lys-Pro-Ser-Tyr-Pro-Pro-Thr-DOPA-Lys;
(SEQ ID No: 23)
Ala-Lys-Pro-Ser-Tyr-Pro-Thr-Tyr-Pro-Lys;
(SEQ ID No: 24)
Ala-Lys-Pro-Ser-Tyr-Pro-Thr-DOPA-Pro-Lys;
(SEQ ID No: 25)
Ala-Lys-Pro-Ser-Tyr-Pro-Hyp-Thr-Tyr-Lys;
(SEQ ID No: 26)
Ala-Lys-Pro-Ser-Tyr-Pro-Hyp-Thr-DOPA-Lys;
(SEQ ID No: 27)
Ala-Lys-Pro-Ser-Tyr-Hyp-Pro-Thr-Tyr-Lys;
(SEQ ID No: 28)
Ala-Lys-Pro-Ser-Tyr-Hyp-Pro-Thr-DOPA-Lys;
(SEQ ID No: 29)
Ala-Lys-Pro-Ser-Tyr-Hyp-Thr-Tyr-Hyp-Lys;
(SEQ ID No: 30)
Ala-Lys-Pro-Ser-Tyr-Hyp-Thr-DOPA-Hyp-Lys;
(SEQ ID No: 31)
Ala-Lys-Pro-Ser-Tyr-Hyp-Hyp-Thr-Tyr-Lys-DOPA;
(SEQ ID No: 32)
Ala-Lys-Pro-Ser-Tyr-Hyp-Hyp-Thr-DOPA-Lys-DOPA;
(SEQ ID No: 33)
Ala-Lys-Pro-Ser-Tyr-Hyp-Hyp-Thr-DOPA-Lys-Dopamine;
(SEQ ID No: 34)
Ala-Lys-Pro-Ser-Tyr-Hyp-Thr-DOPA-Hyp-Lys-DOPA;
(SEQ ID No: 35)
Ala-Lys-Pro-Ser-Tyr-Hyp-Thr-DOPA-Hyp-Lys-Dopamine;
(SEQ ID No: 39)
Lys-Ala-Lys-Pro-Ser-Tyr-Hyp-Hyp-Thr-DOPA;
(SEQ ID No: 40)
Lys-Ala-Lys-Pro-Ser-Tyr-Hyp-Hyp-Thr-Dopamine;
(SEQ ID No: 41)
Lys-Ala-Lys-Hyp-Ser-Tyr-Hyp-Hyp-Thr-DOPA;
(SEQ ID No: 42)
Lys-Ala-Lys-Hyp-Ser-Tyr-Hyp-Hyp-Thr-Tyr;
(SEQ ID No: 43)
Lys-Ala-Lys-Pro-Ser-Tyr-Hyp-Hyp-Thr-Tyr.
(SEQ ID No: 52)
DOPA-Lys-Pro-Ser-Tyr-Hyp-Thr-Ala-Hyp-Lys;
(SEQ ID No: 53)
DOPA-Lys-Pro-Ser-Tyr-Hyp-Hyp-Thr-Ala-Lys;
(SEQ ID No: 54)
DOPA-Ala-Lys-Pro-Ser-Tyr-Hyp-Thr-Tyr-Hyp-Lys;
(SEQ ID No: 55)
DOPA-Ala-Lys-Pro-Ser-Tyr-Hyp-Hyp-Thr-Tyr-Lys;
(SEQ ID No: 56)
DOPA-Ala-Lys-Pro-Ser-Tyr-Hyp-Thr-DOPA-Hyp-Lys;
(SEQ ID No: 57)
DOPA-Ala-Lys-Pro-Ser-Tyr-Hyp-Hyp-Thr-DOPA-Lys;
(SEQ ID No: 58)
HCA-Lys-Pro-Ser-Tyr-Hyp-Hyp-Thr-Ala-Lys;
(SEQ ID No: 59)
HCA-Ala-Lys-Pro-Ser-Tyr-Hyp-Thr-Tyr-Hyp-Lys;
(SEQ ID No: 60)
HCA-Ala-Lys-Pro-Ser-Tyr-Hyp-Thr-DOPA-Hyp-Lys;
(SEQ ID No: 61)
HCA-Ala-Lys-Pro-Ser-Tyr-Hyp-Hyp-Thr-Tyr-Lys;
(SEQ ID No: 62)
HCA-Ala-Lys-Pro-Ser-Tyr-Hyp-Hyp-Thr-DOPA-Lys;
(SEQ ID No: 63)
HCA-Lys-Pro-Ser-Tyr-Hyp-Thr-Ala-Hyp-Lys.
(SEQ ID No: 70)
Lys-Ala-Lys-Pro-Ser-DOPA-Hyp-Hyp-Thr-Dopamine;
(SEQ ID No: 71)
Lys-Ala-Lys-Hyp-Ser-DOPA-Hyp-Hyp-Thr-DOPA;
(SEQ ID No: 72)
Lys-Ala-Lys-Pro-Ser-DOPA-Hyp-Hyp-Thr-DOPA;
(SEQ ID No: 73)
Ala-Lys-Pro-Ser-DOPA-Pro-Pro-Thr-Tyr-Lys;
(SEQ ID No: 74)
Ala-Lys-Pro-Ser-DOPA-Pro-Pro-Thr-DOPA-Lys;
(SEQ ID No: 75)
Ala-Lys-Pro-Ser-DOPA-Pro-Thr-Tyr-Pro-Lys;
(SEQ ID No: 76)
Ala-Lys-Pro-Ser-DOPA-Pro-Thr-DOPA-Pro-Lys;
(SEQ ID No: 77)
Ala-Lys-Pro-Ser-DOPA-Pro-Hyp-Thr-Tyr-Lys;
(SEQ ID No: 78)
Ala-Lys-Pro-Ser-DOPA-Pro-Hyp-Thr-DOPA-Lys;
(SEQ ID No: 79)
Ala-Lys-Pro-Ser-DOPA-Hyp-Pro-Thr-Tyr-Lys;
(SEQ ID No: 80)
Ala-Lys-Pro-Ser-DOPA-Hyp-Pro-Thr-DOPA-Lys;
(SEQ ID No: 81)
Ala-Lys-Pro-Ser-DOPA-Hyp-Thr-Tyr-Hyp-Lys;
(SEQ ID No: 82)
Ala-Lys-Pro-Ser-DOPA-Hyp-Thr-DOPA-Hyp-Lys;
(SEQ ID No: 83)
Lys-Ala-Lys-Pro-Ser-DOPA-Hyp-Hyp-Thr-Tyr;
(SEQ ID No: 84)
Lys-Ala-Lys-Hyp-Ser-DOPA-Hyp-Hyp-Thr-Tyr;
(SEQ ID No: 85)
Ala-Lys-Pro-Ser-DOPA-Hyp-Hyp-Thr-Tyr-Lys-DOPA;
(SEQ ID No: 86)
Ala-Lys-Pro-Ser-DOPA-Hyp-Hyp-Thr-DOPA-Lys-DOPA;
(SEQ ID No: 87)
Ala-Lys-Pro-Ser-DOPA-Hyp-Hyp-Thr-Tyr-Lys-Dopamine;
(SEQ ID No: 88)
Ala-Lys-Pro-Ser-DOPA-Hyp-Hyp-Thr-DOPA-Lys-Dopamine;
(SEQ ID No: 89)
Ala-Lys-Pro-Ser-DOPA-Hyp-Thr-Tyr-Hyp-Lys-DOPA;
(SEQ ID No: 90)
Ala-Lys-Pro-Ser-DOPA-Hyp-Thr-DOPA-Hyp-Lys-DOPA;
(SEQ ID No: 91)
Ala-Lys-Pro-Ser-DOPA-Hyp-Thr-Tyr-Hyp-Lys-Dopamine;
(SEQ ID No: 92)
Ala-Lys-Pro-Ser-DOPA-Hyp-Thr-DOPA-Hyp-Lys-Dopamine.
(SEQ ID No: 102)
DOPA-Lys-Pro-Ser-DOPA-Hyp-Thr-Ala-Hyp-Lys;
(SEQ ID No: 103)
DOPA-Lys-Pro-Ser-DOPA-Hyp-Hyp-Thr-Ala-Lys;
(SEQ ID No: 104)
DOPA-Ala-Lys-Pro-Ser-DOPA-Hyp-Thr-DOPA-Hyp-Lys;
(SEQ ID No: 105)
DOPA-Ala-Lys-Pro-Ser-DOPA-Hyp-Hyp-Thr-Tyr-Lys;
(SEQ ID No: 106)
DOPA-Ala-Lys-Pro-Ser-DOPA-Hyp-Hyp-Thr-DOPA-Lys;
(SEQ ID No: 107)
HCA-Ala-Lys-Pro-Ser-DOPA-Hyp-Thr-DOPA-Hyp-Lys;
(SEQ ID No: 108)
HCA-Ala-Lys-Pro-Ser-DOPA-Hyp-Hyp-Thr-DOPA-Lys;
(SEQ ID No: 109)
HCA-Lys-Pro-Ser-DOPA-Hyp-Thr-Ala-Hyp-Lys;
(SEQ ID No: 110)
HCA-Lys-Pro-Ser-DOPA-Hyp-Hyp-Thr-Ala-Lys;
(SEQ ID No: 111)
DOPA-Ala-Lys-Pro-Ser-DOPA-Hyp-Thr-Tyr-Hyp-Lys;
(SEQ ID No: 112)
HCA-Ala-Lys-Pro-Ser-DOPA-Hyp-Thr-Tyr-Hyp-Lys;
(SEQ ID No: 113)
HCA-Ala-Lys-Pro-Ser-DOPA-Hyp-Hyp-Thr-Tyr-Lys;
(SEQ ID No: 115)
Lys-Pro-Ser-Tyr-Hyp-Hyp-Thr-DOPA-Hyp-Lys;
(SEQ ID No: 116)
Lys-Pro-Ser-Tyr-Hyp-Hyp-Thr-Tyr-Hyp-Lys;
(SEQ ID No: 117)
Lys-Pro-Ser-Tyr-Hyp-Thr-DOPA-Hyp-Lys;
(SEQ ID No: 118)
Lys-Pro-Ser-Tyr-Hyp-Thr-Tyr-Hyp-Lys;
(SEQ ID No: 119)
Ser-Lys-Pro-Ser-Tyr-Hyp-Hyp-Thr-DOPA-Lys;
(SEQ ID No: 120)
Ser-Lys-Pro-Ser-Tyr-Hyp-Hyp-Thr-Tyr-Lys;
(SEQ ID No: 121)
Ser-Lys-Pro-Ser-Tyr-Hyp-Thr-DOPA-Hyp-Lys;
(SEQ ID No: 122)
Ser-Lys-Pro-Ser-Tyr-Hyp-Thr-Tyr-Hyp-Lys;
(SEQ ID No: 123)
Ala-Lys-Pro-Ser-Tyr-Hyp-Hyp-Thr-DOPA-Hyp-Lys;
(SEQ ID No: 124)
Ala-Lys-Pro-Ser-Tyr-Hyp-Hyp-Thr-Tyr-Hyp-Lys;
(SEQ ID No: 125)
Ala-Lys-Pro-Ser-Tyr-Hyp-Thr-DOPA-Hyp-Lys;
(SEQ ID No: 126)
Ala-Lys-Pro-Ser-Tyr-Hyp-Thr-Tyr-Hyp-Lys;
(SEQ ID No: 127)
Lys-Pro-Ser-Tyr-Hyp-Hyp-Thr-DOPA-Hyp;
(SEQ ID No: 128)
Lys-Pro-Ser-Tyr-Hyp-Hyp-Thr-Tyr-Hyp;
(SEQ ID No: 129)
Lys-Pro-Ser-Tyr-Hyp-Thr-DOPA-Hyp;
(SEQ ID No: 130)
Lys-Pro-Ser-Tyr-Hyp-Thr-Tyr-Hyp;
(SEQ ID No: 131)
Ser-Lys-Pro-Ser-Tyr-Hyp-Hyp-Thr-Tyr;
(SEQ ID No: 132)
Ser-Lys-Pro-Ser-Tyr-Hyp-Thr-DOPA-Hyp;
(SEQ ID No: 133)
Ser-Lys-Pro-Ser-Tyr-Hyp-Thr-Tyr-Hyp;
(SEQ ID No: 134)
Ala-Lys-Pro-Ser-Tyr-Hyp-Hyp-Thr-DOPA-Hyp;
(SEQ ID No: 135)
Ala-Lys-Pro-Ser-Tyr-Hyp-Hyp-Thr-Tyr-Hyp;
(SEQ ID No: 136)
Ala-Lys-Pro-Ser-Tyr-Hyp-Thr-DOPA-Hyp;
(SEQ ID No: 137)
Ala-Lys-Pro-Ser-Tyr-Hyp-Thr-Tyr-Hyp;
(SEQ ID No: 138)
Ser-Lys-Pro-Ser-Tyr-Hyp-Hyp-Thr-DOPA;
(SEQ ID No: 139)
HCA-Ala-Lys-Pro-Ser-Tyr-Hyp-Hyp-Thr-Tyr-Lys;
(SEQ ID No: 140)
HCA-Ala-Lys-Pro-Ser-Tyr-Hyp-Thr-Tyr-Hyp-Lys;
(SEQ ID No: 141)
Ala-Lys-Pro-Ser-DOPA-Hyp-Thr-DOPA-Hyp-Lys;
(SEQ ID No: 142)
Ala-Lys-Pro-Ser-Pro-Thr-Tyr-Pro-Lys;
(SEQ ID No: 143)
Ala-Lys-Hyp-Ser-Tyr-Hyp-Thr-Tyr-Hyp-Lys;
(SEQ ID No: 144)
Ala-Lys-Hyp-Ser-DOPA-Hyp-Thr-DOPA-Hyp-Lys;
(SEQ ID No: 145)
Ala-Lys-Hyp-Ser-Tyr-Hyp-Thr-DOPA-Hyp-Lys;
(SEQ ID No: 146)
HCA-Ala-Lys-Pro-Ser-Tyr-Hyp-Hyp-Thr-Tyr;
(SEQ ID No: 147)
HCA-Ala-Lys-Pro-Ser-Tyr-Hyp-Thr-Tyr-Hyp;
(SEQ ID No: 148)
Ala-Lys-Pro-Ser-DOPA-Hyp-Thr-DOPA-Hyp;
(SEQ ID No: 149)
Ala-Lys-Pro-Ser-Pro-Thr-Tyr-Pro;
(SEQ ID No: 150)
Ala-Lys-Hyp-Ser-Tyr-Hyp-Thr-Tyr-Hyp;
(SEQ ID No: 151)
Ala-Lys-Hyp-Ser-DOPA-Hyp-Thr-DOPA-Hyp;
(SEQ ID No: 152)
Ala-Lys-Hyp-Ser-Tyr-Hyp-Thr-DOPA-Hyp;
(SEQ ID No: 153)
Ala-Lys-Pro-Ser-DOPA-Hyp-Thr-Tyr-Lys-Hyp;
(SEQ ID No: 154)
Ala-Lys-Hyp-Ser-DOPA-Hyp-Thr-Tyr-Lys-Hyp;
(SEQ ID No: 155)
Ala-Lys-Pro-Ser-Tyr-Hyp-Thr-Tyr-Lys-Hyp;
(SEQ ID No: 156)
Ala-Lys-Hyp-Ser-Tyr-Hyp-Thr-Tyr-Lys-Hyp;
(SEQ ID No: 157)
Ala-Lys-Pro-Ser-Tyr-Hyp-Thr-DOPA-Lys-Hyp;
(SEQ ID No: 158)
Ala-Lys-Hyp-Ser-Tyr-Hyp-Thr-DOPA-Lys-Hyp;
(SEQ ID No: 159)
Ala-Lys-Pro-Ser-DOPA-Hyp-Thr-DOPA-Lys-Hyp;
(SEQ ID No: 160)
Ala-Lys-Hyp-Ser-DOPA-Hyp-Thr-DOPA-Lys-Hyp;
(SEQ ID No: 162)
Lys-Pro-Ser-Tyr-Hyp-DOPA-Lys;
or
(SEQ ID No: 163)
Lys-Pro-Ser-Tyr-Hyp-Tyr-Lys.

31. A compound as claimed in any one of the preceding claims, wherein the peptide component is selected from the group:
(SEQ ID No: 1)
Ala-Lys-Pro-Ser-Tyr-Hyp-Hyp-Thr-DOPA-Lys;
(SEQ ID No: 2)
Ala-Lys-Pro-Ser-Tyr-Hyp-Hyp-Thr-Tyr-Lys;
(SEQ ID No: 12)
Ala-Lys-Pro-Ser-Tyr-Hyp-Thr-Tyr-Hyp-Lys;
(SEQ ID No: 155)
Ala-Lys-Pro-Ser-Tyr-Hyp-Thr-Tyr-Lys-Hyp;
(SEQ ID No: 162)
Lys-Pro-Ser-Tyr-Hyp-DOPA-Lys;
or
(SEQ ID NO: 163)
Lys-Pro-Ser-Tyr-Hyp-Tyr-Lys.

32. A compound as claimed in claims 1 to 30, wherein the peptide component is selected from the group:
(SEQ ID No: 29)
Ala-Lys-Pro-Ser-Tyr-Hyp-Thr-Tyr-Hyp-Lys;
(SEQ ID No: 30)
Ala-Lys-Pro-Ser-Tyr-Hyp-Thr-DOPA-Hyp-Lys;
(SEQ ID No: 115)
Lys-Pro-Ser-Tyr-Hyp-Hyp-Thr-DOPA-Hyp-Lys;
(SEQ ID No: 116)
Lys-Pro-Ser-Tyr-Hyp-Hyp-Thr-Tyr-Hyp-Lys;
(SEQ ID No: 117)
Lys-Pro-Ser-Tyr-Hyp-Thr-DOPA-Hyp-Lys;
(SEQ ID No: 118)
Lys-Pro-Ser-Tyr-Hyp-Thr-Tyr-Hyp-Lys;
(SEQ ID No: 119)
Ser-Lys-Pro-Ser-Tyr-Hyp-Hyp-Thr-DOPA-Lys;
(SEQ ID No: 120)
Ser-Lys-Pro-Ser-Tyr-Hyp-Hyp-Thr-Tyr-Lys;
(SEQ ID No: 121)
Ser-Lys-Pro-Ser-Tyr-Hyp-Thr-DOPA-Hyp-Lys;
(SEQ ID No: 122)
Ser-Lys-Pro-Ser-Tyr-Hyp-Thr-Tyr-Hyp-Lys;
(SEQ ID No: 123)
Ala-Lys-Pro-Ser-Tyr-Hyp-Hyp-Thr-DOPA-Hyp-Lys;
(SEQ ID No: 124)
Ala-Lys-Pro-Ser-Tyr-Hyp-Hyp-Thr-Tyr-Hyp-Lys;
(SEQ ID NO. 164)
Ala-Lys-Pro-Ser-Tyr-Hyp-Thr-Tyr-DOPA-Lys

33. A compound as claimed in any one of claims 1 to 12, wherein, when the peptide component is defined by (a), m represents 4, such that one or more of Q, Q¹, Q² and Q³ represent a Lys fragment.

34. A compound as claimed in claim 30, wherein A and B both represent Z or A¹-Q¹-B¹, A¹ and B¹ both represent Z or A²-Q²-B², and/or A² and B² both represent Z.

35. A compound as claimed in claim 33 or claim 34, wherein Z represents:
(SEQ ID No: 2)
Ala-Lys-Pro-Ser-Tyr-Hyp-Hyp-Thr-Tyr-Lys---;
(SEQ ID No: 1)
Ala-Lys-Pro-Ser-Tyr-Hyp-Hyp-Thr-DOPA-Lys---;
(SEQ ID No: 14)
Ala-Lys-Pro-Ser-Tyr-Hyp-Thr-Tyr-Hyp-Lys---;
(SEQ ID No: 15)
Ala-Lys-Pro-Ser-Tyr-Hyp-Thr-DOPA-Hyp-Lys---;
(SEQ ID No: 16)
Ala-Lys-Pro-Ser-Tyr-Hyp-Thr-Tyr-Hyp-Lys-DOPA---;
(SEQ ID No: 17)
Ala-Lys-Pro-Ser-Tyr-Ser-Hyp-Thr-Tyr-Lys-Ala-Lys-Pro-Ser-Tyr-Ser-Hyp-Thr-Tyr-Lys--;
(SEQ ID No: 18)
Ala-Lys-Pro-Ser-Tyr-Hyp-Hyp-Thr-Tyr-Lys-Ala-Lys-Pro-Ser-Tyr-Hyp-Hyp-Thr-Tyr-Lys-Ala-
Lys-Pro-Ser-Tyr-Hyp-Hyp-Thr-Tyr-Lys-Ala-Lys-Pro-Ser-Tyr-Hyp-Hyp-Thr-Tyr-Lys-Ala-
Lys-Pro-Ser-Tyr-Hyp-Hyp-Thr-Tyr-Lys---;
(SEQ ID No: 36)
Lys-Ala-Lys-Pro-Ser-Tyr-Hyp-Hyp-Thr-Tyr---;
(SEQ ID No: 37)
Lys-Ala-Lys-Hyp-Ser-Tyr-Hyp-Hyp-Thr-DOPA---;
(SEQ ID No: 38)
Lys-Ala-Lys-Pro-Ser-Tyr-Hyp-Hyp-Thr-DOPA---;
(SEQ ID No: 44)
HCA-Ala-Lys-Pro-Ser-Tyr-Hyp-Thr-Tyr-Hyp-Lys---;
(SEQ ID No: 45)
HCA-Ala-Lys-Pro-Ser-Tyr-Hyp-Hyp-Thr-Tyr-Lys---;
(SEQ ID No: 46)
HCA-Lys-Pro-Ser-Tyr-Hyp-Hyp-Thr-Ala-Lys---;
(SEQ ID No: 47)
HCA-Lys-Pro-Ser-Tyr-Hyp-Thr-Ala-Hyp-Lys---;
(SEQ ID No: 48)
DOPA-Lys-Pro-Ser-Tyr-Hyp-Thr-Ala-Hyp-Lys---;
(SEQ ID No: 49)
DOPA-Lys-Pro-Ser-Tyr-Hyp-Hyp-Thr-Ala-Lys---;
(SEQ ID No: 50)
DOPA-Ala-Lys-Pro-Ser-Tyr-Hyp-Thr-Tyr-Hyp-Lys---;
(SEQ ID No: 51)
DOPA-Ala-Lys-Pro-Ser-Tyr-Hyp-Hyp-Thr-Tyr-Lys---;
(SEQ ID No: 64)
Ala-Lys-Pro-Ser-DOPA-Hyp-Thr-DOPA-Hyp-Lys---;
(SEQ ID No: 65)
Ala-Lys-Pro-Ser-DOPA-Hyp-Hyp-Thr-DOPA-Lys---;
(SEQ ID No: 66)
Lys-Ala-Lys-Pro-Ser-DOPA-Hyp-Hyp-Thr-DOPA---;
(SEQ ID No: 67)
Lys-Ala-Lys-Hyp-Ser-DOPA-Hyp-Hyp-Thr-DOPA---;
(SEQ ID No: 68)
Ala-Lys-Pro-Ser-DOPA-Hyp-Hyp-Thr-DOPA-Lys-Ala-lys-Pro-Ser-DOPA-Hyp-Hyp-Thr-
DOPA-Lys---;
(SEQ ID No: 69)
Ala-Lys-Pro-Ser-DOPA-Hyp-Hyp-Thr-DOPA-Lys-Ala-Lys-Pro-Ser-DOPA-Hyp-Hyp-Thr-
DOPA-Lys-Ala-Lys-Pro-Ser-DOPA-Hyp-Hyp-Thr-DOPA-Lys-Ala-Lys-Pro-Ser-DOPA-Hyp-
Hyp-Thr-DOPA-Lys-Ala-Lys-Pro-Ser-DOPA-Hyp-Hyp-Thr-DOPA-Lys---;
(SEQ ID No: 93)
HCA-Ala-Lys-Pro-Ser-DOPA-Hyp-Thr-DOPA-Hyp-Lys---;
(SEQ ID No: 94)
HCA-Ala-Lys-Pro-Ser-DOPA-Hyp-Hyp-Thr-DOPA-Lys---;
(SEQ ID No: 95)
HCA-Lys-Pro-Ser-DOPA-Hyp-Thr-Ala-Hyp-Lys---;
(SEQ ID No: 96)
HCA-Lys-Pro-Ser-DOPA-Hyp-Hyp-Thr-Ala-Lys---;
(SEQ ID No: 97)
DOPA-Lys-Pro-Ser-DOPA-Hyp-Thr-Ala-Hyp-Lys---;
(SEQ ID No: 98)
DOPA-Lys-Pro-Ser-DOPA-Hyp-Hyp-Thr-Ala-Lys---;
(SEQ ID No: 99)
DOPA-Ala-Lys-Pro-Ser-DOPA-Hyp-Thr-DOPA-Hyp-Lys---;
(SEQ ID No: 100)
DOPA-Ala-Lys-Pro-Ser-DOPA-Hyp-Hyp-Thr-Tyr-Lys---;
and
(SEQ ID No: 101)
DOPA-Ala-Lys-Pro-Ser-DOPA-Hyp-Hyp-Thr-DOPA-Lys---.

36. A compound as claimed in any one of claims 33 to 35, wherein A and B both represent Z and both Z groups represent:
(SEQ ID No: 44)
HCA-Ala-Lys-Pro-Ser-Tyr-Hyp-Thr-Tyr-Hyp-Lys---,
(SEQ ID No: 47)
HCA-Lys-Pro-Ser-Tyr-Hyp-Thr-Ala-Hyp-Lys---,
(SEQ ID No: 50)
DOPA-Ala-Lys-Pro-Ser-Tyr-Hyp-Thr-Tyr-Hyp-Lys---,
(SEQ ID No: 64)
Ala-Lys-Pro-Ser-DOPA-Hyp-Thr-DOPA-Hyp-Lys---,
(SEQ ID No: 66)
Lys-Ala-Lys-Pro-Ser-DOPA-Hyp-Hyp-Thr-DOPA---
(SEQ ID No: 93)
HCA-Ala-Lys-Pro-Ser-DOPA-Hyp-Thr-DOPA-Hyp-Lys---,
(SEQ ID No: 98)
DOPA-Lys-Pro-Ser-DOPA-Hyp-Hyp-Thr-Ala-Lys---,
(SEQ ID No: 99)
DOPA-Ala-Lys-Pro-Ser-DOPA-Hyp-Thr-DOPA-Hyp-Lys---,
(SEQ ID No: 14)
Ala-Lys-Pro-Ser-Tyr-Hyp-Thr-Tyr-Hyp-Lys---,
(SEQ ID No: 15)
Ala-Lys-Pro-Ser-Tyr-Hyp-Thr-DOPA-Hyp-Lys---;
(SEQ ID No: 2)
Ala-Lys-Pro-Ser-Tyr-Hyp-Hyp-Thr-Tyr-Lys---,
or
(SEQ ID No: 1)
Ala-Lys-Pro-Ser-Tyr-Hyp-Hyp-Thr-DOPA-Lys---.

37. A compound as claimed in any one of claims 33 to 35, wherein A and B both represent A¹-W¹-B¹, A¹ and B¹ coin represent Z and both Z groups represent:
(SEQ ID No: 14)
Ala-Lys-Pro-Ser-Tyr-Hyp-Thr-Tyr-Hyp-Lys---,
(SEQ ID No: 15)
Ala-Lys-Pro-Ser-Tyr-Hyp-Thr-DOPA-Hyp-Lys---,
(SEQ ID No: 38)
Lys-Ala-Lys-Pro-Ser-Tyr-Hyp-Hyp-Thr-DOPA---,
(SEQ ID No: 44)
HCA-Ala-Lys-Pro-Ser-Tyr-Hyp-Thr-Tyr-Hyp-Lys---,
(SEQ ID No: 45)
HCA-Ala-Lys-Pro-Ser-Tyr-Hyp-Hyp-Thr-Tyr-Lys---,
(SEQ ID No: 50)
DOPA-Ala-Lys-Pro-Ser-Tyr-Hyp-Thr-Tyr-Hyp-Lys---,
(SEQ ID No: 64)
Ala-Lys-Pro-Ser-DOPA-Hyp-Thr-DOPA-Hyp-Lys---,
(SEQ ID No: 65)
Ala-Lys-Pro-Ser-DOPA-Hyp-Hyp-Thr-DOPA-Lys---,
(SEQ ID No: 37)
Lys-Ala-Lys-Hyp-Ser-Tyr-Hyp-Hyp-Thr-DOPA---,
(SEQ ID No: 95)
HCA-Lys-Pro-Ser-DOPA-Hyp-Thr-Ala-Hyp-Lys---,
(SEQ ID No: 97)
DOPA-Lys-Pro-Ser-DOPA-Hyp-Thr-Ala-Hyp-Lys---,
(SEQ ID No: 99)
DOPA-Ala-Lys-Pro-Ser-DOPA-Hyp-Thr-DOPA-Hyp-Lys---,
(SEQ ID No: 2)
Ala-Lys-Pro-Ser-Tyr-Hyp-Hyp-Thr-Tyr-Lys---
or
(SEQ ID NO: 1)
Ala-Lys-Pro-Ser-Tyr-Hyp-Hyp-Thr-DOPA-Lys---

38. A compound as claimed in any one of claims 33 to 35, wherein A and B both represent A¹-Q¹-B¹, A¹ and B¹ both represent A²-Q²-B², A² and B² both represent Z and both Z groups represent:
(SEQ ID No: 14)
Ala-Lys-Pro-Ser-Tyr-Hyp-Thr-Tyr-Hyp-Lys---,
(SEQ ID No: 15)
Ala-Lys-Pro-Ser-Tyr-Hyp-Thr-DOPA-Hyp-Lys---,
(SEQ ID No: 44)
HCA-Ala-Lys-Pro-Ser-Tyr-Hyp-Thr-Tyr-Hyp-Lys---,
(SEQ ID No: 50)
DOPA-Ala-Lys-Pro-Ser-Tyr-Hyp-Thr-Tyr-Hyp-Lys---,
(SEQ ID No: 65)
Ala-Lys-Pro-Ser-DOPA-Hyp-Hyp-Thr-DOPA-Lys---,
(SEQ ID No: 99)
DOPA-Ala-Lys-Pro-Ser-DOPA-Hyp-Thr-DOPA-Hyp-Lys---,
(SEQ ID No: 51)
DOPA-Ala-Lys-Pro-Ser-Tyr-Hyp-Hyp-Thr-Tyr-Lys---,
(SEQ ID No: 2)
Ala-Lys-Pro-Ser-Tyr-Hyp-Hyp-Thr-Tyr-Lys---,
or
(SEQ ID NO: 1)
Ala-Lys-Pro-Ser-Tyr-Hyp-Hyp-Thr-DOPA-Lys---

39. A compound as claimed in claim 38, wherein both Z groups represent Ala-Lys-Pro-Ser-Tyr-Hyp-Hyp-Thr-DOPA-Lys--- (SEQ ID No: 1).

40. A compound as claimed in any one of claims 33 to 35, wherein A and B both represent A¹-Q¹-B¹, A¹ and B¹ both represent A²-Q²-B², A² and B² both represent Z-Q³-Z and Z groups represent:
(SEQ ID No: 2)
Ala-Lys-Pro-Ser-Tyr-Hyp-Hyp-Thr-Tyr-Lys---,
or
(SEQ ID NO: 1)
Ala-Lys-Pro-Ser-Tyr-Hyp-Hyp-Thr-DOPA-Lys---.

41. A compound as defined in any one of the preceding claims, for use in human or animal medicine.

42. A compound as defined in any one of claims 1 to 40, for use as a pharmaceutical.

43. A pharmaceutical formulation comprising a compound as defined in any one of claims 1 to 40 and a pharmaceutically-acceptable adjuvant, diluent or carrier.

44. A pharmaceutical formulation as claimed in claim 43 that is suitable for, adapted for, and/or packaged and presented for administration by injection, wherein the pharmaceutically-acceptable adjuvant, diluent or carrier is an injectable adjuvant, diluent or carrier.

45. A compound as defined in any one of claims 1 to 40, or a formulation as claimed in a claim 43 or claim 44, for use in the treatment of a cancer.

46. The use of a compound as defined in any one of claims 1 to 40, or a formulation as claimed in a claim 43 or claim 44, for the manufacture a medicament for the treatment of a cancer.

47. A method of treatment of a cancer, which method comprises the administration of a compound as defined in any one of claims 1 to 40, or a formulation as claimed in a claim 43 or claim 44, to a patient in need of such treatment.

48. A compound as defined in any one of claims 1 to 40, or a formulation as claimed in a claim 43 or claim 44, for use in the imaging and/or the diagnosis of a cancer.

49. The use of a compound as defined in any one of claims 1 to 40, or a formulation as claimed in a claim 43 or claim 44, for the manufacture a medicament for the imaging and/or the diagnosis of a cancer.

50. A method of imaging and/or the diagnosis of a cancer, which method comprises the administration of a compound as defined in any one of claims 1 to 40, or a formulation as claimed in a claim 43 or claim 44, to a patient in need of such imaging and/or diagnosis.

51. A compound as defined in any one of claims 1 to 40, or a formulation as claimed in a claim 43 or claim 44, for use in a method of medical imaging.

52. The use of a compound as defined in any one of claims 1 to 40, or a formulation as claimed in a claim 43 or claim 44, for the manufacture a diagnostic medicament for a method of medical imaging.

53. A method of medical imaging, which method comprises the administration of a compound as defined in any one of claims 1 to 40, or a formulation as claimed in a claim 43 or claim 44, to a patient in need of such imaging.


SEQUENCE LISTING (182 sequences)

SEQ ID NO: 1 - protein, 10 aa, Unknown
  mefp-1 decapeptide
  SITE 6,7: Wherein Xaa is hydroxyproline
  SITE 9: Wherein Xaa is 3,4-dihydroxyphenylalanine
AKPSYXXTXK

SEQ ID NO: 2 - protein, 10 aa, Unknown
  mefp-1 decapeptide analogue
  SITE 6,7: Wherein Xaa is hydroxyproline
AKPSYXXTYK

SEQ ID NO: 3 - protein, 24 aa, Artificial Sequence
  REPEAT 1..12: Wherein the sequence is absent or is repeated between 1 and 4 times
  Peptide component
  VARIANT 1,13: Wherein Xaa absent or is selected from one or more of the group lysine, alanine, 3,4-dihydroxyphenylalanine, and a 3,4-dihydrocinnamic acid (HCA) residue, provided that, when present, the HCA residue is located at the N-terminus of the peptide
  VARIANT 2,14: Wherein Xaa is selected from one or more of the group lysine, alanine, 3,4-dihydroxyphenylalanine, and a 3,4-dihydrocinnamic acid (HCA) residue, provided that, when present, the HCA residue is located at the N-terminus of the peptide
  VARIANT 4,16: Wherein Xaa is proline, hydroxyproline, or dihydroxyproline
  VARIANT 6,18: Wherein Xaa is tyrosine or 3,4-dihydroxyphenylalanine
  VARIANT 7,19: Wherein Xaa is serine, proline, hydroxyproline or dihydroxyproline
  VARIANT 8,20: Wherein Xaa is selected from the group lysine, alanine, proline, hydroxyproline, dihydroxyproline, threonine, 3,4-dihydroxyphenylalanine and tyrosine
  VARIANT 9,10,11,12,21,22,23,24: Wherein Xaa is absent or is selected from the group lysine, alanine, proline, hydroxyproline, dihydroxyproline, threonine, 3,4-dihydroxyphenylalanine and tyrosine
XXKXSXXXXXXXXXKXSXXXXXXX

SEQ ID NO: 4 - protein, 23 aa, Artificial Sequence
  REPEAT 1..11: Wherein the sequence is repeated between 1 and 4 times
  Peptide component
  VARIANT 3,14: Wherein Xaa is proline, hydroxyproline, or dihydroxyproline
  VARIANT 5,16: Wherein Xaa is tyrosine or 3,4-dihydroxyphenylalanine
  VARIANT 6,17: Wherein Xaa is serine, proline, hydroxyproline or dihydroxyproline
  VARIANT 7,18: Wherein Xaa is selected from the group lysine, alanine, proline, hydroxyproline, dihydroxyproline, threonine, 3,4-dihydroxyphenylalanine and tyrosine
  VARIANT 8,9,10,11,19,20,21,22: Wherein Xaa is absent or is selected from the group lysine, alanine, proline, hydroxyproline, dihydroxyproline, threonine, 3,4-dihydroxyphenylalanine and tyrosine
  VARIANT 23: Wherein Xaa is absent or is 3,4-dihydroxyphenylalanine or dopamine or a dopamine fragment
AKXSXXXXXXXAKXSXXXXXXXX

SEQ ID NO: 5 - protein, 14 aa, Artificial Sequence
  Peptide component
  VARIANT 1..3: Wherein Xaa is absent or is selected from serine, lysine, alanine, 3,4-dihydroxyphenylalanine and a 3,4-dihydrocinnamic acid (HCA) residue, provided that, when present, the HCA residue is located at the N-terminus of the peptide sequence
  VARIANT 5: Wherein Xaa is serine, proline, hydroxyproline or dihydroxyproline
  VARIANT 7: Wherein Xaa is tyrosine, 3,4-dihydroxyphenylalanine or is absent
  VARIANT 8: Wherein Xaa is serine, proline, hydroxyproline or dihydroxyproline
  VARIANT 9: Wherein Xaa is selected from the group lysine, alanine, proline, hydroxyproline, dihydroxyproline, threonine, 3,4-dihydroxyphenylalanine and tyrosine
  VARIANT 10..13: Wherein Xaa is absent or is selected from the group lysine, alanine, proline, hydroxyproline, dihydroxyproline, threonine, 3,4-dihydroxyphenylalanine and tyrosine
  VARIANT 14: Wherein Xaa is absent or is 3,4-dihydroxyphenylalanine or dopamine or a dopamine fragment
XXXKXSXXXXXXXX

SEQ ID NO: 6 - protein, 20 aa, Artificial Sequence
  Peptide component
  SITE 7,17: Wherein Xaa is hydroxyproline
AKPSYSXTYKAKPSYSXTYK

SEQ ID NO: 7 - protein, 50 aa, Artificial Sequence
  Peptide component
  SITE 6,7,16,17,26,27,36,37,46,47: Wherein Xaa is hydroxyproline
AKPSYXXTYKAKPSYXXTYKAKPSYXXTYKAKPSYXXTYKAKPSYXXTYK

SEQ ID NO: 8 - protein, 20 aa, Artificial Sequence
  Peptide component
  SITE 5,9,15,19: Wherein Xaa is 3,4-dihydroxyphenylalanine
  SITE 6,7,16,17: Wherein Xaa is hydroxyproline
AKPSXXXTXKAKPSXXXTXK

SEQ ID NO: 9 - protein, 50 aa, Artificial Sequence
  Peptide component
  SITE 5,9,15,19,25,29,35,39,45,49: Wherein Xaa is 3,4-dihydroxyphenylalanine
  SITE 6,7,16,17,26,27,36,37,46,47: Wherein Xaa is hydroxyproline
AKPSXXXTXKAKPSXXXTXKAKPSXXXTXKAKPSXXXTXKAKPSXXXTXK

SEQ ID NO: 10 - protein, 20 aa, Artificial Sequence
  Peptide component
  SITE 6,7,16,17: Wherein Xaa is hydroxyproline
  SITE 9,19: Wherein Xaa is 3,4-dihydroxyphenylalanine
AKPSYXXTXKAKPSYXXTXK

SEQ ID NO: 11 - protein, 30 aa, Artificial Sequence
  Peptide component
  SITE 6,7,16,17,26,27: Wherein Xaa is hydroxyproline
  SITE 9,19,29: Wherein Xaa is 3,4-dihydroxyphenylalanine
AKPSYXXTXKAKPSYXXTXKAKPSYXXTXK

SEQ ID NO: 12 - protein, 40 aa, Artificial Sequence
  Peptide component
  SITE 6,7,16,17,26,27,36,37: Wherein Xaa is hydroxyproline
  SITE 9,19,29,39: Wherein Xaa is 3,4-dihydroxyphenylalanine
AKPSYXXTXKAKPSYXXTXKAKPSYXXTXKAKPSYXXTXK

SEQ ID NO: 13 - protein, 50 aa, Artificial Sequence
  Peptide component
  SITE 6,7,16,17,26,27,36,37,46,47: Wherein Xaa is hydroxyproline
  SITE 9,19,29,39,49: Wherein Xaa is 3,4-dihydroxyphenylalanine
AKPSYXXTXKAKPSYXXTXKAKPSYXXTXKAKPSYXXTXKAKPSYXXTXK

SEQ ID NO: 14 - protein, 10 aa, Artificial Sequence
  Peptide component
  SITE 6,9: Wherein Xaa is hydroxyproline
AKPSYXTYXK

SEQ ID NO: 15 - protein, 10 aa, Artificial Sequence
  Peptide component
  SITE 6,9: Wherein Xaa is hydroxyproline
  SITE 8: Wherein Xaa is 3,4-dihydroxyphenylalanine
AKPSYXTXXK

SEQ ID NO: 16 - protein, 11 aa, Artificial Sequence
  Peptide component
  SITE 6,9: Wherein Xaa is hydroxyproline
  SITE 11: Wherein Xaa is 3,4-dihydroxyphenylalanine
AKPSYXTYXKX

SEQ ID NO: 17 - protein, 20 aa, Artificial Sequence
  Peptide component
  SITE 7,17: Wherein Xaa is hydroxyproline
AKPSYSXTYKAKPSYSXTYK

SEQ ID NO: 18 - protein, 50 aa, Artificial Sequence; identical to SEQ ID NO: 7
  Peptide component
  SITE 6,7,16,17,26,27,36,37,46,47: Wherein Xaa is hydroxyproline

SEQ ID NO: 19 - protein, 11 aa, Artificial Sequence
  Peptide component
  SITE 6,9: Wherein Xaa is hydroxyproline
  SITE 11: Wherein Xaa is 3,4-dihydroxyphenylalanine
AKPSYXTYXKX

SEQ ID NO: 20 - protein, 11 aa, Artificial Sequence
  Peptide component
  SITE 6,9: Wherein Xaa is hydroxyproline
  SITE 11: Wherein Xaa is dopamine or a dopamine fragment
AKPSYXTYXKX

SEQ ID NO: 21 - protein, 11 aa, Artificial Sequence
  Peptide component
  SITE 6,7: Wherein Xaa is hydroxyproline
  SITE 11: Wherein Xaa is dopamine or a dopamine fragment
AKPSYXXTYKX

SEQ ID NO: 22 - protein, 10 aa, Artificial Sequence
  Peptide component
  SITE 9: Wherein Xaa is 3,4-dihydroxyphenylalanine
AKPSYPPTXK

SEQ ID NO: 23 - protein, 10 aa, Artificial Sequence
  Peptide component
AKPSYPTYPK

SEQ ID NO: 24 - protein, 10 aa, Artificial Sequence
  Peptide component
  SITE 8: Wherein Xaa is 3,4-dihydroxyphenylalanine
AKPSYPTXPK

SEQ ID NO: 25 - protein, 10 aa, Artificial Sequence
  Peptide component
  SITE 7: Wherein Xaa is hydroxyproline
AKPSYPXTYK

SEQ ID NO: 26 - protein, 10 aa, Artificial Sequence
  Peptide component
  SITE 7: Wherein Xaa is hydroxyproline
  SITE 9: Wherein Xaa is 3,4-dihydroxyphenylalanine
AKPSYPXTXK

SEQ ID NO: 27 - protein, 10 aa, Artificial Sequence
  Peptide component
  SITE 6: Wherein Xaa is hydroxyproline
AKPSYXPTYK

SEQ ID NO: 28 - protein, 10 aa, Artificial Sequence
  Peptide component
  SITE 6: Wherein Xaa is hydroxyproline
  SITE 9: Wherein Xaa is 3,4-dihydroxyphenylalanine
AKPSYXPTXK

SEQ ID NO: 29 - protein, 10 aa, Artificial Sequence
  SITE 1: Wherein Ala is optionally N-terminally modified with a molecule selected from DOTA, NOTA, TETA, ATSM, PTSM, and CB-TE2A, or is not so modified.
  Peptide component
  SITE 5,8: Wherein Tyr is optionally Iodinated with Iodine 125 or is not so modified.
  SITE 6,9: Wherein Xaa is hydroxyproline
AKPSYXTYXK

SEQ ID NO: 30 - protein, 10 aa, Artificial Sequence
  SITE 1: Wherein Ala is optionally N-terminally modified with a DOTA molecule or is not so modified.
  Peptide component
  SITE 5: Wherein Tyr is optionally Iodinated with Iodine 125 or Iodine 131, or is not so modified.
  SITE 6,9: Wherein Xaa is hydroxyproline
  SITE 8: Wherein Xaa is 3,4-dihydroxyphenylalanine
  SITE 8: Wherein Xaa is optionally Iodinated with Iodine 125 or Iodine 131, or is not so modified.
AKPSYXTXXK

SEQ ID NO: 31 - protein, 11 aa, Artificial Sequence
  Peptide component
  SITE 6,7: Wherein Xaa is hydroxyproline
  SITE 11: Wherein Xaa is 3,4-dihydroxyphenylalanine
AKPSYXXTYKX

SEQ ID NO: 32 - protein, 11 aa, Artificial Sequence
  Peptide component
  SITE 6,7: Wherein Xaa is hydroxyproline
  SITE 9,11: Wherein Xaa is 3,4-dihydroxyphenylalanine
AKPSYXXTXKX

SEQ ID NO: 33 - protein, 11 aa, Artificial Sequence
  Peptide component
  SITE 6,7: Wherein Xaa is hydroxyproline
  SITE 9: Wherein Xaa is 3,4-dihydroxyphenylalanine
  SITE 11: Wherein Xaa is dopamine or a dopamine fragment
AKPSYXXTXKX

SEQ ID NO: 34 - protein, 11 aa, Artificial Sequence
  Peptide component
  SITE 6,9: Wherein Xaa is hydroxyproline
  SITE 8,11: Wherein Xaa is 3,4-dihydroxyphenylalanine
AKPSYXTXXKX

SEQ ID NO: 35 - protein, 11 aa, Artificial Sequence
  Peptide component
  SITE 6,9: Wherein Xaa is hydroxyproline
  SITE 8: Wherein Xaa is 3,4-dihydroxyphenylalanine
  SITE 11: Wherein Xaa is dopamine or a dopamine fragment
AKPSYXTXXKX

SEQ ID NO: 36 - protein, 10 aa, Artificial Sequence
  Peptide component
  SITE 7,8: Wherein Xaa is hydroxyproline
KAKPSYXXTY

SEQ ID NO: 37 - protein, 10 aa, Artificial Sequence
  Peptide component
  SITE 4,7,8: Wherein Xaa is hydroxyproline
  SITE 10: Wherein Xaa is 3,4-dihydroxyphenylalanine
KAKXSYXXTX

SEQ ID NO: 38 - protein, 10 aa, Artificial Sequence
  Peptide component
  SITE 7,8: Wherein Xaa is hydroxyproline
  SITE 10: Wherein Xaa is 3,4-dihydroxyphenylalanine
KAKPSYXXTX

SEQ ID NO: 39 - protein, 10 aa, Artificial Sequence
  Peptide component
  SITE 7,8: Wherein Xaa is hydroxyproline
  SITE 10: Wherein Xaa is 3,4-dihydroxyphenylalanine
KAKPSYXXTX

SEQ ID NO: 40 - protein, 10 aa, Artificial Sequence
  Peptide component
  SITE 7,8: Wherein Xaa is hydroxyproline
  SITE 10: Wherein Xaa is dopamine or a dopamine fragment
KAKPSYXXTX

SEQ ID NO: 41 - protein, 10 aa, Artificial Sequence
  Peptide component
  SITE 4,7,8: Wherein Xaa is hydroxyproline
  SITE 10: Wherein Xaa is 3,4-dihydroxyphenylalanine
KAKXSYXXTX

SEQ ID NO: 42 - protein, 10 aa, Artificial Sequence
  Peptide component
  SITE 4,7,8: Wherein Xaa is hydroxyproline
KAKXSYXXTY

SEQ ID NO: 43 - protein, 10 aa, Artificial Sequence
  Peptide component
  SITE 7,8: Wherein Xaa is hydroxyproline
KAKPSYXXTY

SEQ ID NO: 44 - protein, 10 aa, Artificial Sequence
  SITE 1: Wherein Ala is N-terminally modified with a 3,4-Dihydrocinnamic acid group
  Peptide component
  SITE 6,9: Wherein Xaa is hydroxyproline
AKPSYXTYXK

SEQ ID NO: 45 - protein, 10 aa, Artificial Sequence
  SITE 1: Wherein Ala is N-terminally modified with a 3,4-Dihydrocinnamic acid group
  Peptide component
  SITE 6,7: Wherein Xaa is hydroxyproline
AKPSYXXTYK

SEQ ID NO: 46 - protein, 9 aa, Artificial Sequence
  SITE 1: Wherein Lys is N-terminally modified with a 3,4-Dihydrocinnamic acid group
  Peptide component
  SITE 5,6: Wherein Xaa is hydroxyproline
KPSYXXTAK

SEQ ID NO: 47 - protein, 9 aa, Artificial Sequence
  SITE 1: Wherein Lys is N-terminally modified with a 3,4-Dihydrocinnamic acid group
  Peptide component
  SITE 5,8: Wherein Xaa is hydroxyproline
KPSYXTAXK

SEQ ID NO: 48 - protein, 10 aa, Artificial Sequence
  SITE 1: Wherein Xaa is 3,4-dihydroxyphenylalanine
  Peptide component
  SITE 6,9: Wherein Xaa is hydroxyproline
XKPSYXTAXK

SEQ ID NO: 49 - protein, 10 aa, Artificial Sequence
  SITE 1: Wherein Xaa is 3,4-dihydroxyphenylalanine
  Peptide component
  SITE 6,7: Wherein Xaa is hydroxyproline
XKPSYXXTAK

SEQ ID NO: 50 - protein, 11 aa, Artificial Sequence
  SITE 1: Wherein Xaa is 3,4-dihydroxyphenylalanine
  Peptide component
  SITE 7,10: Wherein Xaa is hydroxyproline
XAKPSYXTYXK

SEQ ID NO: 51 - protein, 11 aa, Artificial Sequence
  SITE 1: Wherein Xaa is 3,4-dihydroxyphenylalanine
  Peptide component
  SITE 7,8: Wherein Xaa is hydroxyproline
XAKPSYXXTYK

SEQ ID NO: 52 - protein, 10 aa, Artificial Sequence
  SITE 1: Wherein Xaa is 3,4-dihydroxyphenylalanine
  Peptide component
  SITE 6,9: Wherein Xaa is hydroxyproline
XKPSYXTAXK

SEQ ID NO: 53 - protein, 10 aa, Artificial Sequence
  SITE 1: Wherein Xaa is 3,4-dihydroxyphenylalanine
  Peptide component
  SITE 6,7: Wherein Xaa is hydroxyproline
XKPSYXXTAK

SEQ ID NO: 54 - protein, 11 aa, Artificial Sequence
  SITE 1: Wherein Xaa is 3,4-dihydroxyphenylalanine
  Peptide component
  SITE 7,10: Wherein Xaa is hydroxyproline
XAKPSYXTYXK

SEQ ID NO: 55 - protein, 11 aa, Artificial Sequence
  SITE 1: Wherein Xaa is 3,4-dihydroxyphenylalanine
  Peptide component
  SITE 7,8: Wherein Xaa is hydroxyproline
XAKPSYXXTYK

SEQ ID NO: 56 - protein, 11 aa, Artificial Sequence
  SITE 1,9: Wherein Xaa is 3,4-dihydroxyphenylalanine
  Peptide component
  SITE 7,10: Wherein Xaa is hydroxyproline
XAKPSYXTXXK

SEQ ID NO: 57 - protein, 11 aa, Artificial Sequence
  SITE 1,10: Wherein Xaa is 3,4-dihydroxyphenylalanine
  Peptide component
  SITE 7,8: Wherein Xaa is hydroxyproline
XAKPSYXXTXK

SEQ ID NO: 58 - protein, 9 aa, Artificial Sequence
  SITE 1: Wherein Lys is N-terminally modified with a 3,4-Dihydrocinnamic acid group
  Peptide component
  SITE 5,6: Wherein Xaa is hydroxyproline
KPSYXXTAK

SEQ ID NO: 59 - protein, 10 aa, Artificial Sequence
  SITE 1: Wherein Ala is N-terminally modified with a 3,4-Dihydrocinnamic acid group
  Peptide component
  SITE 6,9: Wherein Xaa is hydroxyproline
AKPSYXTYXK

SEQ ID NO: 60 - protein, 10 aa, Artificial Sequence
  SITE 1: Wherein Ala is N-terminally modified with a 3,4-Dihydrocinnamic acid group
  Peptide component
  SITE 6,9: Wherein Xaa is hydroxyproline
  SITE 8: Wherein Xaa is 3,4-dihydroxyphenylalanine
AKPSYXTXXK

SEQ ID NO: 61 - protein, 10 aa, Artificial Sequence
  SITE 1: Wherein Ala is N-terminally modified with a 3,4-Dihydrocinnamic acid group
  Peptide component
  SITE 6,7: Wherein Xaa is hydroxyproline
AKPSYXXTYK

SEQ ID NO: 62 - protein, 10 aa, Artificial Sequence
  SITE 1: Wherein Ala is N-terminally modified with a 3,4-Dihydrocinnamic acid group
  Peptide component
  SITE 6,7: Wherein Xaa is hydroxyproline
  SITE 9: Wherein Xaa is 3,4-dihydroxyphenylalanine
AKPSYXXTXK

SEQ ID NO: 63 - protein, 9 aa, Artificial Sequence
  SITE 1: Wherein Lys is N-terminally modified with a 3,4-Dihydrocinnamic acid group
  Peptide component
  SITE 5,8: Wherein Xaa is hydroxyproline
KPSYXTAXK

SEQ ID NO: 64 - protein, 10 aa, Artificial Sequence
  Peptide component
  SITE 5,8: Wherein Xaa is 3,4-dihydroxyphenylalanine
  SITE 6,9: Wherein Xaa is hydroxyproline
AKPSXXTXXK

SEQ ID NO: 65 - protein, 10 aa, Artificial Sequence
  Peptide component
  SITE 5,9: Wherein Xaa is 3,4-dihydroxyphenylalanine
  SITE 6,7: Wherein Xaa is hydroxyproline
AKPSXXXTXK

SEQ ID NO: 66 - protein, 10 aa, Artificial Sequence
  Peptide component
  SITE 6,10: Wherein Xaa is 3,4-dihydroxyphenylalanine
  SITE 7,8: Wherein Xaa is hydroxyproline
KAKPSXXXTX

SEQ ID NO: 67 - protein, 10 aa, Artificial Sequence
  Peptide component
  SITE 4,7,8: Wherein Xaa is hydroxyproline
  SITE 6,10: Wherein Xaa is 3,4-dihydroxyphenylalanine
KAKXSXXXTX

SEQ ID NO: 68 - protein, 20 aa, Artificial Sequence
  Peptide component
  SITE 5,9,15,19: Wherein Xaa is 3,4-dihydroxyphenylalanine
  SITE 6,7,16,17: Wherein Xaa is hydroxyproline
AKPSXXXTXKAKPSXXXTXK

SEQ ID NO: 69 - protein, 50 aa, Artificial Sequence; identical to SEQ ID NO: 9
  Peptide component
  SITE 5,9,15,19,25,29,35,39,45,49: Wherein Xaa is 3,4-dihydroxyphenylalanine
  SITE 6,7,16,17,26,27,36,37,46,47: Wherein Xaa is hydroxyproline

SEQ ID NO: 70 - protein, 10 aa, Artificial Sequence
  Peptide component
  SITE 6: Wherein Xaa is 3,4-dihydroxyphenylalanine
  SITE 7,8: Wherein Xaa is hydroxyproline
  SITE 10: Wherein Xaa is dopamine or a dopamine fragment
KAKPSXXXTX

SEQ ID NO: 71 - protein, 10 aa, Artificial Sequence
  Peptide component
  SITE 4,7,8: Wherein Xaa is hydroxyproline
  SITE 6,10: Wherein Xaa is 3,4-dihydroxyphenylalanine
KAKXSXXXTX

SEQ ID NO: 72 - protein, 10 aa, Artificial Sequence
  Peptide component
  SITE 6,10: Wherein Xaa is 3,4-dihydroxyphenylalanine
  SITE 7,8: Wherein Xaa is hydroxyproline
KAKPSXXXTX

SEQ ID NO: 73 - protein, 10 aa, Artificial Sequence
  Peptide component
  SITE 5: Wherein Xaa is 3,4-dihydroxyphenylalanine
AKPSXPPTYK

SEQ ID NO: 74 - protein, 10 aa, Artificial Sequence
  Peptide component
  SITE 5,9: Wherein Xaa is 3,4-dihydroxyphenylalanine
AKPSXPPTXK

SEQ ID NO: 75 - protein, 10 aa, Artificial Sequence
  Peptide component
  SITE 5: Wherein Xaa is 3,4-dihydroxyphenylalanine
AKPSXPTYPK

SEQ ID NO: 76 - protein, 10 aa, Artificial Sequence
  Peptide component
  SITE 5,8: Wherein Xaa is 3,4-dihydroxyphenylalanine
AKPSXPTXPK

SEQ ID NO: 77 - protein, 10 aa, Artificial Sequence
  Peptide component
  SITE 5: Wherein Xaa is 3,4-dihydroxyphenylalanine
  SITE 7: Wherein Xaa is hydroxyproline
AKPSXPXTYK

SEQ ID NO: 78 - protein, 10 aa, Artificial Sequence
  Peptide component
  SITE 5,9: Wherein Xaa is 3,4-dihydroxyphenylalanine
  SITE 7: Wherein Xaa is hydroxyproline
AKPSXPXTXK

SEQ ID NO: 79 - protein, 10 aa, Artificial Sequence
  Peptide component
  SITE 5: Wherein Xaa is 3,4-dihydroxyphenylalanine
  SITE 6: Wherein Xaa is hydroxyproline
AKPSXXPTYK

SEQ ID NO: 80 - protein, 10 aa, Artificial Sequence
  Peptide component
  SITE 5,9: Wherein Xaa is 3,4-dihydroxyphenylalanine
  SITE 6: Wherein Xaa is hydroxyproline
AKPSXXPTXK

SEQ ID NO: 81 - protein, 10 aa, Artificial Sequence
  Peptide component
  SITE 5: Wherein Xaa is 3,4-dihydroxyphenylalanine
  SITE 6,9: Wherein Xaa is hydroxyproline
AKPSXXTYXK

SEQ ID NO: 82 - protein, 10 aa, Artificial Sequence
  Peptide component
  SITE 5,8: Wherein Xaa is 3,4-dihydroxyphenylalanine
  SITE 6,9: Wherein Xaa is hydroxyproline
AKPSXXTXXK

SEQ ID NO: 83 - protein, 10 aa, Artificial Sequence
  Peptide component
  SITE 6: Wherein Xaa is 3,4-dihydroxyphenylalanine
  SITE 7,8: Wherein Xaa is hydroxyproline
KAKPSXXXTY

SEQ ID NO: 84 - protein, 10 aa, Artificial Sequence
  Peptide component
  SITE 4,7,8: Wherein Xaa is hydroxyproline
  SITE 6: Wherein Xaa is 3,4-dihydroxyphenylalanine
KAKXSXXXTY

SEQ ID NO: 85 - protein, 11 aa, Artificial Sequence
  Peptide component
  SITE 5,11: Wherein Xaa is 3,4-dihydroxyphenylalanine
  SITE 6,7: Wherein Xaa is hydroxyproline
AKPSXXXTYKX

SEQ ID NO: 86 - protein, 11 aa, Artificial Sequence
  Peptide component
  SITE 5,9,11: Wherein Xaa is 3,4-dihydroxyphenylalanine
  SITE 6,7: Wherein Xaa is hydroxyproline
AKPSXXXTXKX

SEQ ID NO: 87 - protein, 11 aa, Artificial Sequence
  Peptide component
  SITE 5: Wherein Xaa is 3,4-dihydroxyphenylalanine
  SITE 6,7: Wherein Xaa is hydroxyproline
  SITE 11: Wherein Xaa is dopamine or a dopamine fragment
AKPSXXXTYKX

SEQ ID NO: 88 - protein, 11 aa, Artificial Sequence
  Peptide component
  SITE 5,9: Wherein Xaa is 3,4-dihydroxyphenylalanine
  SITE 6,7: Wherein Xaa is hydroxyproline
  SITE 11: Wherein Xaa is dopamine or a dopamine fragment
AKPSXXXTXKX

SEQ ID NO: 89 - protein, 11 aa, Artificial Sequence
  Peptide component
  SITE 5,11: Wherein Xaa is 3,4-dihydroxyphenylalanine
  SITE 6,9: Wherein Xaa is hydroxyproline
AKPSXXTYXKX

SEQ ID NO: 90 - protein, 11 aa, Artificial Sequence
  Peptide component
  SITE 5,8,11: Wherein Xaa is 3,4-dihydroxyphenylalanine
  SITE 6,9: Wherein Xaa is hydroxyproline
AKPSXXTXXKX

SEQ ID NO: 91 - protein, 11 aa, Artificial Sequence
  Peptide component
  SITE 5: Wherein Xaa is 3,4-dihydroxyphenylalanine
  SITE 6,9: Wherein Xaa is hydroxyproline
  SITE 11: Wherein Xaa is dopamine or a dopamine fragment
AKPSXXTYXKX

SEQ ID NO: 92 - protein, 11 aa, Artificial Sequence
  Peptide component
  SITE 5,8: Wherein Xaa is 3,4-dihydroxyphenylalanine
  SITE 6,9: Wherein Xaa is hydroxyproline
  SITE 11: Wherein Xaa is dopamine or a dopamine fragment
AKPSXXTXXKX

SEQ ID NO: 93 - protein, 10 aa, Artificial Sequence
  SITE 1: Wherein Ala is N-terminally modified with a 3,4-Dihydrocinnamic acid group
  Peptide component
  SITE 5,8: Wherein Xaa is 3,4-dihydroxyphenylalanine
  SITE 6,9: Wherein Xaa is hydroxyproline
AKPSXXTXXK

SEQ ID NO: 94 - protein, 10 aa, Artificial Sequence
  SITE 1: Wherein Ala is N-terminally modified with a 3,4-Dihydrocinnamic acid group
  Peptide component
  SITE 5,9: Wherein Xaa is 3,4-dihydroxyphenylalanine
  SITE 6,7: Wherein Xaa is hydroxyproline
AKPSXXXTXK

SEQ ID NO: 95 - protein, 9 aa, Artificial Sequence
  SITE 1: Wherein Lys is N-terminally modified with a 3,4-Dihydrocinnamic acid group
  Peptide component
  SITE 4: Wherein Xaa is 3,4-dihydroxyphenylalanine
  SITE 5,8: Wherein Xaa is hydroxyproline
KPSXXTAXK

SEQ ID NO: 96 - protein, 9 aa, Artificial Sequence
  SITE 1: Wherein Lys is N-terminally modified with a 3,4-Dihydrocinnamic acid group
  Peptide component
  SITE 4: Wherein Xaa is 3,4-dihydroxyphenylalanine
  SITE 5,6: Wherein Xaa is hydroxyproline
KPSXXXTAK

SEQ ID NO: 97 - protein, 10 aa, Artificial Sequence
  SITE 1,5: Wherein Xaa is 3,4-dihydroxyphenylalanine
  Peptide component
  SITE 6,9: Wherein Xaa is hydroxyproline
XKPSXXTAXK

SEQ ID NO: 98 - protein, 10 aa, Artificial Sequence
  SITE 1,5: Wherein Xaa is 3,4-dihydroxyphenylalanine
  Peptide component
  SITE 6,7: Wherein Xaa is hydroxyproline
XKPSXXXTAK

SEQ ID NO: 99 - protein, 11 aa, Artificial Sequence
  SITE 1,6,9: Wherein Xaa is 3,4-dihydroxyphenylalanine
  Peptide component
  SITE 7,10: Wherein Xaa is hydroxyproline
XAKPSXXTXXK

SEQ ID NO: 100 - protein, 11 aa, Artificial Sequence
  SITE 1,6: Wherein Xaa is 3,4-dihydroxyphenylalanine
  Peptide component
  SITE 7,8: Wherein Xaa is hydroxyproline
XAKPSXXXTYK

SEQ ID NO: 101 - protein, 11 aa, Artificial Sequence
  SITE 1,6,10: Wherein Xaa is 3,4-dihydroxyphenylalanine
  Peptide component
  SITE 7,8: Wherein Xaa is hydroxyproline
XAKPSXXXTXK

SEQ ID NO: 102 - protein, 10 aa, Artificial Sequence
  SITE 1,5: Wherein Xaa is 3,4-dihydroxyphenylalanine
  Peptide component
  SITE 6,9: Wherein Xaa is hydroxyproline
XKPSXXTAXK

SEQ ID NO: 103 - protein, 10 aa, Artificial Sequence
  SITE 1,5: Wherein Xaa is 3,4-dihydroxyphenylalanine
  Peptide component
  SITE 6,7: Wherein Xaa is hydroxyproline
XKPSXXXTAK

SEQ ID NO: 104 - protein, 11 aa, Artificial Sequence
  SITE 1,6,9: Wherein Xaa is 3,4-dihydroxyphenylalanine
  Peptide component
  SITE 7,10: Wherein Xaa is hydroxyproline
XAKPSXXTXXK

SEQ ID NO: 105 - protein, 11 aa, Artificial Sequence
  SITE 1,6: Wherein Xaa is 3,4-dihydroxyphenylalanine
  Peptide component
  SITE 7,8: Wherein Xaa is hydroxyproline
XAKPSXXXTYK

SEQ ID NO: 106 - protein, 11 aa, Artificial Sequence
  SITE 1,6,10: Wherein Xaa is 3,4-dihydroxyphenylalanine
  Peptide component
  SITE 7,8: Wherein Xaa is hydroxyproline
XAKPSXXXTXK

SEQ ID NO: 107 - protein, 10 aa, Artificial Sequence
  SITE 1: Wherein Ala is N-terminally modified with a 3,4-Dihydrocinnamic acid group
  Peptide component
  SITE 5,8: Wherein Xaa is 3,4-dihydroxyphenylalanine
  SITE 6,9: Wherein Xaa is hydroxyproline
AKPSXXTXXK

SEQ ID NO: 108 - protein, 10 aa, Artificial Sequence
  SITE 1: Wherein Ala is N-terminally modified with a 3,4-Dihydrocinnamic acid group
  Peptide component
  SITE 5,9: Wherein Xaa is 3,4-dihydroxyphenylalanine
  SITE 6,7: Wherein Xaa is hydroxyproline
AKPSXXXTXK

SEQ ID NO: 109 - protein, 9 aa, Artificial Sequence
  SITE 1: Wherein Lys is N-terminally modified with a 3,4-Dihydrocinnamic acid group
  Peptide component
  SITE 4: Wherein Xaa is 3,4-dihydroxyphenylalanine
  SITE 5,8: Wherein Xaa is hydroxyproline
KPSXXTAXK

SEQ ID NO: 110 - protein, 9 aa, Artificial Sequence
  SITE 1: Wherein Lys is N-terminally modified with a 3,4-Dihydrocinnamic acid group
  Peptide component
  SITE 4: Wherein Xaa is 3,4-dihydroxyphenylalanine
  SITE 5,6: Wherein Xaa is hydroxyproline
KPSXXXTAK

SEQ ID NO: 111 - protein, 11 aa, Artificial Sequence
  SITE 1,6: Wherein Xaa is 3,4-dihydroxyphenylalanine
  Peptide component
  SITE 7,10: Wherein Xaa is hydroxyproline
XAKPSXXTYXK

SEQ ID NO: 112 - protein, 10 aa, Artificial Sequence
  SITE 1: Wherein Ala is N-terminally modified with a 3,4-Dihydrocinnamic acid group
  Peptide component
  SITE 5: Wherein Xaa is 3,4-dihydroxyphenylalanine
  SITE 6,9: Wherein Xaa is hydroxyproline
AKPSXXTYXK

SEQ ID NO: 113 - protein, 10 aa, Artificial Sequence
  SITE 1: Wherein Ala is N-terminally modified with a 3,4-Dihydrocinnamic acid group
  Peptide component
  SITE 5: Wherein Xaa is 3,4-dihydroxyphenylalanine
  SITE 6,7: Wherein Xaa is hydroxyproline
AKPSXXXTYK

SEQ ID NO: 114 - protein, 12 aa, Artificial Sequence
  Peptide component
  VARIANT 1: Wherein Xaa is optionally N-terminally modified with a 3,4-Dihydrocinnamic acid group or is not so modified
  VARIANT 1,2: Where Xaa is absent or is selected from the group serine, lysine, alanine and 3,4-dihydroxyphenylalanine
  VARIANT 4: Wherein Xaa is proline, hydroxyproline, or dihydroxyproline
  VARIANT 6: Wherein Xaa is tyrosine, 3,4-dihydroxyphenylalanine or is absent
  VARIANT 7: Wherein Xaa is serine, proline, hydroxyproline or dihydroxyproline
  VARIANT 8..10: Wherein Xaa is absent or is selected from the group lysine, alanine, proline, hydroxyproline, dihydroxyproline, threonine, 3,4-dihydroxyphenylalanine and tyrosine
  VARIANT 11: Wherein Xaa is proline, hydroxyproline, dihydroxyproline, threonine, 3,4-dihydroxyphenylalanine, or tyrosine
  VARIANT 12: Wherein Xaa is absent or is lysine
XXKXSXXXXXXX

SEQ ID NO: 115 - protein, 10 aa, Artificial Sequence
  SITE 1: Wherein Lys is optionally N-terminally modified with a DOTA molecule or is not so modified.
  Peptide component
  SITE 4: Wherein Tyr is optionally Iodinated with Iodine 125 or Iodine 131, or is not so modified.
  SITE 5,6,9: Wherein Xaa is hydroxyproline
  SITE 8: Wherein Xaa is 3,4-dihydroxyphenylalanine
  SITE 8: Wherein Xaa is optionally Iodinated with Iodine 125 or Iodine 131, or is not so modified.
KPSYXXTXXK

SEQ ID NO: 116 - protein, 10 aa, Artificial Sequence
  SITE 1: Wherein Lys is optionally N-terminally modified with a DOTA molecule or is not so modified.
  Peptide component
  SITE 4,8: Wherein Tyr is optionally Iodinated with Iodine 125 or Iodine 131, or is not so modified.
  SITE 5,6,9: Wherein Xaa is hydroxyproline
KPSYXXTYXK

SEQ ID NO: 117 - protein, 9 aa, Artificial Sequence
  SITE 1: Wherein Lys is optionally N-terminally modified with a DOTA molecule or is not so modified.
  Peptide component
  SITE 4: Wherein Tyr is optionally Iodinated with Iodine 125 or Iodine 131, or is not so modified.
  SITE 5,8: Wherein Xaa is hydroxyproline
  SITE 7: Wherein Xaa is 3,4-dihydroxyphenylalanine
  SITE 7: Wherein Xaa is optionally Iodinated with Iodine 125 or Iodine 131, or is not so modified.
KPSYXTXXK

SEQ ID NO: 118 - protein, 9 aa, Artificial Sequence
  SITE 1: Wherein Lys is optionally N-terminally modified with a DOTA molecule or is not so modified.
  Peptide component
  SITE 4,7: Wherein Tyr is optionally Iodinated with Iodine 125 or Iodine 131, or is not so modified.
  SITE 5,8: Wherein Xaa is hydroxyproline
KPSYXTYXK

SEQ ID NO: 119 - protein, 10 aa, Artificial Sequence
  SITE 1: Wherein Ser is optionally N-terminally modified with a DOTA molecule or is not so modified.
  Peptide component
  SITE 5: Wherein Tyr is optionally Iodinated with Iodine 125 or Iodine 131, or is not so modified.
  SITE 6,7: Wherein Xaa is hydroxyproline
  SITE 9: Wherein Xaa is 3,4-dihydroxyphenylalanine
  SITE 9: Wherein Xaa is optionally Iodinated with Iodine 125 or Iodine 131, or is not so modified.
SKPSYXXTXK

SEQ ID NO: 120 - protein, 10 aa, Artificial Sequence
  SITE 1: Wherein Ser is optionally N-terminally modified with a DOTA molecule or is not so modified.
  Peptide component
  SITE 5,9: Wherein Tyr is optionally Iodinated with Iodine 125 or Iodine 131, or is not so modified.
  SITE 6,7: Wherein Xaa is hydroxyproline
SKPSYXXTYK

SEQ ID NO: 121 - protein, 10 aa, Artificial Sequence
  SITE 1: Wherein Ser is optionally N-terminally modified with a DOTA molecule or is not so modified.
  Peptide component
  SITE 5: Wherein Tyr is optionally Iodinated with Iodine 125 or Iodine 131, or is not so modified.
  SITE 6,9: Wherein Xaa is hydroxyproline
  SITE 8: Wherein Xaa is 3,4-dihydroxyphenylalanine
  SITE 8: Wherein Xaa is optionally Iodinated with Iodine 125 or Iodine 131, or is not so modified.
SKPSYXTXXK

SEQ ID NO: 122 - protein, 10 aa, Artificial Sequence
  SITE 1: Wherein Ser is optionally N-terminally modified with a DOTA molecule or is not so modified.
  Peptide component
  SITE 5,8: Wherein Tyr is optionally Iodinated with Iodine 125 or Iodine 131, or is not so modified.
  SITE 6,9: Wherein Xaa is hydroxyproline
SKPSYXTYXK

SEQ ID NO: 123 - protein, 11 aa, Artificial Sequence
  SITE 1: Wherein Ala is optionally N-terminally modified with a DOTA molecule or is not so modified.
  Peptide component
  SITE 5: Wherein Tyr is optionally Iodinated with Iodine 125 or Iodine 131, or is not so modified.
  SITE 6,7,10: Wherein Xaa is hydroxyproline
  SITE 9: Wherein Xaa is 3,4-dihydroxyphenylalanine
  SITE 9: Wherein Xaa is optionally Iodinated with Iodine 125 or Iodine 131, or is not so modified.
AKPSYXXTXXK

SEQ ID NO: 124 - protein, 11 aa, Artificial Sequence
  SITE 1: Wherein Ala is optionally N-terminally modified with a molecule selected from DOTA, NOTA, TETA, ATSM, PTSM, and CB-TE2A, or is not so modified.
  Peptide component
  SITE 5,9: Wherein Tyr is optionally Iodinated with Iodine 125 or Iodine 131, or is not so modified.
  SITE 6,7,10: Wherein Xaa is hydroxyproline
AKPSYXXTYXK

SEQ ID NO: 125 - protein, 10 aa, Artificial Sequence
  Peptide component
  SITE 6,9: Wherein Xaa is hydroxyproline
  SITE 8: Wherein Xaa is 3,4-dihydroxyphenylalanine
AKPSYXTXXK

SEQ ID NO: 126 - protein, 10 aa, Artificial Sequence
  Peptide component
  SITE 6,9: Wherein Xaa is hydroxyproline
AKPSYXTYXK

SEQ ID NO: 127 - protein, 9 aa, Artificial Sequence
  Peptide component
  SITE 5,6,9: Wherein Xaa is hydroxyproline
  SITE 8: Wherein Xaa is 3,4-dihydroxyphenylalanine
KPSYXXTXX

SEQ ID NO: 128 - protein, 9 aa, Artificial Sequence
  Peptide component
  SITE 5,6,9: Wherein Xaa is hydroxyproline
KPSYXXTYX

SEQ ID NO: 129 - protein, 8 aa, Artificial Sequence
  Peptide component
  SITE 5,8: Wherein Xaa is hydroxyproline
  SITE 7: Wherein Xaa is 3,4-dihydroxyphenylalanine
KPSYXTXX

SEQ ID NO: 130 - protein, 8 aa, Artificial Sequence
  Peptide component
  SITE 5,8: Wherein Xaa is hydroxyproline
KPSYXTYX

SEQ ID NO: 131 - protein, 9 aa, Artificial Sequence
  Peptide component
  SITE 6,7: Wherein Xaa is hydroxyproline
SKPSYXXTY

SEQ ID NO: 132 - protein, 9 aa, Artificial Sequence
  Peptide component
  SITE 6,9: Wherein Xaa is hydroxyproline
  SITE 8: Wherein Xaa is 3,4-dihydroxyphenylalanine
SKPSYXTXX

SEQ ID NO: 133 - protein, 9 aa, Artificial Sequence
  Peptide component
  SITE 6,9: Wherein Xaa is hydroxyproline
SKPSYXTYX

SEQ ID NO: 134 - protein, 10 aa, Artificial Sequence
  Peptide component
  SITE 6,7,10: Wherein Xaa is hydroxyproline
  SITE 9: Wherein Xaa is 3,4-dihydroxyphenylalanine
AKPSYXXTXX

SEQ ID NO: 135 - protein, 10 aa, Artificial Sequence
  Peptide component
  SITE 6,7,10: Wherein Xaa is hydroxyproline
AKPSYXXTYX

SEQ ID NO: 136 - protein, 9 aa, Artificial Sequence
  Peptide component
  SITE 6,9: Wherein Xaa is hydroxyproline
  SITE 8: Wherein Xaa is 3,4-dihydroxyphenylalanine
AKPSYXTXX

SEQ ID NO: 137 - protein, 9 aa, Artificial Sequence
  Peptide component
  SITE 6,9: Wherein Xaa is hydroxyproline
AKPSYXTYX

SEQ ID NO: 138 - protein, 9 aa, Artificial Sequence
  Peptide component
  SITE 6,7: Wherein Xaa is hydroxyproline
  SITE 9: Wherein Xaa is 3,4-dihydroxyphenylalanine
SKPSYXXTX

SEQ ID NO: 139 - protein, 10 aa, Artificial Sequence
  SITE 1: Wherein Ala is N-terminally modified with a 3,4-Dihydrocinnamic acid group
  Peptide component
  SITE 6,7: Wherein Xaa is hydroxyproline
AKPSYXXTYK

SEQ ID NO: 140 - protein, 10 aa, Artificial Sequence
  SITE 1: Wherein Ala is N-terminally modified with a 3,4-Dihydrocinnamic acid group
  Peptide component
  SITE 6,9: Wherein Xaa is hydroxyproline
AKPSYXTYXK

SEQ ID NO: 141 - protein, 10 aa, Artificial Sequence
  Peptide component
  SITE 5,8: Wherein Xaa is 3,4-dihydroxyphenylalanine
  SITE 6,9: Wherein Xaa is hydroxyproline
AKPSXXTXXK

SEQ ID NO: 142 - protein, 9 aa, Artificial Sequence
  Peptide component
AKPSPTYPK

SEQ ID NO: 143 - protein, 10 aa, Artificial Sequence
  Peptide component
  SITE 3,6,9: Wherein Xaa is hydroxyproline
AKXSYXTYXK

SEQ ID NO: 144 - protein, 10 aa, Artificial Sequence
  Peptide component
  SITE 3,6,9: Wherein Xaa is hydroxyproline
  SITE 5,8: Wherein Xaa is 3,4-dihydroxyphenylalanine
AKXSXXTXXK

SEQ ID NO: 145 - protein, 10 aa, Artificial Sequence
  Peptide component
  SITE 3,6,9: Wherein Xaa is hydroxyproline
  SITE 8: Wherein Xaa is 3,4-dihydroxyphenylalanine
AKXSYXTXXK

SEQ ID NO: 146 - protein, 9 aa, Artificial Sequence
  SITE 1: Wherein Ala is N-terminally modified with a 3,4-Dihydrocinnamic acid group
  Peptide component
  SITE 6,7: Wherein Xaa is hydroxyproline
AKPSYXXTY

SEQ ID NO: 147 - protein, 9 aa, Artificial Sequence
  SITE 1: Wherein Ala is N-terminally modified with a 3,4-Dihydrocinnamic acid group
  Peptide component
  SITE 6,9: Wherein Xaa is hydroxyproline
AKPSYXTYX

SEQ ID NO: 148 - protein, 9 aa, Artificial Sequence
  Peptide component
  SITE 5,8: Wherein Xaa is 3,4-dihydroxyphenylalanine
  SITE 6,9: Wherein Xaa is hydroxyproline
AKPSXXTXX

SEQ ID NO: 149 - protein, 8 aa, Artificial Sequence
  Peptide component
AKPSPTYP

SEQ ID NO: 150 - protein, 9 aa, Artificial Sequence
  Peptide component
  SITE 3,6,9: Wherein Xaa is hydroxyproline
AKXSYXTYX

SEQ ID NO: 151 - protein, 9 aa, Artificial Sequence
  Peptide component
  SITE 3,6,9: Wherein Xaa is hydroxyproline
  SITE 5,8: Wherein Xaa is 3,4-dihydroxyphenylalanine
AKXSXXTXX

SEQ ID NO: 152 - protein, 9 aa, Artificial Sequence
  Peptide component
  SITE 3,6,9: Wherein Xaa is hydroxyproline
  SITE 8: Wherein Xaa is 3,4-dihydroxyphenylalanine
AKXSYXTXX

SEQ ID NO: 153 - protein, 10 aa, Artificial Sequence
  Peptide component
  SITE 5: Wherein Xaa is 3,4-dihydroxyphenylalanine
  SITE 6,10: Wherein Xaa is hydroxyproline
AKPSXXTYKX

SEQ ID NO: 154 - protein, 10 aa, Artificial Sequence
  Peptide component
  SITE 3,6,10: Wherein Xaa is hydroxyproline
  SITE 5: Wherein Xaa is 3,4-dihydroxyphenylalanine
AKXSXXTYKX

SEQ ID NO: 155 - protein, 10 aa, Artificial Sequence
  Peptide component
  SITE 6,10: Wherein Xaa is hydroxyproline
AKPSYXTYKX

SEQ ID NO: 156 - protein, 10 aa, Artificial Sequence
  Peptide component
  SITE 3,6,10: Wherein Xaa is hydroxyproline
AKXSYXTYKX

SEQ ID NO: 157 - protein, 10 aa, Artificial Sequence
  Peptide component
  SITE 6,10: Wherein Xaa is hydroxyproline
  SITE 8: Wherein Xaa is 3,4-dihydroxyphenylalanine
AKPSYXTXKX

SEQ ID NO: 158 - protein, 10 aa, Artificial Sequence
  Peptide component
  SITE 3,6,10: Wherein Xaa is hydroxyproline
  SITE 8: Wherein Xaa is 3,4-dihydroxyphenylalanine
AKXSYXTXKX

SEQ ID NO: 159 - protein, 10 aa, Artificial Sequence
  Wherein Xaa is hydroxyproline
  SITE 5,8: Wherein Xaa is 3,4-dihydroxyphenylalanine
  SITE 6,10: Wherein Xaa is hydroxyproline
AKPSXXTXKX

SEQ ID NO: 160 - protein, 10 aa, Artificial Sequence
  Peptide component
  SITE 3,6,10: Wherein Xaa is hydroxyproline
  SITE 5,8: Wherein Xaa is 3,4-dihydroxyphenylalanine
AKXSXXTXKX

SEQ ID NO: 161 - protein, 7 aa, Artificial Sequence
  Peptide component
  VARIANT 2: Wherein Xaa is proline, hydroxyproline, or dihydroxyproline
  VARIANT 4: Wherein Xaa is tyrosine, 3,4-dihydroxyphenylalanine or is absent
  VARIANT 5: Wherein Xaa is serine, proline, hydroxyproline or dihydroxyproline
  VARIANT 6: Wherein Xaa is proline, hydroxyproline, dihydroxyproline, threonine, 3,4-dihydroxyphenylalanine, or tyrosine
KXSXXXK

SEQ ID NO: 162 - protein, 7 aa, Artificial Sequence
  Peptide component
  SITE 5: Wherein Xaa is hydroxyproline
  SITE 6: Wherein Xaa is 3,4-dihydroxyphenylalanine
KPSYXXK

SEQ ID NO: 163 - protein, 7 aa, Artificial Sequence
  Peptide component
  SITE 5: Wherein Xaa is hydroxyproline
KPSYXYK

SEQ ID NO: 164 - protein, 10 aa, Artificial Sequence
  SITE 1: Wherein Ala is N-terminally modified with a molecule selected from NOTA, TETA, ATSM, PTSM, and CB-TE2A, or is not so modified.
  Peptide component
  SITE 6: Wherein Xaa is hydroxyproline
  SITE 9: Wherein Xaa is 3,4-dihydroxyphenylalanine
AKPSYXTYXK

SEQ ID NO: 165 - protein, 12 aa, Artificial Sequence
  REPEAT 1..10: Wherein the sequence is repeated two times
  REPEAT 1..11: Wherein the sequence is repeated 2 times
  Peptide component
  SITE 5: Wherein Tyr is optionally Iodinated with Iodine 125 or is not so modified.
  SITE 6,9: Wherein Xaa is hydroxyproline
  SITE 8: Wherein Xaa is 3,4-dihydroxyphenylalanine
  SITE 8: Wherein Xaa is optionally Iodinated with Iodine 125 or is not so modified.
AKPSYXTXXKKK

SEQ ID NO: 166 - protein, 13 aa, Artificial Sequence
  SITE 1: Wherein Xaa is aminocaprioic acid
  REPEAT 1..11: Wherein the sequence is repeated 2 times
  REPEAT 1..12: Wherein the sequence is repeated 2 times
  Peptide component
  SITE 7,10: Wherein Xaa is hydroxyproline
  SITE 9: Wherein Xaa is 3,4-dihydroxyphenylalanine
XAKPSYXTXXKKK

SEQ ID NO: 167 - protein, 13 aa, Artificial Sequence
  MOD_RES 1: Wherein Xaa is N-terminally conjugated to a Fluorescein isothiocyanate isomer I moiety
  SITE 1: Wherein Xaa is aminocaprioic acid
  REPEAT 1..11: Wherein the sequence is repeated 2 times
  SITE 1..12: Wherein the sequence is repeated 2 times
  Fluorescin-conjugated peptide component
  SITE 7,10: Wherein Xaa is hydroxyproline
  SITE 9: Wherein Xaa is 3,4-dihydroxyphenylalanine
XAKPSYXTXXKKK

SEQ ID NO: 168 - protein, 4 aa, Artificial Sequence
  Amino acid sequence defined by Y
  VARIANT 2,3: Wherein Xaa is selected from the group proline, alanine, hydroxyproline, threonine, 3,4-dihydroxyphenylalanine, and tyrosine
PXXK

SEQ ID NO: 169 - protein, 4 aa, Artificial Sequence
  SITE 1: Wherein Xaa is hydroxyproline
  Amino acid sequence defined by Y
  VARIANT 2,3: Wherein Xaa is selected from the group proline, alanine, hydroxyproline, threonine, 3,4-dihydroxyphenylalanine, and tyrosine
XXXK

SEQ ID NO: 170 - protein, 4 aa, Artificial Sequence
  Amino acid sequence defined by Y
  VARIANT 2,3: Wherein Xaa is selected from the group proline, alanine, hydroxyproline, threonine, 3,4-dihydroxyphenylalanine, and tyrosine
TXXK

SEQ ID NO: 171 - protein, 4 aa, Artificial Sequence
  Amino acid sequence defined by Y
  SITE 3: Wherein Xaa is 3,4-dihydroxyphenylalanine
PTXK

SEQ ID NO: 172 - protein, 4 aa, Artificial Sequence
  Amino acid sequence defined by Y
PTYK

SEQ ID NO: 173 - protein, 4 aa, Artificial Sequence
  Amino acid sequence defined by Y
TYPK

SEQ ID NO: 174 - protein, 4 aa, Artificial Sequence
  SITE 1: Wherein Xaa is hydroxyproline
  Amino acid sequence defined by Y
XTYK

SEQ ID NO: 175 - protein, 4 aa, Artificial Sequence
  SITE 1: Wherein Xaa is hydroxyproline
  Amino acid sequence defined by Y
  SITE 3: Wherein Xaa is 3,4-dihydroxyphenylalanine
XTXK

SEQ ID NO: 176 - protein, 4 aa, Artificial Sequence
  SITE 1: Wherein Xaa is hydroxyproline
  Amino acid sequence defined by Y
XTAK

SEQ ID NO: 177 - protein, 4 aa, Artificial Sequence
  Amino acid sequence defined by Y
  SITE 3: Wherein Xaa is hydroxyproline
TYXK

SEQ ID NO: 178 - protein, 4 aa, Artificial Sequence
  Amino acid sequence defined by Y
  SITE 2: Wherein Xaa is 3,4-dihydroxyphenylalanine
  SITE 3: Wherein Xaa is hydroxyproline
TXXK

SEQ ID NO: 179 - protein, 4 aa, Artificial Sequence
  Amino acid sequence defined by Y
  SITE 3: Wherein Xaa is hydroxyproline
TAXK

SEQ ID NO: 180 - protein, 5 aa, Artificial Sequence
  SITE 1,4: Wherein Xaa is hydroxyproline
  Amino acid sequence defined by Y
XTYXK

SEQ ID NO: 181 - protein, 5 aa, Artificial Sequence
  Amino acid sequence defined by Y
  SITE 3: Wherein Xaa is hydroxyproline
  SITE 5: Wherein Xaa is 3,4-dihydroxyphenylalanine
TYXKX

SEQ ID NO: 182 - protein, 4 aa, Artificial Sequence
  Amino acid sequence defined by Y
  SITE 3: Wherein Xaa is 3,4-dihydroxyphenylalanine
TYXK